(19) United States
(12) Patent Application Publication
(10) Pub. No.: US 2022/0246862 A1
(43) Pub. Date: Aug. 4, 2022

(54) ORGANIC COMPOUND AND BLUE ORGANIC LIGHT EMITTING DIODE USING THE SAME

(71) Applicants: TSUN-YUAN HUANG, (US); CHING-YAN CHAO, (US); FENG-WEN YEN, (US)

(72) Inventors: TSUN-YUAN HUANG, CHIAYI (TW); CHING-YAN CHAO, HSIN-CHU (TW); FENG-WEN YEN, TAIPEI (TW)

(73) Assignee: LUMINESCENCE TECHNOLOGY CORP., HSIN-CHU (TW)

(21) Appl. No.: 17/453,856

(22) Filed: Nov. 7, 2021

(30) Foreign Application Priority Data
Jan. 29, 2021 (TW) 110103532

(51) Int. Cl. H01L 51/00

(52) U.S. Cl. CPC H01L 51/0073; H01L 51/0067; H01L 51/5096; H01L 51/0072; H01L 51/0074; H01L 51/0052

(57) ABSTRACT

An organic compound having one the following structure of formula (1) is described:

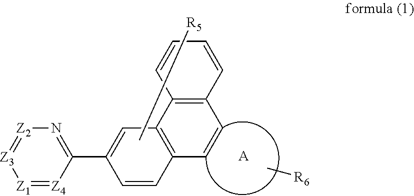

The application in a blue OLED is also described.

### CROSS-REFERENCE TO RELATED APPLICATION

This application claims the benefit of priority of TW Patent Application Ser. No. 110103532 filed on Jan. 29, 2021, the content of which is incorporated herein by reference in its entirety.

### BACKGROUND

### Field of Invention

The present invention relates generally to a compound, and, more specifically, to a blue organic light emitting device using the compound.

### Description of Related Art

Organic electroluminescence (organic EL) devices, i.e., organic light emitting diodes (OLEDs) are devices having planar light sources. As organic light emitting diodes have many advantages, such as self-emission, high brightness, light weight, ultra-thin profile, low power consumption, wide angle of view, high contrast, easy fabrication, and fast response time, so they can be widely used in displays, such as passive OLED (PMOLED display) and active OLED display (AMOLED display). In recent years, OLEDs have also gradually been applied to lighting-related industries, such as products of room lighting or automotive lighting recently attracted more attention.

A typical OLED is made by vacuum evaporation, in which a hole transport layer, a light emitting layer, an electron transport layer and a cathode are sequentially evaporated on a pre-patterned indium tin oxide (ITO) substrate, to form a multi-layer thin films structure. After voltage is applied to the OLED, holes and electrons are injected into the hole transport layer and the electron transport layer from the anode (ITO) and the cathode, respectively, and to form excitons in the light emitting layer. The excitons make the electrons and holes recombine to emit light in relaxation

Generally, an OLED device may have monochromatic light color, such as red, yellow, green or blue by selecting materials for a light-emitting layer. Moreover, by being combined with the design of the device structure, an OLED device may have composite light colors such as white light or purple light. However, among current OLED devices, the efficiency and lifetime of blue OLED devices are significantly lower than that of red or green OLED devices. A light emitting layer of a blue OLED device is operated by mechanism of fluorescent. The light emitting layer operated by mechanism of fluorescent has efficiency less than half of a light emitting layer operated by mechanism of phosphorescence. Therefore, the blue OLED device having the light emitting layer has short lifetime. For the same reason, white OLED devices composed of yellow-blue, red-yellow-blue or red-green-blue may also have short lifetime. In a display or lighting equipment, such a white OLED device will gradually have the problem of warmer red shift, after the white OLED device illuminate for a long time. The lighting equipment and the display equipment such as a PMOLED and AMOLED display are required to have an increased lifetime. The requirement is an urgent issue for the current OLED industry to improve.

As used herein, “top” means furthest away from the substrate, while “bottom” means closest to the substrate. Where a second layer is described as formed on or onto a first layer, the second layer is formed further away from substrate. There may be other layers between the second layer and the first layer, unless it is specified that the second layer is “in contact with” the first layer. For example, a cathode may be described as formed onto an anode, even though there are various organic layers in between.

### SUMMARY

An aspect of the present disclosure provides an organic compound is represented by the following formula (1):

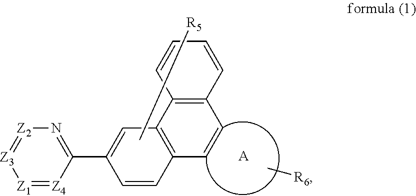

wherein A represents dibenzofuran, dibenzothiophene, dibenzoselenophene, 9,9-dialkylfluorene, 9,9-dialkyl-9H-9-silafluorenyl or carbazole;

where Z₁ is N or CR₁;

where Z₂ is N or CR₂;

where Z₃ is N or CR₃;

where Z₄ is N or CR₄;

wherein at least one of Z₃ and Z₄ is N;

wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;

wherein each of R₅ and R₆ represents mono to a maximum possible number of substitutions, or no substitution; and

each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof.

According to one embodiment of the present disclosure, one of the following is true:

Z₁ is CR₁, and Z₂ is CR₂;

at least one of R₁ and R₂ is selected from the group consisting of 3-biphenyl, 2-biphenyl, 4-biphenyl, 9,9-dialkylfluorenyl, dibenzofuranyl, dibenzothienyl, and combinations thereof if R₁ and R₂ are not the same;

R₅ or R₆ is selected from the group consisting of unsubstituted, methyl, ethyl, isopropyl, n-butyl, n-hexyl, and combinations thereof if R₁ and R₂ are both phenyl;

if one of R₁ and R₂ is 3-biphenyl, the other is phenyl;

Z₂ is CR₂, and Z₃ is CR₃;

R₂ and R₃ are connected so that Z₂ and Z₃ together form naphthalene or benzene;

Z₃ is N; and

Each of R₅ and R₆ is independently selected from the group consisting of methyl, ethyl, isopropyl, n-butyl, n-hexyl, and combinations thereof.

Another aspect of the present disclosure, an organic compound is provided for extending lifetime or increasing efficiency of a blue organic light emitting device. The organic compound being represented by one of the following formulas (2) to (6):

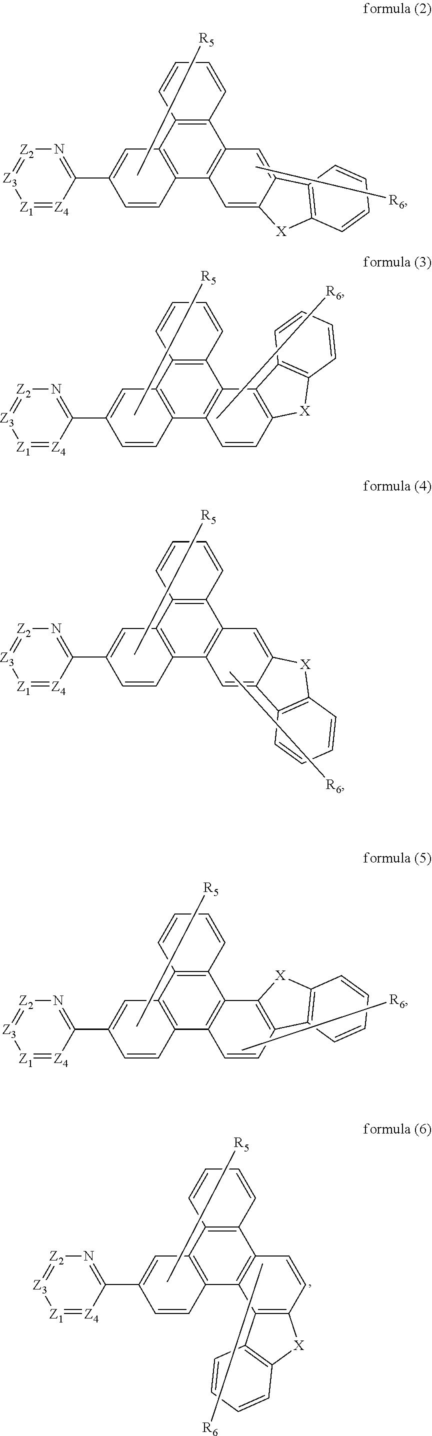

where Z₁ is N or CR₁;

where Z₂ is N or CR₂;

where Z₃ is N or CR₃;

where Z₄ is N or CR₄;

wherein at least one of Z₃ and Z₄ is N;

wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;

wherein each of R₅ and R₆ represents mono to a maximum possible number of substitutions, or no substitution;

each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and

one of the following is true:
R₁ and R₂ are not the same;
Z₁ is CR₁, and Z₂ is CR₂;
R₅ or R₆ is a mono substituent if R₁ and R₂ are both phenyl,
at least one of R₁ and R₂ is selected from the group consisting of 3-biphenyl, 2-biphenyl, 4-biphenyl, 9,9-dialkylfluorenyl, phenyl, methylphenyl, methyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, or 2-(4-pyridyl)phenyl, dibenzofuranyl, dibenzothienyl, dibenzoselenophenyl, and combinations thereof;
if one of R₁ and R₂ is 3-biphenyl, 2-biphenyl, or 4-biphenyl, the other is phenyl;
R₂ and R₃ are connected so that Z₂ and Z₃ together form a polycyclic aromatic group;
R₁ to R₄ are independently selected from the group consisting of H, phenyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3- (4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and
each of R₅ and R₆ is independently selected from the group consisting of no substitution, methyl, ethyl, propyl, butyl, hexyl, and combinations thereof.

According to one embodiment of the present disclosure, one of the following is true:

Z₁ is CR₁, and Z₂ is CR₂;

at least one of R₁ and R₂ is selected from the group consisting of 3-biphenyl, 2-biphenyl, 4-biphenyl, 9,9-dialkylfluorenyl, dibenzofuranyl, dibenzothienyl, and combinations thereof if R₁ and R₂ are not the same;

R₅ or R₆ is selected from the group consisting of unsubstituted, methyl, ethyl, isopropyl, n-butyl, n-hexyl, and combinations thereof if R₁ and R₂ are both phenyl;

if one of R₁ and R₂ is 3-biphenyl, the other is phenyl;

Z₂ is CR₂, and Z₃ is CR₃;

R₂ and R₃ are connected so that Z₂ and Z₃ together form naphthalene or benzene;

Z₃ is N; and

Each of R₅ and R₆ is independently selected from the group consisting of methyl, ethyl, isopropyl, n-butyl, n-hexyl, and combinations thereof.

According to one or more aspects of the present disclosure, a blue organic light emitting diode comprises a first electrode, a second electrode, and an organic thin film layer between the first electrode and the second electrode, wherein the organic thin film layer is selected from the group consisting of a light emitting layer, a hole blocking layer, an electron transport layer, and combinations thereof, wherein the first light emitting layer comprises a first host and a first guest, and wherein the organic thin film layer comprises the organic compound of the present disclosure.

According to one or more aspects of the present disclosure, the first host comprises the organic compound as a first material. The first hole blocking layer comprises the organic compound as a second material. The second material is the same as the first material.

According to one or more aspects of the present disclosure, the blue organic light emitting diode further comprises a hole blocking layer between the light emitting layer and the electron transport layer.

According to one or more aspects of the present disclosure, the hole blocking layer or the electron transport layer comprises one of the following compounds:

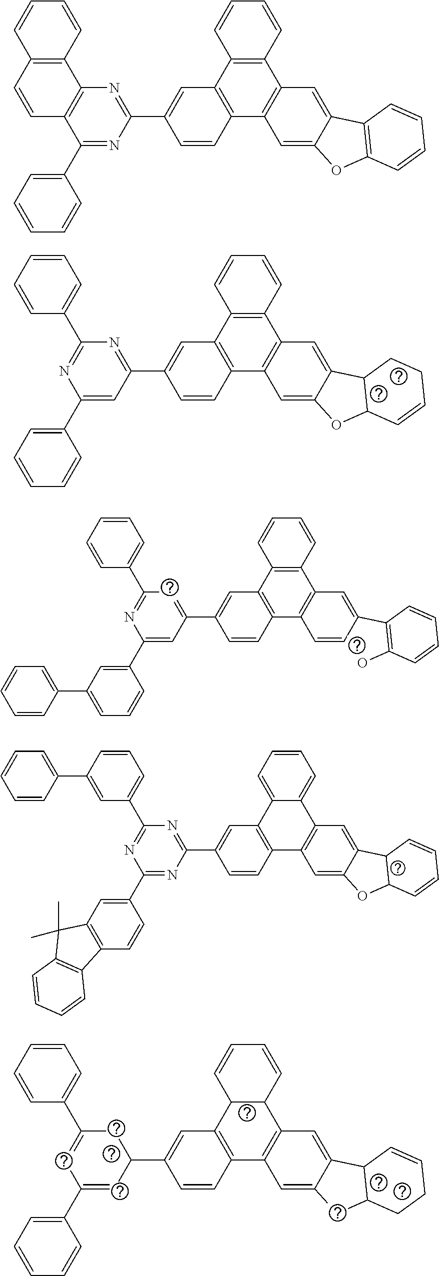

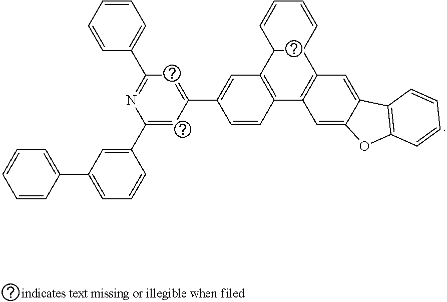 [SMILES: C1=CC2c3cc(-c4cc(-c5cccc(-c6ccccc6)c5)nc(-c5ccccc5)n4)ccc3-c3cc4oc5ccccc5c4cc3C2C=C1]

Preferably, the hole blocking layer or the electron transport layer comprises the same compound.

### BRIEF DESCRIPTION OF THE DRAWINGS

FIG. 1 is a schematic view showing a first embodiment of the present disclosure.

FIG. 2 is a schematic view showing a second embodiment of the present disclosure.

### DETAILED DESCRIPTION

Plural embodiments of the present disclosure are disclosed through drawings. For the purpose of clear illustration, many practical details will be illustrated along with the description below. It should be understood that, however, these practical details should not limit the present disclosure. In other words, in embodiments of the present disclosure, these practical details are not necessary. In addition, for the purpose of simplifying the drawings, some conventional structures and components are simply and schematically depicted in the figures.

Aiming at a gap or a specific layer calibrated by a receptor component, to research and develop new organic compounds having functions of OLED photoelectric mechanism and actually solving technical problems, is not only one of the main ideas and approaches in the research and development, but also key technical basis for filing patent applications. The above-mentioned research methods of photoelectric mechanism have become the key work for the research and development of organic compounds in this field. Therefore, it is not suitable to deviate from the research methods in this field when determining the technical problems actually solved by the present disclosure. When the concept of the present disclosure is analyzed, it will help to more accurately determine the technical problems actually solved by the compound invention, and to lay a solid foundation for the judgment of technical enlightenment, if the above-mentioned research methods of those skilled in the art is followed, if the process of inventing an organic compound and a blue OLED using the same is experienced, and if the applicant's technical contributions are essentially grasped.

It is to be understood that although particular phrases used herein, such as “first”, “second”, “third”, and so on, are used to describe different components, members, regions, layers, and/or sections, these components, members, regions, layers, and/or sections should not be limited by these terms. These phrases are used to distinguish one component, member, region, layer, or section from another component, member, region, layer, or section. In this way, a first component, member, region, layer, and/or section to be described below may be referred to as a second component, member, region, layer, and/or section, without departing from the spirit and scope of the present disclosure.

Spatially relative phrases, such as “onto”, “on”, “over”, “under”, “below”, “underlying”, “beneath”, “above”, and so on used herein, are used for facilitating description of a relation between one component or feature and another component or feature depicted in the drawings. Therefore, it can be understood that, in addition to directions depicted in the drawings, the spatially relative terms mean to include all different orientations during usage or operations of the device. For example, it is assumed that a device in a figure is reversed upside down, a component described as being “under”, “below”, or “beneath” another component or feature is oriented “onto” or “on” the other component or feature. Therefore, these exemplary terms “under” and “below” may include orientations above and below. The device may be otherwise oriented (e.g., turned by 90 degrees, or other orientations), and the spatially relative terms used herein should be explained accordingly.

Accordingly, it may be understood that when a component or a layer is referred to as being “onto”, “on”, “connected to”, or “coupled to” another component or another layer, it may be immediately on the other component or layer, or connected to or coupled to the other component or layer, or there may be one or more intermediate components or intermediate layers. Further, it can be understood that when a component or a layer is referred to as being “between” two components or two layers, it may be the only component or layer between the two components or layers, or there may be one or more intermediate components or intermediate layers.

Terminologies used herein are only for the purpose of describing particular embodiments, but not limiting the present disclosure. The singular form of “a” and “the” used herein may also include the plural form, unless otherwise indicated in the context. Accordingly, it can be understood that when there terms “include” or “comprise” are used in the specification, it clearly illustrates the existence of a specified feature, bulk, step, operation, component, and/or member, while not excluding the existence or addition of one or more features, bulks, steps, operations, components, members and/or groups thereof. “And/or” used herein includes any and all combinations of one or more related terms that are listed. When a leading word, such as “at least one of”, is added ahead of a component list, it is to describe the entire component list, but not individual components among the list.

It is to be understood that when a molecular fragment is described as being a substituent or otherwise attached to another moiety, its name may be written as if it were a fragment (e.g., dibenzofuranyl, naphthyl, biphenyl, arylene, phenyl, phenylene) or as if it were the whole molecule (e.g., dibenzofuran, naphthalene, biphenyl, aromatic group, benzene). As used herein, these different ways of designating a substituent or attached fragment are considered to be equivalent.

The terms “substituted” and “substitution” refer to a substituent bonded to the relevant position, e.g., a carbon or nitrogen. Accordingly, for example, when R₁ represents mono substitution, one R₁ must not be H. Similarly, when R₁ represents at least di substitutions, at least two R₁ must not be H. In addition, when R₁ represents no substitution, R₁, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine.

As used herein, a maximum possible number of substitutions in a ring structure will depend on the total number of available valencies in the ring atoms. See

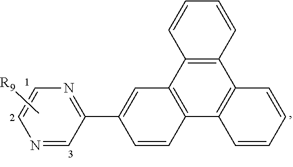

for example, there are three available valencies in the ring atoms of the left heteroaryl ring, so the maximum possible number of substitutions of R₉ substitution is 3.

As used herein, when a single ring of a compound's formula is substituted a non-symmetrical polycyclic aryl, the formula may represent at least two compounds. For example, the formula

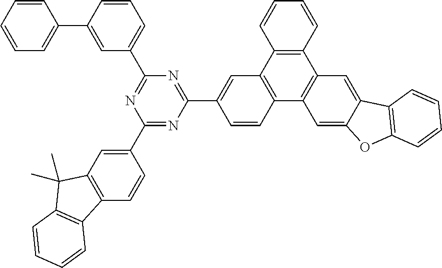 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

represents a first compound

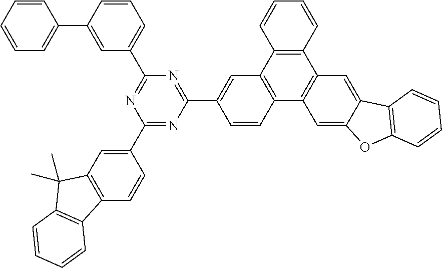 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

and represents a second compound

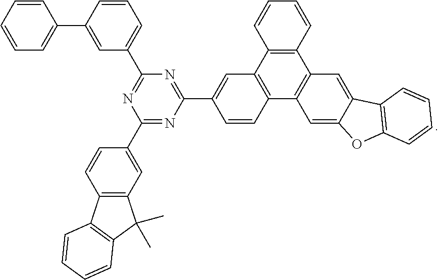 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

Even if the scope of the patent application only draws the first compound, it is not limited to it.

That is, the formula of the first compound is to further comprises the second compound. See the following for reference.

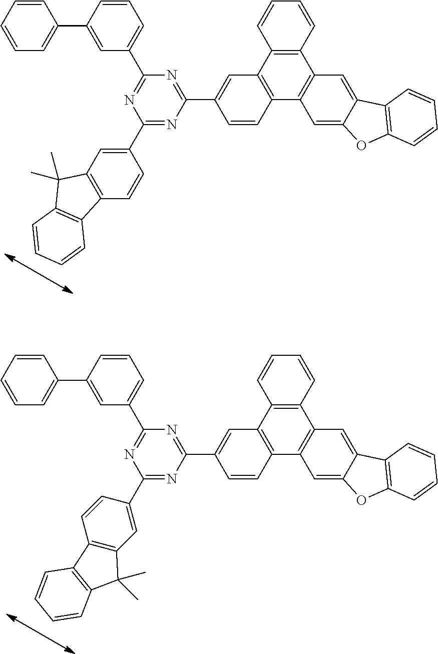 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21]

As used herein, the terms “R₁”, “R₂”, “R₃”, “R₄”, “R₅”, “R₆” . . . or “R₇” may independently be hydrogen or a substituent selected from the group consisting of aryl, alkyl, alkylphenyl, halogen, phenyl, methylphenyl, pyridyl, biphenyl, pyridylphenyl, 9,9-dialkylfluorenyl, dibenzofuranyl, two Benzothienyl, carbazolyl, methyl, butyl, n-butyl, hexyl, propyl, isopropyl, hexylphenyl, triazinyl, diazinyl, naphthyl, heteroaryl, aralkyl , Trifluoromethyl, cyano, nitro, trimethylsilyl, silyl, aryl substituted or unsubstituted by methyl or hexyl, biphenyl, pyridylphenyl, m-terphenyl, diiso Butylcarbazolyl, phenylcarbazolyl, dimethylcarbazolyl, cyano, phenyl, dicyanophenyl, nitro, cycloalkyl, heterocycloalkyl, aromatic group, aromatic amine Group, heteroarylamine group, deuterium, alkoxy, amino, silyl, fluorenyl, benzofluorenyl, anthracenyl, phenanthryl, pyrenyl, alkenyl, cycloalkenyl, heteroalkenyl, alkynyl, carbonyl , Carboxylic acid, ether, ester, glycol, isonitrile, thio, sulfinamide, sulfonyl, phosphoric acid, triphenylene, benzimidazole, dicarbazolyl, diphenylphosphine oxide, Phenanthroline group, dihydroacridinyl, phenothiazinyl, phenoxazinyl, dihydrophenazinyl, diphenylamino, triphenylamino, phenyldibenzofuranylamino, phenyldiphenyl Thioanilino, and combinations thereof. In addition, when each of R, R₁, R₂, R₃, R₄, R₅, R₆ . . . or R_{N} represents a substituent, two adjacent substituents, for example, two adjacent R substituents may be optionally bonded (connected) or fused together to form a single ring structure (such as benzene), or to form fused rings (also polycyclic aromatic groups such as naphthalene. The fused rings are formed together with the substituted.

As used herein, if the term “a first integer to a second integer” is used to express a plurality of solutions, it may cover the first integer, the second integer, and each integer between the first and the second integers. That is to say, when the term “a first integer to a second integer” expresses a plurality of solutions, all of its integers are parallel technical solutions. In this case, the term “a first integer to a second integer” is not used to express a numerical range. For example, 1 to 4 covers 1, 2, 3, 4 and does not include 1.5. For another example, 0 to 3 cover 0, 1, 2, and 3, wherein 0, 1, 2, and 3 are technical solutions in parallel. For another example, 1 to 5 covers 1, 2, 3, 4, and 5, among which 1, 2, 3, 4, and 5 are parallel technical solutions. These solutions may be, for example, the number of substituents or the number of carbon atoms. It is noted that “a maximum possible number of substitutions” is also a kind of integer.

As used herein, “combinations thereof” indicates that one or more members of the applicable list are combined to form a known or chemically stable arrangement that one of ordinary skill in the art can envision from the applicable list. For example, two phenyl groups can be combined (bonded) to form a biphenyl group. If a methyl phenyl group substituted for a first C is combined to a methyl group substituted for a second C adjacent to the first C, a naphthalene is formed (together with the adjacent Cs). The monocyclic aromatic group and the polycyclic aromatic group can be bonded (connected) together through a direct bond (single bond), or can be condensed to form two adjoining rings in which two carbons are common the two adjoining rings. Alkyl and deuterium can be combined to form partially or fully deuterated alkyl. Halogen and alkyl can be combined to form haloalkyl. Halogen, alkyl and aryl can be combined to form haloaralkyl.

The following description of various terms related to substituents, one of the intentions, is to show that some substituents are mutually replaceable and/or have common functions. In other words, in the field of the present disclosure, these substituents may be of the same category.

The term “alkyl” refers to and includes both straight and branched chain alkyl radicals. Preferred alkyl groups are those containing 30 or fewer carbon atoms, preferably 1 to 20 carbon atoms, more preferably 1 to 15 carbon atoms, and most preferably 1 to 12 carbon atoms. Suitable alkyl groups include methyl, ethyl, propyl, isopropyl, butyl, n-butyl, hexyl, n-hexyl, 1-methylethyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group is optionally substituted.

The term “aryl” or “aromatic group” as used herein are interchangeable with each other and contemplates monocyclic aromatic groups (or hydrocarbyls), polycyclic aromatic groups (or hydrocarbyls), fused ring hydrocarbon units, and combinations thereof. The polycyclic aromatic group may have two, three, four, five, or more rings in which two carbons are common to two adjoining rings (meaning that the two adjacent rings are “fused”). A polycyclic aromatic group can be named a bicyclic aromatic group if it has two rings; if it has three rings, it can be named a tricyclic aromatic group, and so on. In a polycyclic aromatic group, at least one of the polycyclic rings is an aromatic group, e.g., the other rings can be cycloalkyls, cycloalkenyls, aryl, heterocycles, and/or heteroaryls. In terms of the number of carbon atoms, preferred aromatic groups are those containing 30 or fewer carbon atoms, preferably 6 to 30 carbon atoms, more preferably 6 to 20 carbon atoms, and most preferably 6, 10 or 12 carbon atoms.

Suitable aromatic groups include but not limited to phenyl, 3-biphenyl, 2-biphenyl, 4-biphenyl, fluorenyl, fluoranthene, benzanthracene, benzo[c]phenanthrene, benzofluorene, 9,9-dialkylfluorenyl, 9,9-dimethylfluorene, naphthalene, phenanthrene, anthracene, triphenylene, pyrene, phenanthrene, perylene, terphenyl, terphenyl, m-terphenyl, p-terphenyl, o-terphenyl, tetraphenyl, phenalene, 9H-fluorene, and azulene.

Among the suitable aromatic groups, preferred include phenyl, 3-biphenyl, 2-biphenyl, 4-biphenyl, fluorenyl, fluoranthene, benzanthracene, benzo[c]phenanthrene, benzofluorene, 9,9-dimethylfluorene Base, naphthalene, phenanthrene, anthracene, triphenylene, pyrene, chrysene, perylene, terphenyl, fluorene and naphthalene. In addition, the above-mentioned “aryl” or “aromatic group” may be optionally substituted, for example, the two hydrogen atoms commonly bonded on the same carbon atom of benzofluorene may be further substituted with two methyl groups, which product may be named dimethyl-benzofluorene. As another example, the phenyl group may be further substituted with methyl, hexyl or pyridyl.

It is noted that a monocyclic azaphenyl group (pyridyl), or a monocyclic azaphenyl group (pyrimidinyl or triazinyl) containing two or more nitrogen atoms that are not adjacent to each other on the ring:

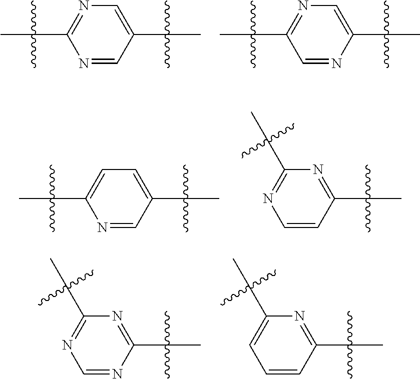 [SMILES: *c1cnc(*)nc1 ; *c1cnc(*)cn1 ; *c1ccc(*)nc1 ; *c1ccnc(*)n1 ; *c1ncnc(*)n1 ; *c1cccc(*)n1]

Like phenylene, they have three pairs of it electrons that resonate with each other and have similar chemical properties. Therefore, if the compound preferably has one of the substituents, other substituents may also be preferred.

As used herein, the terms “heteroaryl” or “heteroaromatic group” are interchangeable with each other. “Heteroaryl” or “heteroaromatic group” may be selected from the group consisting of a monocyclic heteroaromatic group containing one, two, three, four, five or more heteroatoms, a polycyclic heteroaromatic group having two or more rings, and combinations thereof. Heteroatoms include, but are not limited to, O, S, N, P, B, Si and Se. In many cases, O, S, N, Si and Se is the preferred heteroatom. The “monocyclic heteroaromatic group” is preferably a single ring having 5 or 6 ring atoms, and the ring may have one to six heteroatoms. A “polycyclic heteroaromatic group” may have two, three, four, five, six or more rings, where two carbons are common to two adjoining rings (meaning that the rings are “fused”). A polycyclic heteroaromatic group can be named a bicyclic heteroaromatic group if it has two rings; if it has three rings, it can be named a tricyclic heteroaromatic group, and so on. In a polycyclic heteroaromatic group, at least one of the rings is a heteroaryl, e.g., the other rings can be cycloalkyls, cycloalkenyls, aryl, heterocycles, and/or heteroaryls. In terms of the number of carbon atoms, the polycyclic heteroaromatic groups can have from one to six heteroatoms per ring of the polycyclic heteroaromatic groups. Preferred heteroaryl groups are those containing 30 or fewer carbon atoms, preferably 3 to 30 carbon atoms, more preferably 3 to 20 carbon atoms, and most preferably 3 to 12 carbon atoms. Suitable heteroaromatic groups may include dibenzothienyl, dibenzofuranyl, carbazolyl, pyridyl, triazinyl, diazinyl, 1,3,5-triazinyl, quinazoline, quinoxaline, benzoquinazoline , Pyrimidine, dibenzoselenophene, furan, thiophene, benzofuran, benzothiophene, benzoselenophene, indolocarbazole, pyridylindole, pyrrolodipyridine, pyrazole, imidazole, triazole, Oxazole, thiazole, oxadiazole, oxtriazole, dioxazole, thiadiazole, pyridazine, pyrazine, oxazine, oxthiazine, oxadiazine, indole, benzimidazole, indazole, inoxa Oxazine, benzoxazole, benzisoxazole, benzothiazole, quinoline, isoquinoline, cinnoline, naphthyridine, phthalazine, pteridine, xanthene, acridine, phenazine, phenazine thiazine, phenoxazine, benzofuropyridine, furobipyridine, benzothienopyridine, thienodipyridine, benzoselenophenopyridine and selenophenopyridine, 1,2-azaborane, 1,3-azaborane, 1,4-azaborane, borazine, and other nitrogen analogs of the aza-derivatives described above. Among the suitable heteroaromatic groups, preferred include dibenzothiophene, dibenzofuran, carbazole, pyridine, quinazoline, quinoxaline, benzoquinazoline, dibenzoselenophene, indolocarbazole, imidazole, triazine, benzimidazole, 1,2-azaborane, 1,3-azaborane, 1,4-azaborane, borazine, and other nitrogen analogs of the aza-derivatives described above. In addition, the “heteroaryl” or “heteroaromatic group” may be optionally substituted. For example, the carbazolyl group can be further substituted with two isobutyl groups, which is called diisobutylcarbazole.

It is noted that if the compounds drawn in this article, whether in the specification or the scope of the patent application, contain dibenzofuran, the substantial meaning of the drawn dibenzofuran is sufficient to comprise polycyclic heteroaromatic groups containing dibenzothiophene. The O of dibenzofuran and S of dibenzothiophene are elements of the same group. The structure and chemical properties of O and S are very similar, and they may therefore be used instead of each other. For example, even if the scope of the patent application only draws the polycyclic heteroaromatic compound containing dibenzofuran on the left side of the figure, it is not limited to this. In addition, the S of the dibenzothienyl group has to be changed to Se for similar reasons, because Se and S are elements of the same group, with very similar structures and chemical properties, and can be used instead of each other.

If the C of the dialkyl group of the 9,9-dialkylfluorenyl group is changed to Si, the changed group is not for defining a polycyclic aromatic group, but a polycyclic heteroaromatic group. Broadly speaking, Si and C are elements of the same family, their structure and chemical properties are very similar, and they can therefore be used instead of each other. Accordingly, changing the C of the dialkyl group of the 9,9-dialkylfluorenyl group to Si is a suitable example. In addition, the alkyl group attached to Si can also be changed to Rs, and each Rs can be the same or different alkyl groups or other substituents. That is, each Rs can be H (hydrogen) or a substituent selected from the group consisting of deuterium, halogen, alkyl, cycloalkyl, heteroalkyl, heterocycloalkyl, aralkyl, alkoxy, aryloxy, amino, silyl, alkenyl, cycloalkenyl, heteroalkenyl, alkynyl, aryl, heteroaryl, and combinations thereof. Preferred Rs is selected from the group consisting of alkyl, aryl, tolyl, pyridyl, hexylphenyl, naphthyl, and combinations thereof.

Among the listed heteroaromatic groups, the “aza” designation in the fragments i.e., aza-dibenzofuran, aza-dibenzothiophene, etc. means that one or more of the C—H groups in the respective fragment can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

Among the aryl and heteroaryl groups listed above, aryl, alkyl, alkylphenyl, halogen, phenyl, methylphenyl, pyridyl, biphenyl, pyridylphenyl, 9,9-dialkylfluorenyl, dibenzofuranyl, dibenzothienyl, dibenzoselenophene, carbazolyl, methyl, ethyl, isopropyl, n-butyl, n-hexyl, triazine , Pyrimidine, quinoxaline, quinazoline, benzoquinazoline, pyrene, perylene, naphthalene, anthracene, triphenylene, fluoranthene, dimethyl-benzofluorene, phenanthrene, phenanthrene, benzo[c]phenanthrene, benzanthracene, indolocarbazole, imidazole, pyrazine, and benzimidazole, as well as the aza-derivatives or analogs thereof, are of particular interest.

The terms alkyl, heteroaryl, aryl, and heterocyclic group, as used herein, are independently unsubstituted, or independently substituted, with one or more general substituents selected from the group consisting of methyl, ethyl, propyl, butyl, hexyl, halogen, alkyl, phenyl, methylphenyl, pyridyl, biphenyl, pyridylphenyl, 9,9-dialkylfluorenyl, two benzofuranyl, dibenzothienyl, dibenzoselenophene, amino, silyl, cyano, trifluoromethyl, cyanobenzene, dicyanobenzene, benzofluorene, deuterium, halogen, alkyl , cycloalkyl, heteroalkyl, aryl, aralkyl, alkoxy, aryloxy, cycloamino, alkenyl, cycloalkenyl, heteroalkenyl, alkynyl, heteroaryl, acyl, carbonyl , Carboxylic acid, ether, ester, nitrile, isonitrile, thio group, sulfinyl group, sulfinyl group, phosphine group, and combinations thereof.

The terms “halogen” and “halide” are used interchangeably and refer to fluorine, chlorine, bromine, or iodine. The term “trifluoromethyl” refers to a —CF₃ radical. The term “cyano” refers to a —C═N radical. The term “nitro” refers to a -NO₂ radical.

As used herein, abbreviations refer to materials and/or films as follows:

LiQ: 8-hydroxyquinolato-lithium
BH: blue host
BD: blue dopant
EIL: electron injecting layer
ETL: electron transport layer
ETM: electron transport material
EML: emissive layer
EBL: electron blocking layer
HTL: hole transporting layer
HIL: hole injection layer
ITO: indium tin oxide
EL: electroluminescence
HBL: hole blocking layer
Host: host material

As used herein, some of the material structures used in Comparative Example 1 and Comparative Example 2 are as follows:

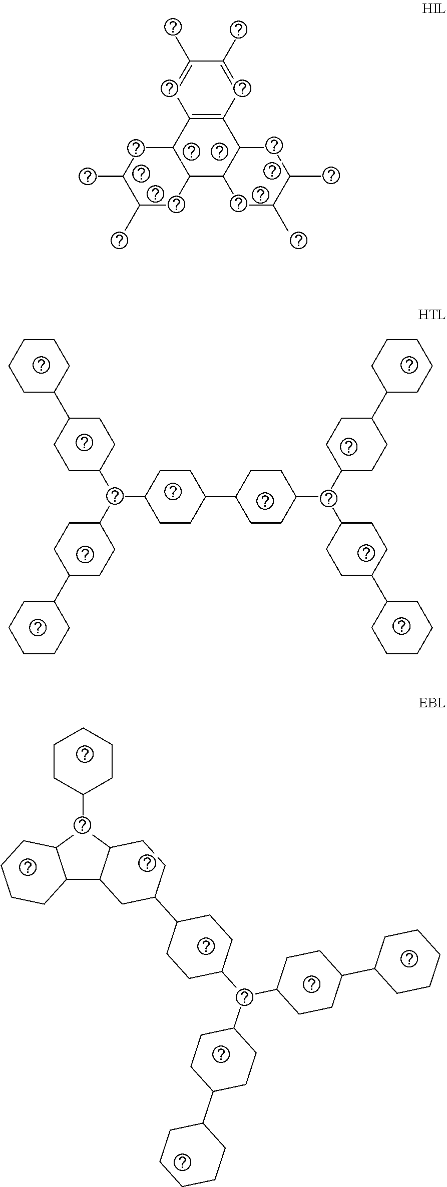

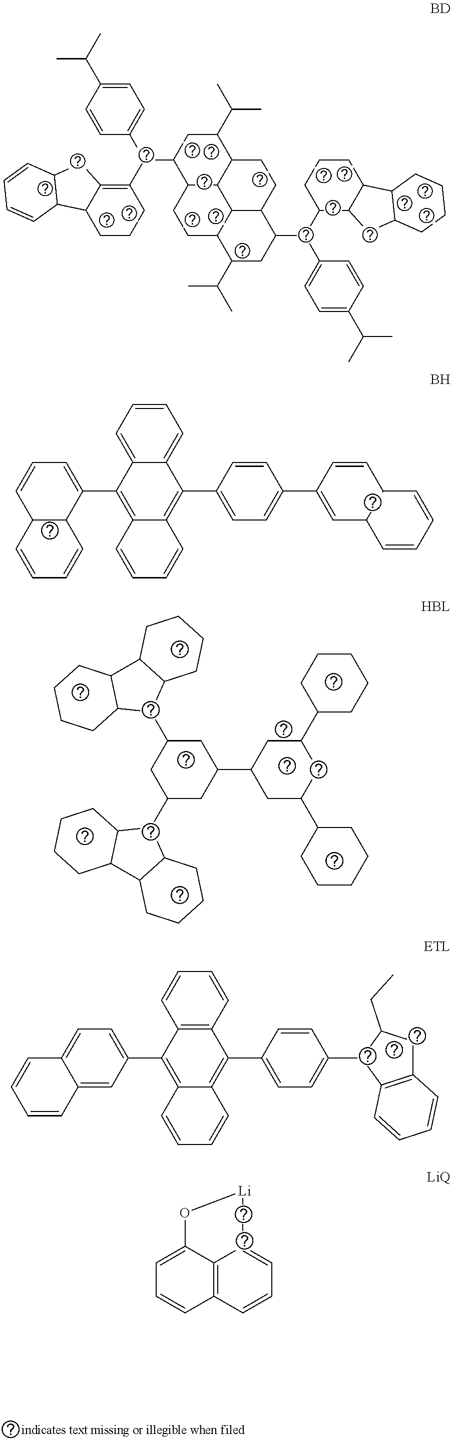

In the present disclosure, after producing a blue OLED, EL spectra and CIE coordination are measured by using a PR650 spectra scan spectrometer. Furthermore, the current/voltage, luminescence/voltage, and yield/voltage characteristics are taken with a Keithley 2400 programmable voltage-current source. The above-mentioned apparatuses are operated at room temperature (about 25° C.) and under atmospheric pressure.

In some embodiments, an organic compound is represented by one of the following formula (1):

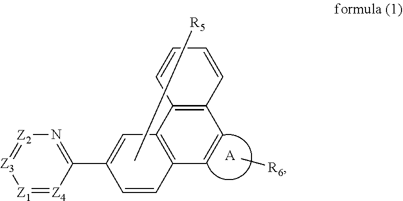

wherein A represents dibenzofuran, dibenzothiophene, dibenzoselenophene, 9,9-dialkylfluorene, 9,9-dialkyl-9H-9-silafluorenyl or carbazole;

where Z₁ is N or CR₁;

where Z₂ is N or CR₂;

where Z₃ is N or CR₃;

where Z₄ is N or CR₄;

wherein at least one of Z₃ and Z₄ is N;

wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;

wherein each of R₅ and R₆ represents mono to a maximum possible number of substitutions, or no substitution; and

each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof.

According to one or more aspects of the present disclosure, one of the following is true:

R₁ and R₂ are not the same;

Z₁ is CR₁, and Z₂ is CR₂;

R₅ or R₆ is a mono substituent if R₁ and R₂ are both phenyl,

at least one of R₁ and R₂ is selected from the group consisting of 3-biphenyl, 2-biphenyl, 4-biphenyl, 9,9-dialkylfluorenyl, phenyl, methylphenyl, methyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, or 2-(4-pyridyl)phenyl, dibenzofuranyl, dibenzothienyl, dibenzoselenophenyl, and combinations thereof;

if one of R₁ and R₂ is 3-biphenyl, 2-biphenyl, or 4-biphenyl, the other is phenyl;

R₂ and R₃ are connected so that Z₂ and Z₃ together form a polycyclic aromatic group;

R₁ to R₄ are independently selected from the group consisting of H, phenyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, dibenzofuranyl, dibenzothienyl, dibenzoselenophenyl, and combinations thereof; and

each of R₅ and R₆ is independently selected from the group consisting of no substitution, methyl, ethyl, propyl, butyl, hexyl, and combinations thereof.

According to one or more aspects of the present disclosure, one of the following is true:

Z₁ is CR₁, and Z₂ is CR₂;

at least one of R₁ and R₂ is selected from the group consisting of 3-biphenyl, 2-biphenyl, 4-biphenyl, 9,9-dialkylfluorenyl, dibenzofuranyl, dibenzothienyl, and combinations thereof if R₁ and R₂ are not the same;

R₅ or R₆ is selected from the group consisting of unsubstituted, methyl, ethyl, isopropyl, n-butyl, n-hexyl, and combinations thereof if R₁ and R₂ are both phenyl;

if one of R₁ and R₂ is 3-biphenyl, the other is phenyl;

Z₂ is CR₂, and Z₃ is CR₃;

R₂ and R₃ are connected so that Z₂ and Z₃ together form naphthalene or benzene;

Z₃ is N; and

Each of R₅ and R₆ is independently selected from the group consisting of methyl, ethyl, isopropyl, n-butyl, n-hexyl, and combinations thereof.

Another aspect of the present disclosure, an organic compound is provided for extending lifetime or increasing efficiency of a blue organic light emitting device. The organic compound being represented by one of the following formulas (2) to (6):

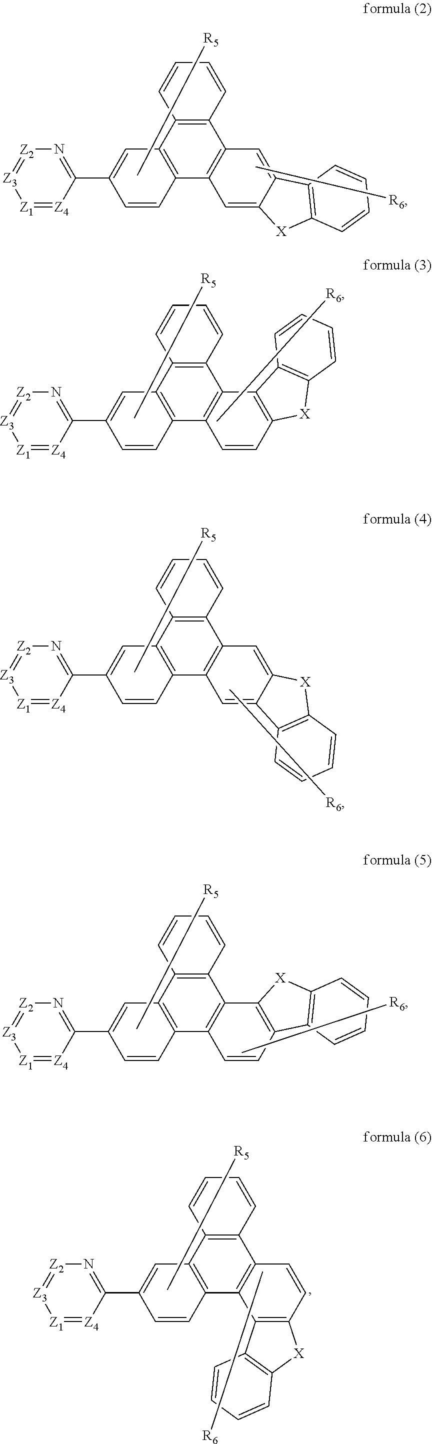

where Z₁ is N or CR₁;

where Z₂ is N or CR₂;

where Z₃ is N or CR₃;

where Z₄ is N or CR₄;

wherein at least one of Z₃ and Z₄ is N;

wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)benzene Group, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;

wherein each of R₅ and R₆ represents mono to a maximum possible number of substitutions, or no substitution;

each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl))Phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and

one of the following is true:
R₁ and R₂ are not the same;
Z₁ is CR₁, and Z₂ is CR₂;
R₅ or R₆ is a mono substituent if R₁ and R₂ are both phenyl,
at least one of R₁ and R₂ is selected from the group consisting of 3-biphenyl, 2-biphenyl, 4-biphenyl, 9,9-dialkylfluorenyl, phenyl, methylphenyl, methyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, or 2-(4-pyridyl)phenyl, dibenzofuranyl, dibenzothienyl, dibenzoselenophenyl, and combinations thereof;
if one of R₁ and R₂ is 3-biphenyl, 2-biphenyl, or 4-biphenyl, the other is phenyl;
R₂ and R₃ are connected so that Z₂ and Z₃ together form a polycyclic aromatic group;
R₁ to R₄ are independently selected from the group consisting of H, phenyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3- (4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and
each of R₅ and R₆ is independently selected from the group consisting of no substitution, methyl, ethyl, propyl, butyl, hexyl, and combinations thereof.

In some embodiments, an organic compound is selected from the group consisting of the following compounds C1 to C184:

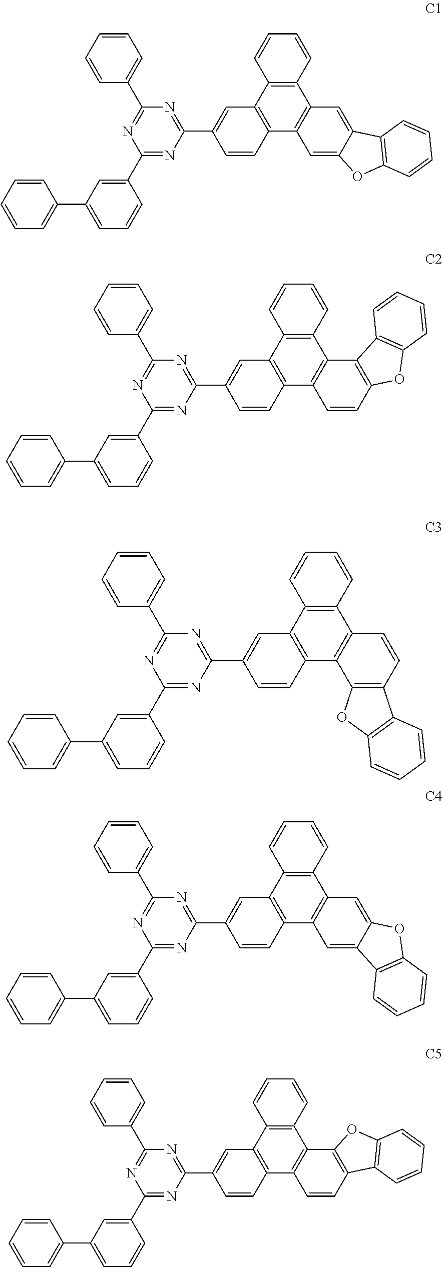 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4ccc6c7ccccc7oc6c45)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1]

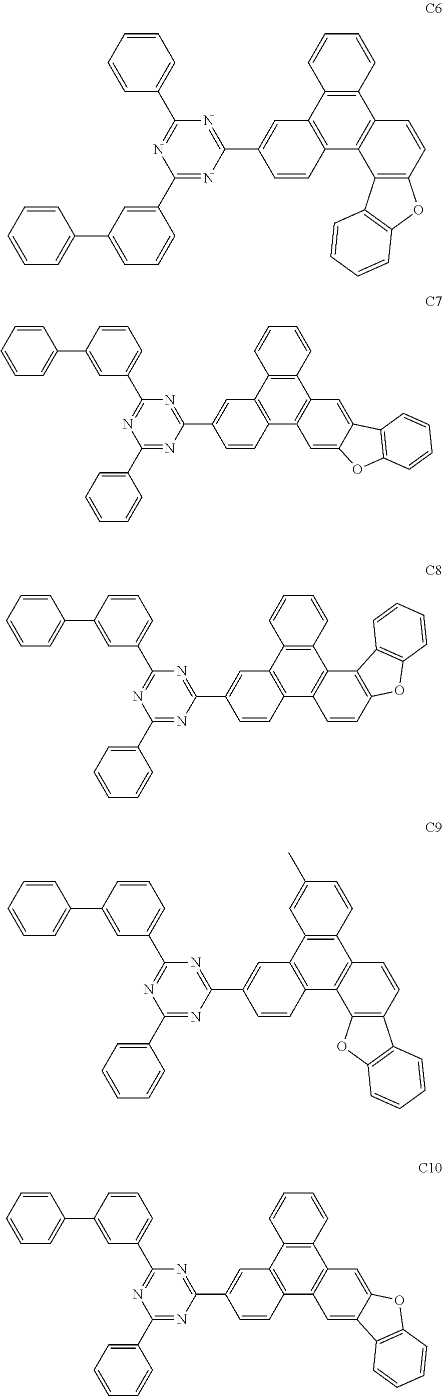 [SMILES: c1ccc(-c2cccc(-c3nc(-c4ccccc4)nc(-c4ccc5c(c4)c4ccccc4c4ccc6oc7ccccc7c6c45)n3)c2)cc1 ; c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; Cc1ccc2c(c1)c1cc(-c3nc(-c4ccccc4)nc(-c4cccc(-c5ccccc5)c4)n3)ccc1c1c2ccc2c3ccccc3oc21 ; c1ccc(-c2cccc(-c3nc(-c4ccccc4)nc(-c4ccc5c(c4)c4ccccc4c4cc6oc7ccccc7c6cc54)n3)c2)cc1]

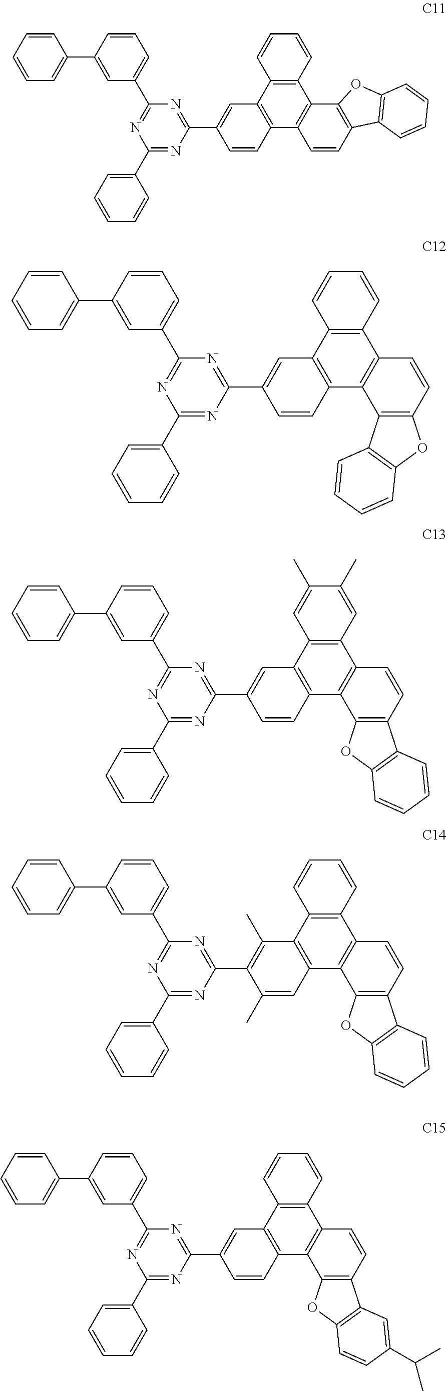 [SMILES: c1ccc(-c2cccc(-c3nc(-c4ccccc4)nc(-c4ccc5c(c4)c4ccccc4c4c5ccc5c6ccccc6oc54)n3)c2)cc1 ; c1ccc(-c2cccc(-c3nc(-c4ccccc4)nc(-c4ccc5c(c4)c4ccccc4c4ccc6oc7ccccc7c6c45)n3)c2)cc1 ; Cc1cc2c3cc(-c4nc(-c5ccccc5)nc(-c5cccc(-c6ccccc6)c5)n4)ccc3c3c(ccc4c5ccccc5oc43)c2cc1C ; Cc1cc2c(c(C)c1-c1nc(-c3ccccc3)nc(-c3cccc(-c4ccccc4)c3)n1)c1ccccc1c1ccc3c4ccccc4oc3c12 ; CC(C)c1ccc2oc3c(ccc4c5ccccc5c5cc(-c6nc(-c7ccccc7)nc(-c7cccc(-c8ccccc8)c7)n6)ccc5c43)c2c1]

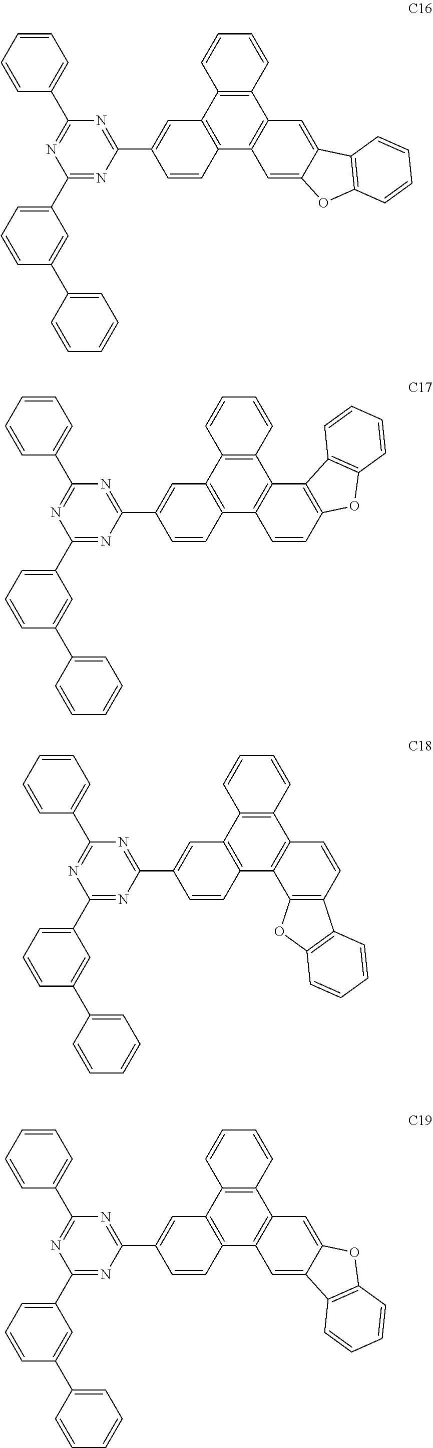

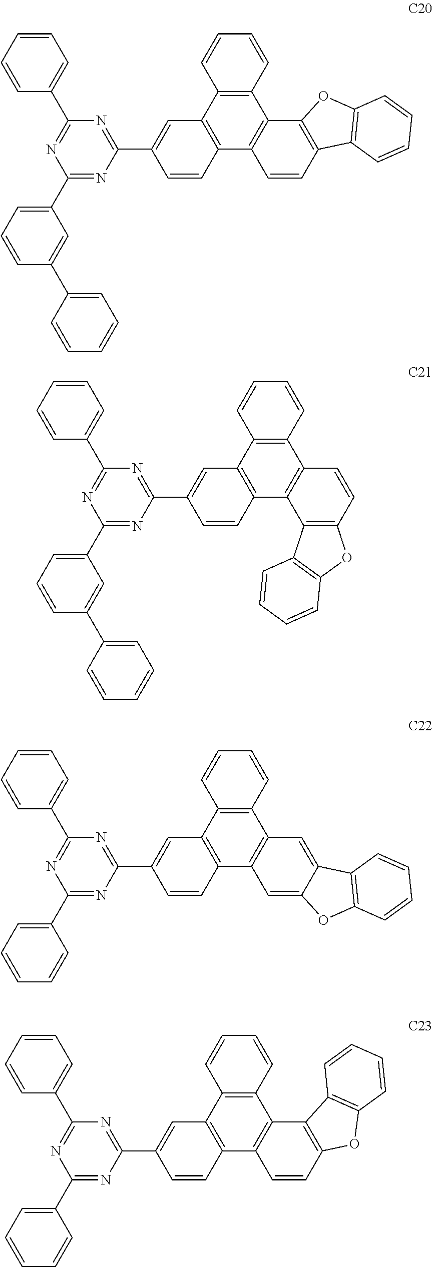

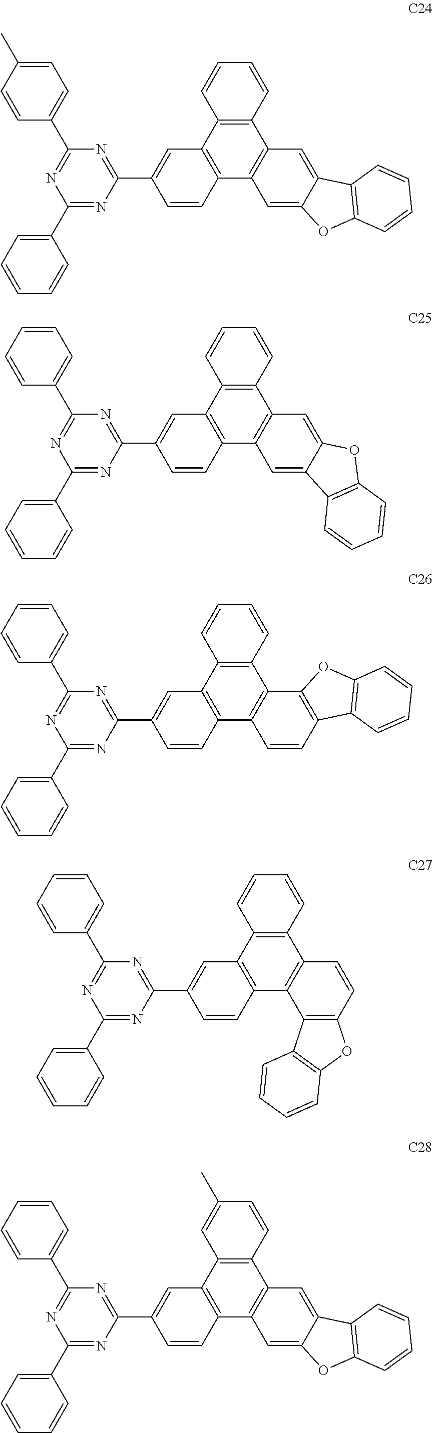

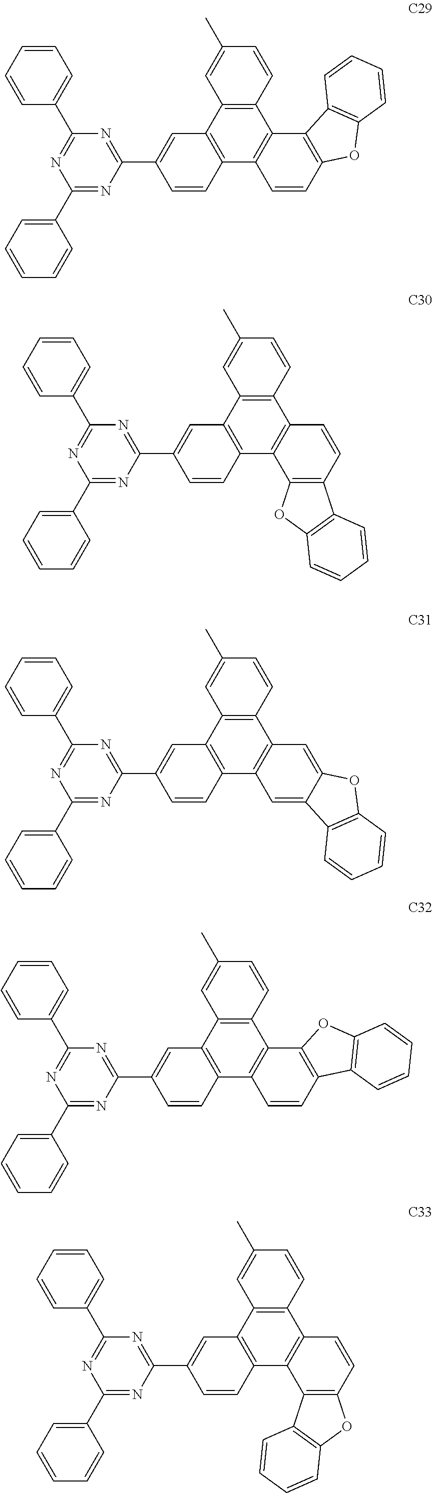 [SMILES: Cc1ccc2c(c1)c1cc(-c3nc(-c4ccccc4)nc(-c4ccccc4)n3)ccc1c1ccc3oc4ccccc4c3c12 ; Cc1ccc2c(c1)c1cc(-c3nc(-c4ccccc4)nc(-c4ccccc4)n3)ccc1c1c2ccc2c3ccccc3oc21 ; Cc1ccc2c(c1)c1cc(-c3nc(-c4ccccc4)nc(-c4ccccc4)n3)ccc1c1cc3c(cc21)oc1ccccc13 ; Cc1ccc2c(c1)c1cc(-c3nc(-c4ccccc4)nc(-c4ccccc4)n3)ccc1c1ccc3c4ccccc4oc3c12 ; Cc1ccc2c(c1)c1cc(-c3nc(-c4ccccc4)nc(-c4ccccc4)n3)ccc1c1c2ccc2oc3ccccc3c21]

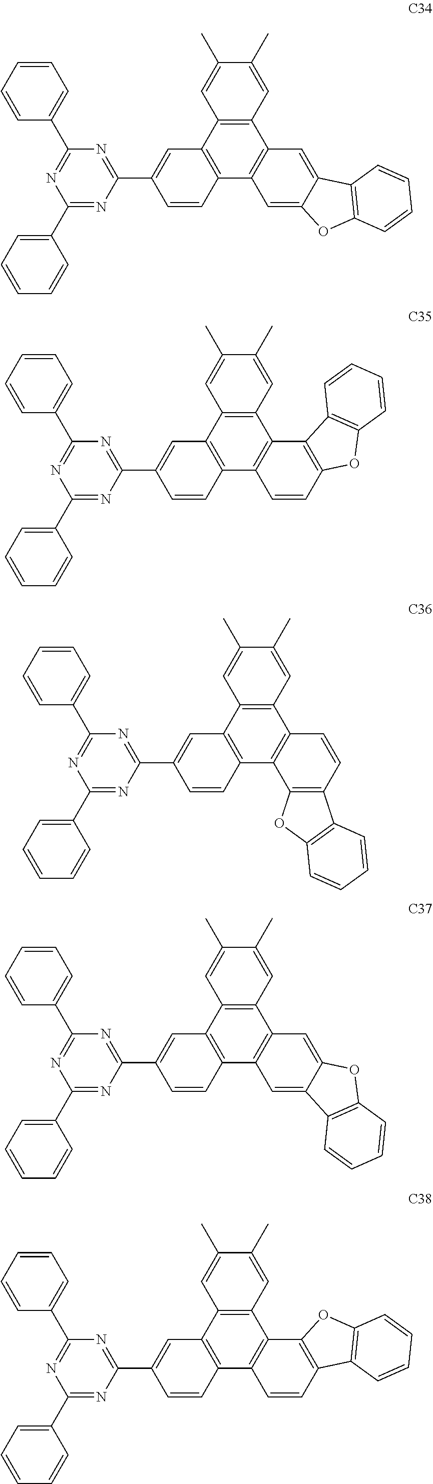 [SMILES: Cc1cc2c3cc(-c4nc(-c5ccccc5)nc(-c5ccccc5)n4)ccc3c3cc4oc5ccccc5c4cc3c2cc1C ; Cc1cc2c3cc(-c4nc(-c5ccccc5)nc(-c5ccccc5)n4)ccc3c3ccc4oc5ccccc5c4c3c2cc1C ; Cc1cc2c3cc(-c4nc(-c5ccccc5)nc(-c5ccccc5)n4)ccc3c3c(ccc4c5ccccc5oc43)c2cc1C ; Cc1cc2c3cc(-c4nc(-c5ccccc5)nc(-c5ccccc5)n4)ccc3c3cc4c(cc3c2cc1C)oc1ccccc14 ; Cc1cc2c3cc(-c4nc(-c5ccccc5)nc(-c5ccccc5)n4)ccc3c3ccc4c5ccccc5oc4c3c2cc1C]

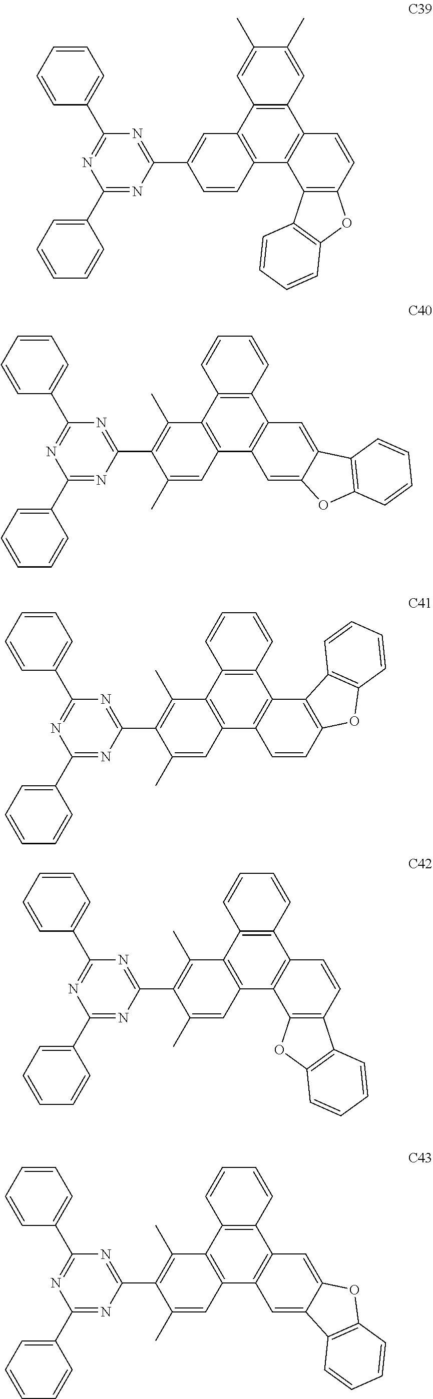

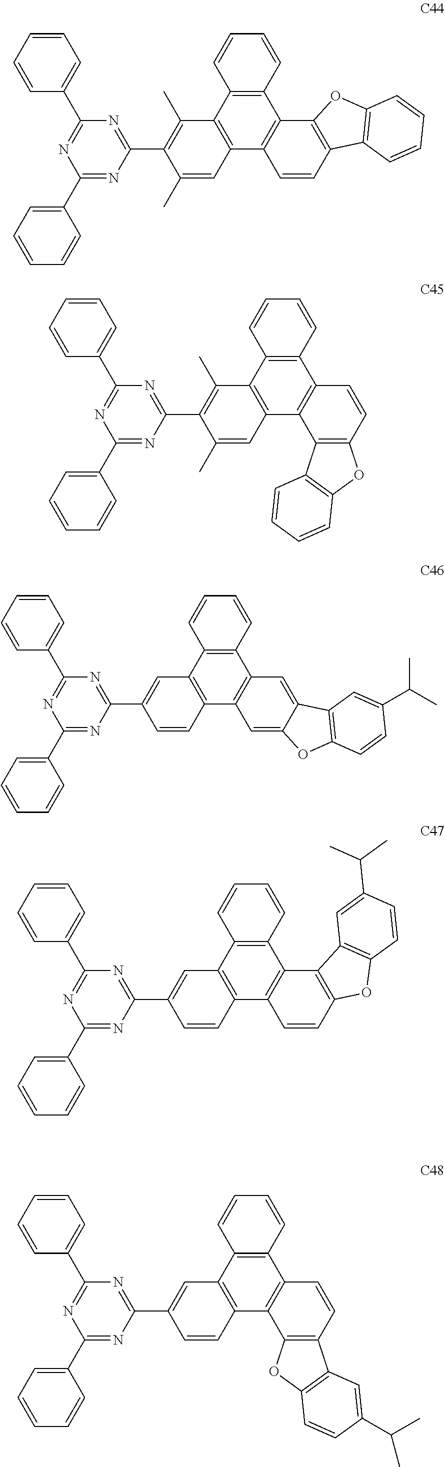

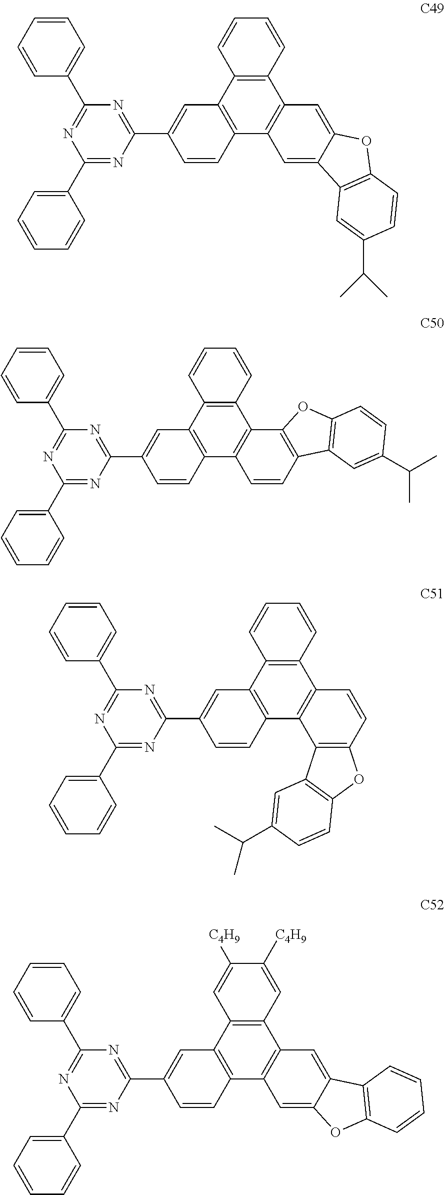

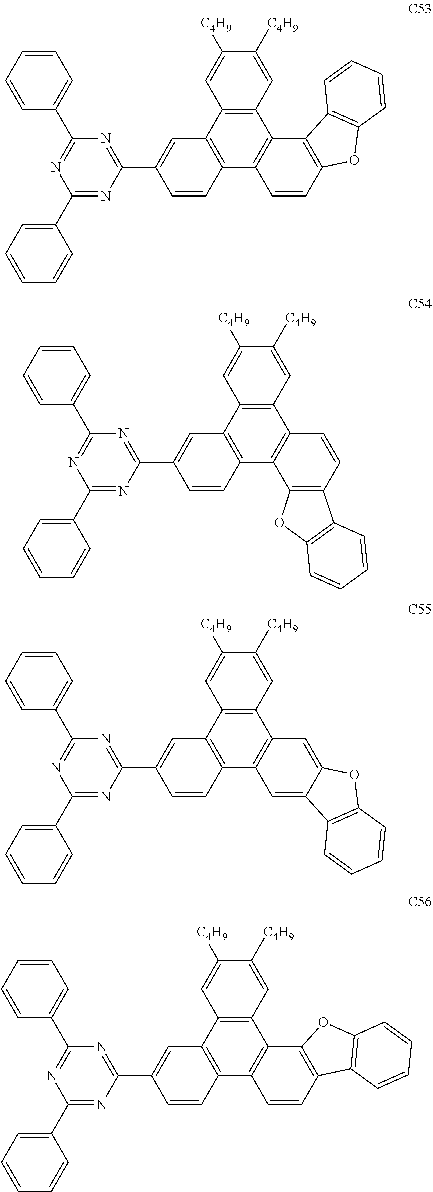

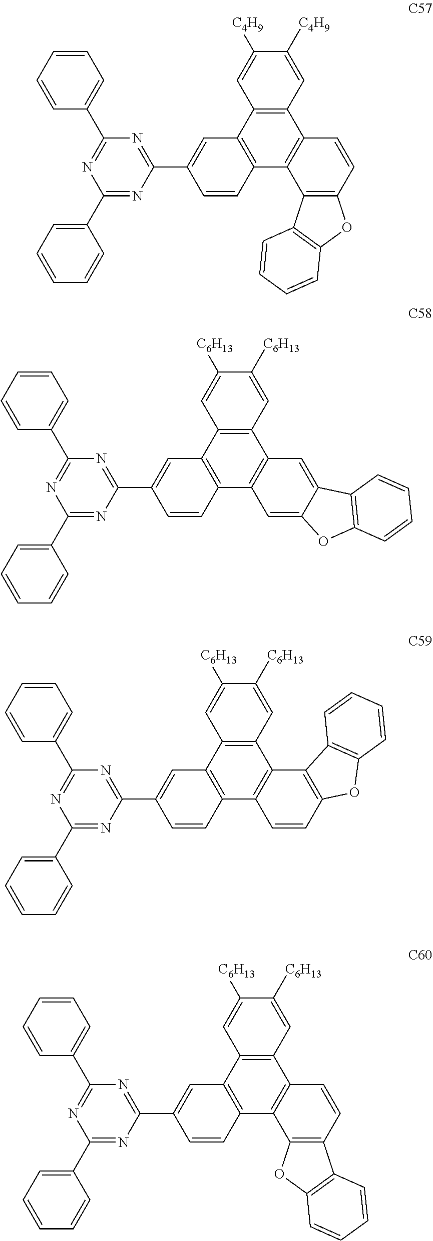

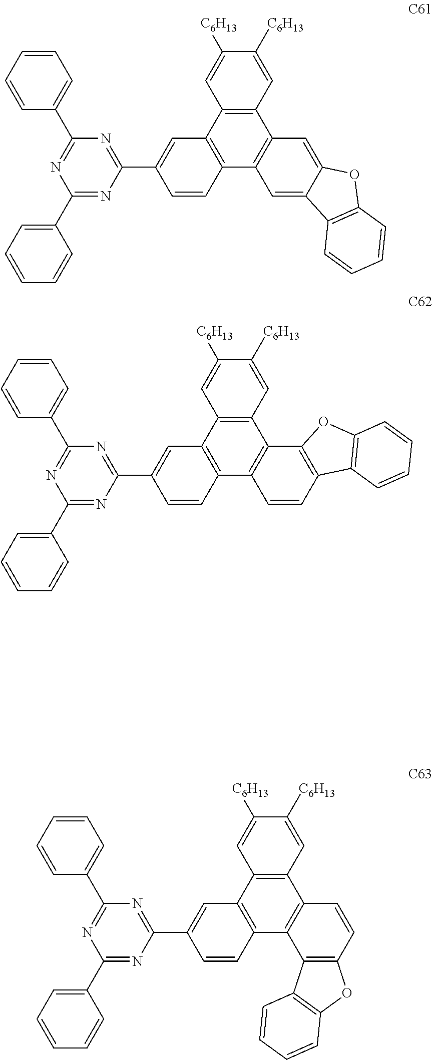

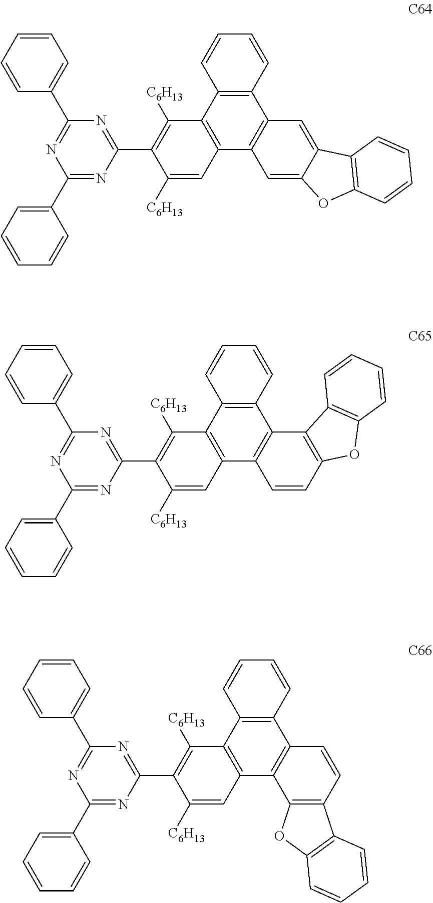

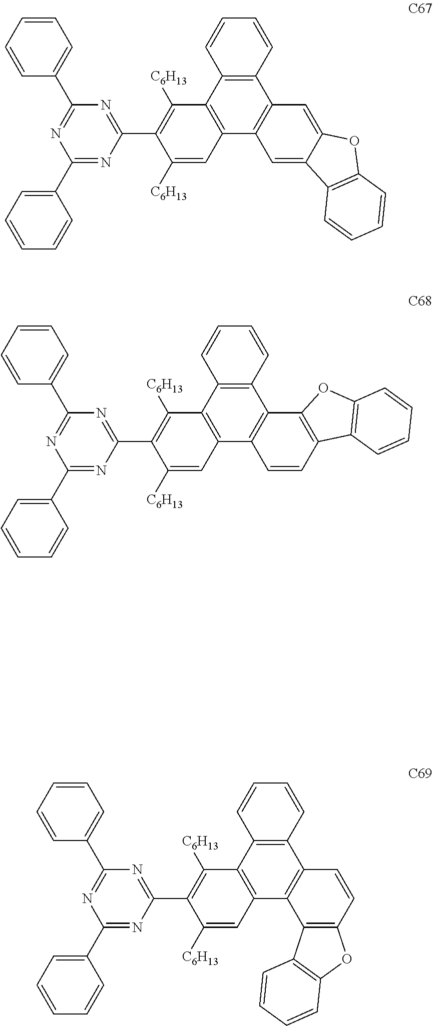

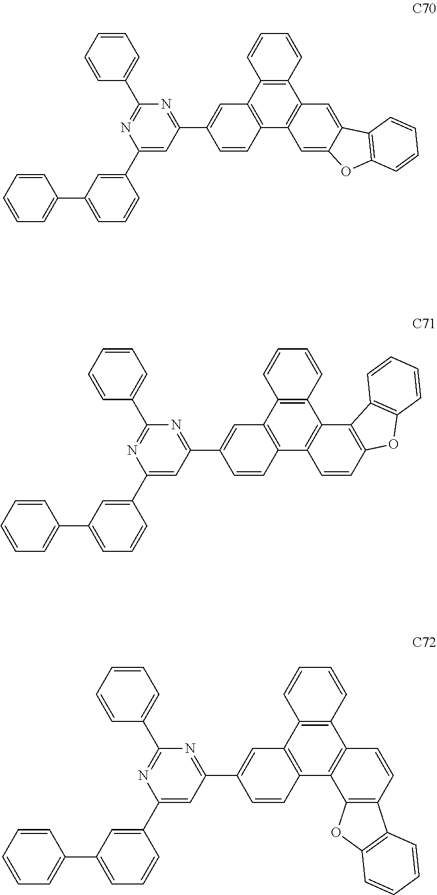 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4ccc6c7ccccc7oc6c45)nc(-c4ccccc4)n3)c2)cc1]

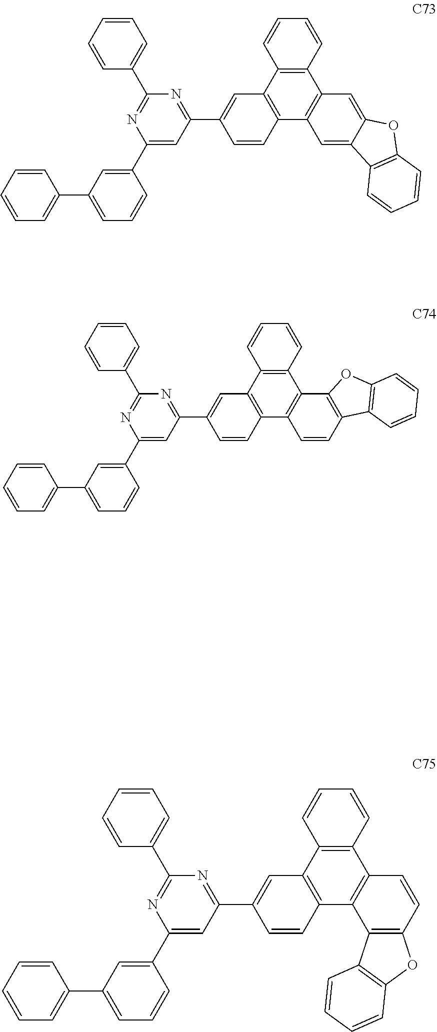 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

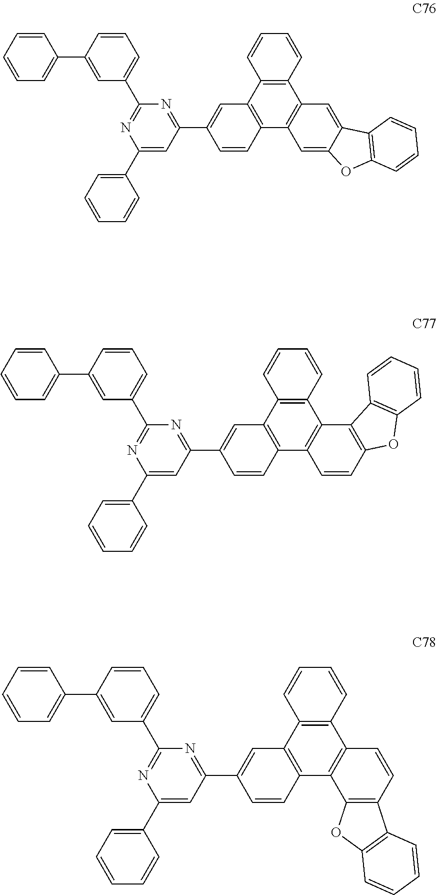 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

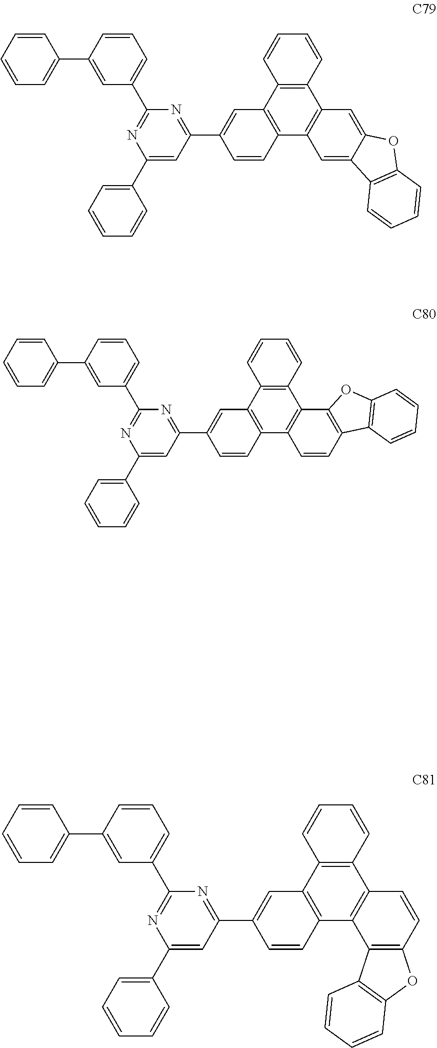 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

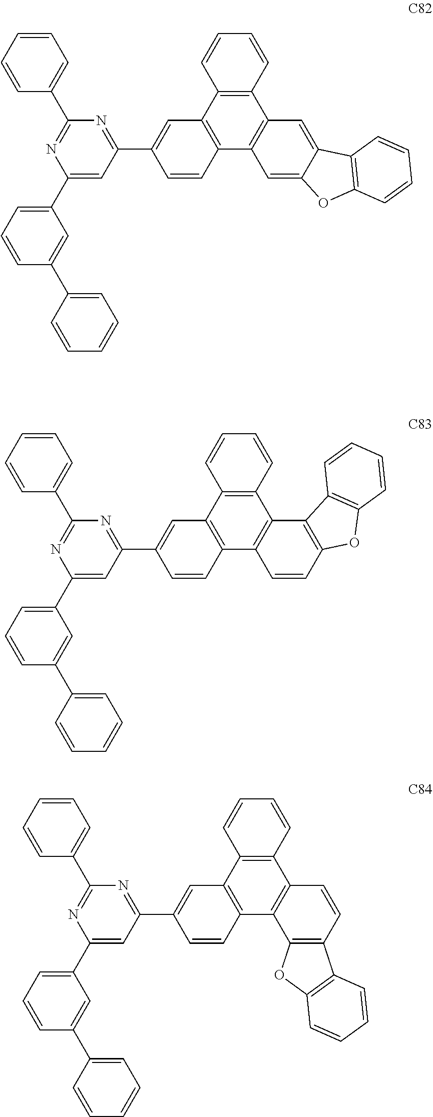 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4ccc6c7ccccc7oc6c45)nc(-c4ccccc4)n3)c2)cc1]

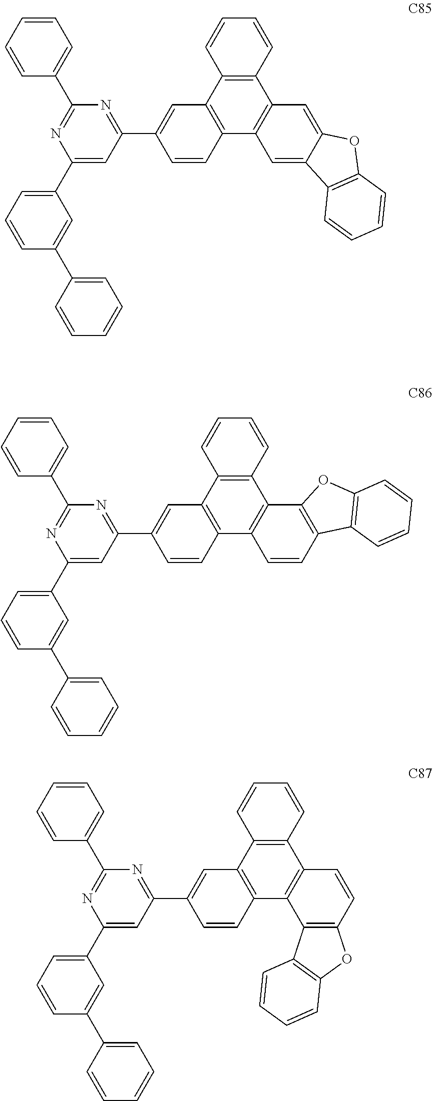 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

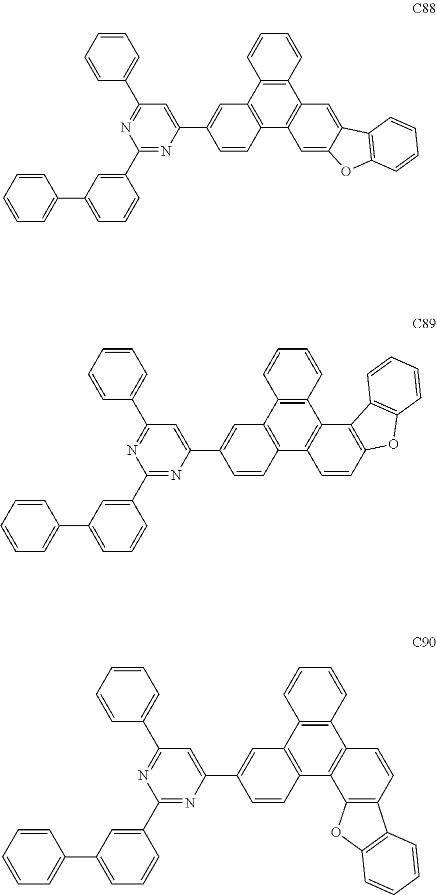 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

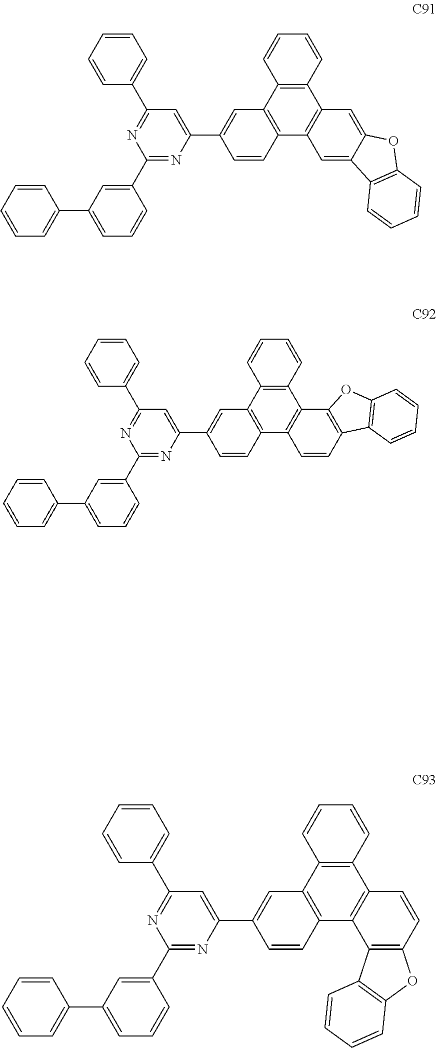 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

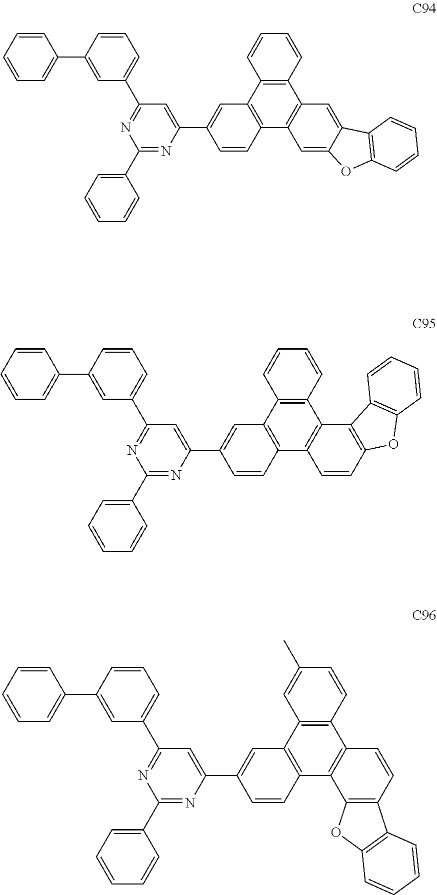 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; Cc1ccc2c(c1)c1cc(-c3cc(-c4cccc(-c5ccccc5)c4)nc(-c4ccccc4)n3)ccc1c1c2ccc2c3ccccc3oc21]

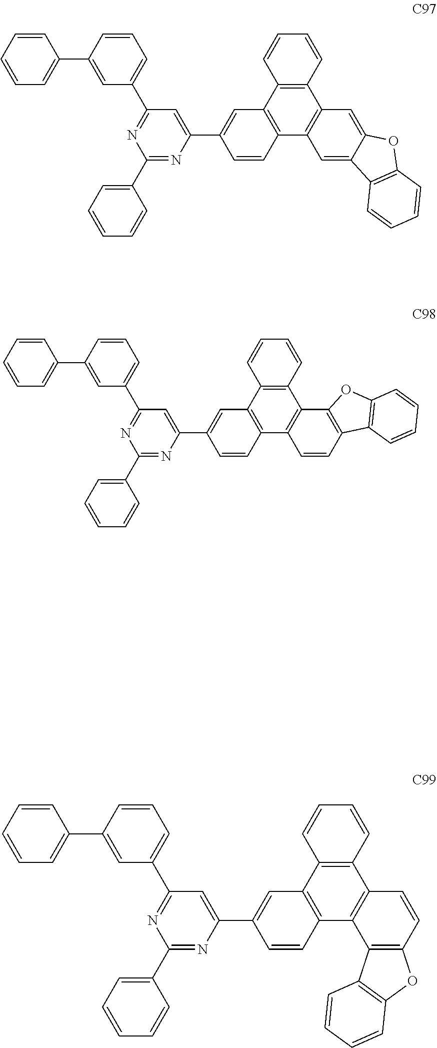 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

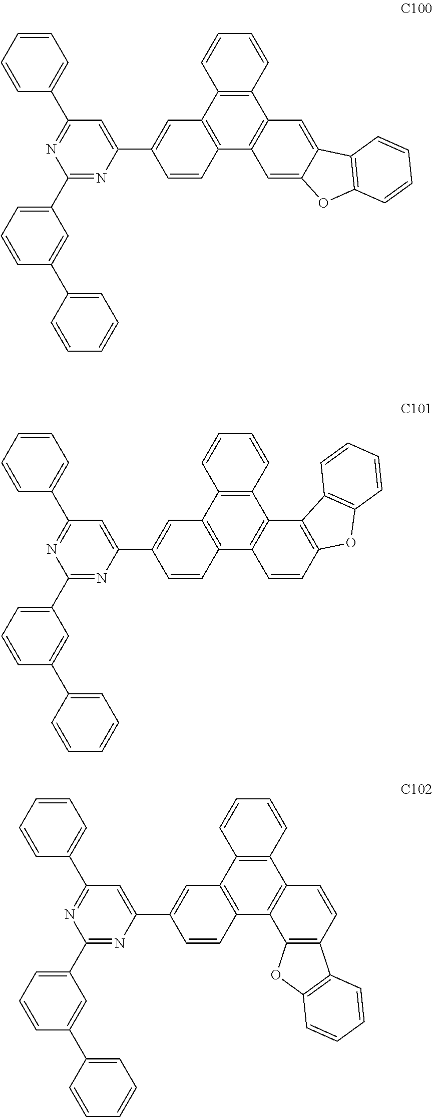 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

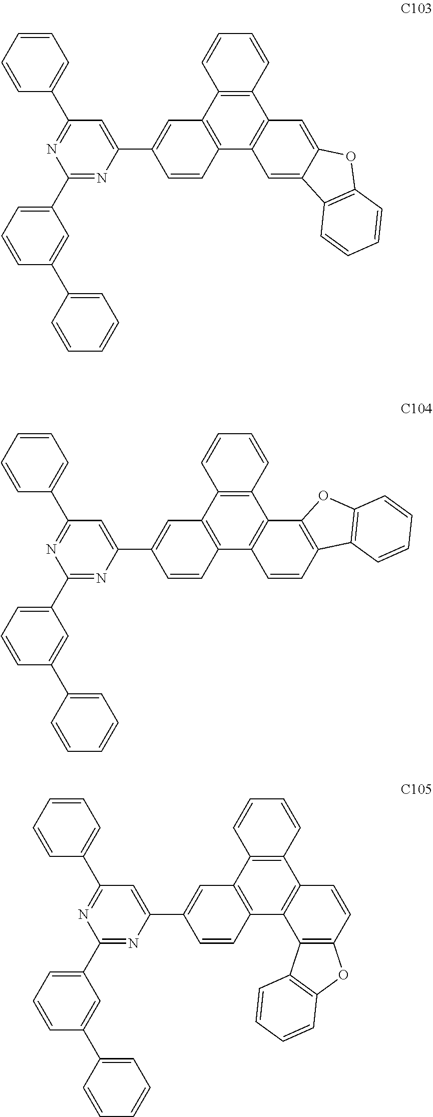 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

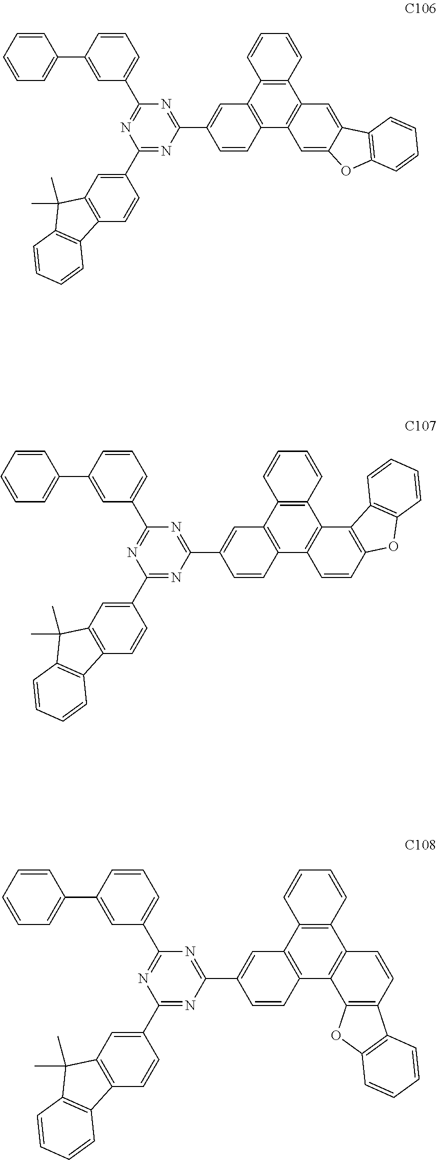 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4c5ccc5oc6ccccc6c54)n3)cc21 ; CC1(C)c2ccccc2-c2ccc(-c3nc(-c4cccc(-c5ccccc5)c4)nc(-c4ccc5c(c4)c4ccccc4c4ccc6c7ccccc7oc6c45)n3)cc21]

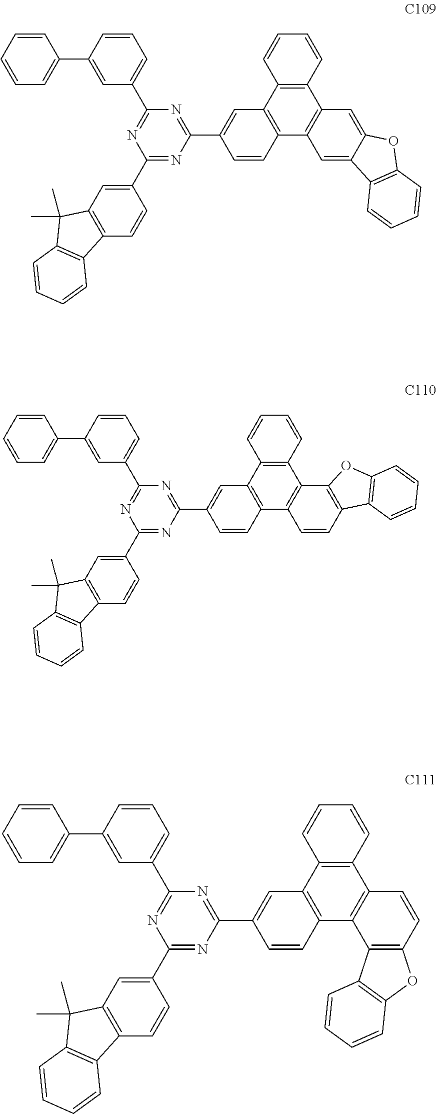 [SMILES: CC1(C)c2ccccc2-c2ccc(-c3nc(-c4cccc(-c5ccccc5)c4)nc(-c4ccc5c(c4)c4ccccc4c4cc6oc7ccccc7c6cc54)n3)cc21 ; CC1(C)c2ccccc2-c2ccc(-c3nc(-c4cccc(-c5ccccc5)c4)nc(-c4ccc5c(c4)c4ccccc4c4c5ccc5c6ccccc6oc54)n3)cc21 ; CC1(C)c2ccccc2-c2ccc(-c3nc(-c4cccc(-c5ccccc5)c4)nc(-c4ccc5c(c4)c4ccccc4c4ccc6oc7ccccc7c6c45)n3)cc21]

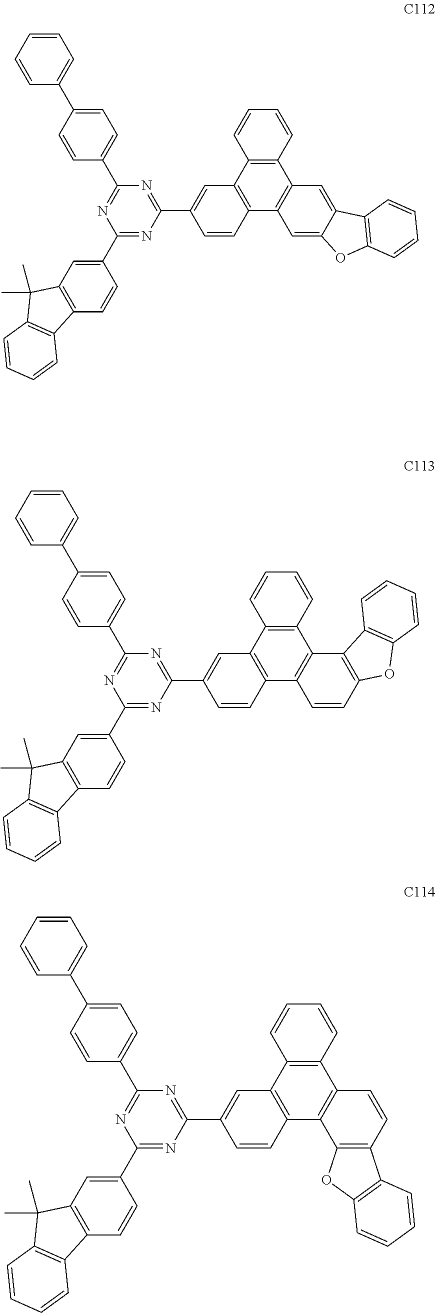 [SMILES: CC1(C)c2ccccc2-c2ccc(-c3nc(-c4ccc(-c5ccccc5)cc4)nc(-c4ccc5c(c4)c4ccccc4c4cc6c(cc54)oc4ccccc46)n3)cc21 ; CC1(C)c2ccccc2-c2ccc(-c3nc(-c4ccc(-c5ccccc5)cc4)nc(-c4ccc5c(c4)c4ccccc4c4c5ccc5oc6ccccc6c54)n3)cc21 ; CC1(C)c2ccccc2-c2ccc(-c3nc(-c4ccc(-c5ccccc5)cc4)nc(-c4ccc5c(c4)c4ccccc4c4ccc6c7ccccc7oc6c45)n3)cc21]

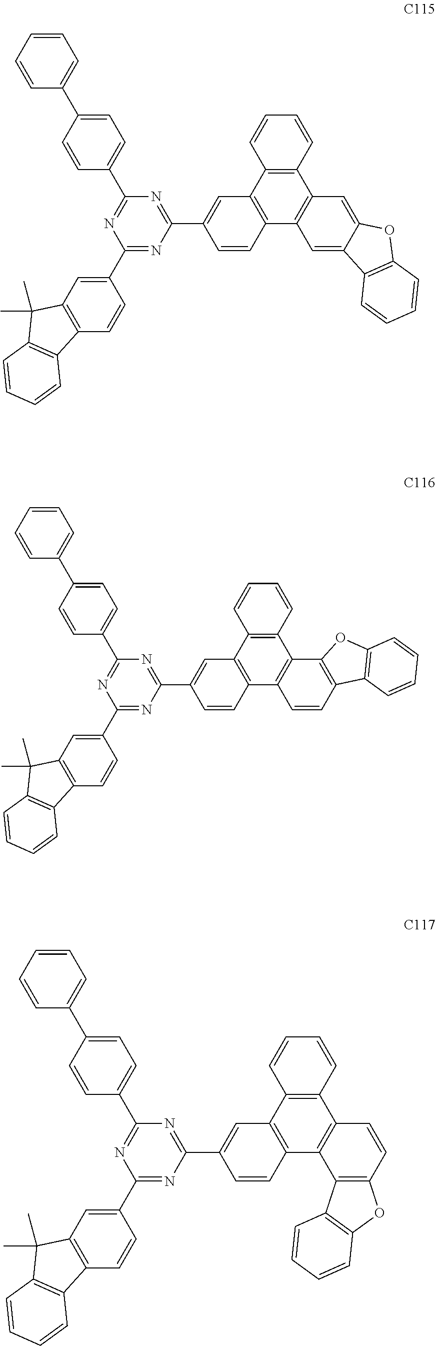 [SMILES: CC1(C)c2ccccc2-c2ccc(-c3nc(-c4ccc(-c5ccccc5)cc4)nc(-c4ccc5c(c4)c4ccccc4c4cc6oc7ccccc7c6cc54)n3)cc21 ; CC1(C)c2ccccc2-c2ccc(-c3nc(-c4ccc(-c5ccccc5)cc4)nc(-c4ccc5c(c4)c4ccccc4c4c5ccc5c6ccccc6oc54)n3)cc21 ; CC1(C)c2ccccc2-c2ccc(-c3nc(-c4ccc(-c5ccccc5)cc4)nc(-c4ccc5c(c4)c4ccccc4c4ccc6oc7ccccc7c6c45)n3)cc21]

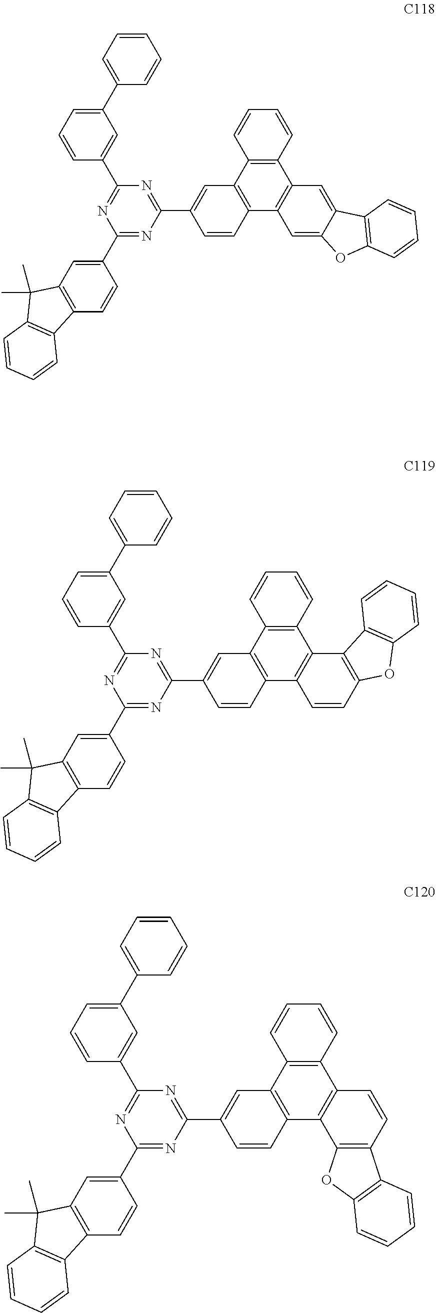 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4c5ccc5oc6ccccc6c54)n3)cc21 ; CC1(C)c2ccccc2-c2ccc(-c3nc(-c4cccc(-c5ccccc5)c4)nc(-c4ccc5c(c4)c4ccccc4c4ccc6c7ccccc7oc6c45)n3)cc21]

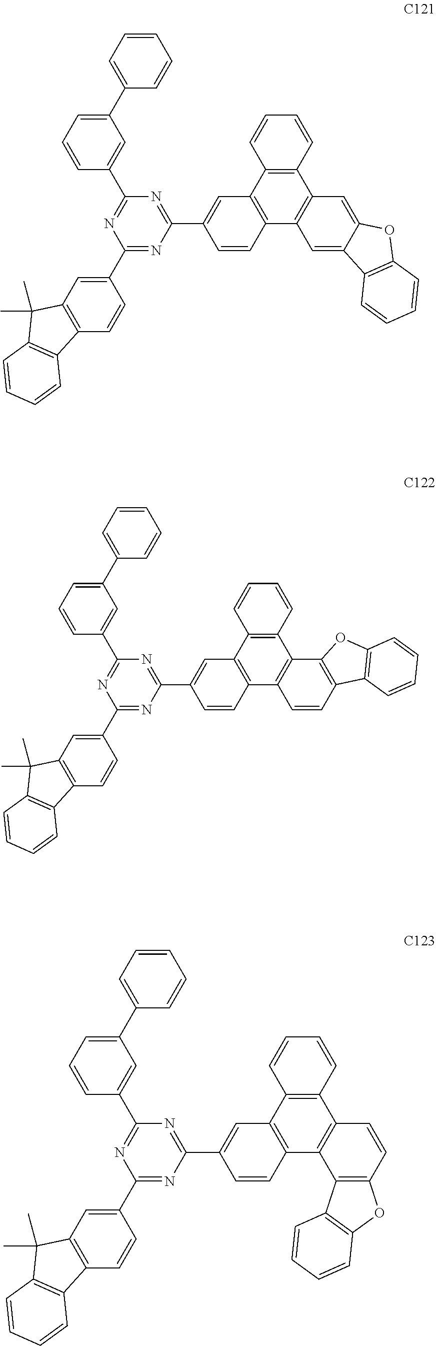 [SMILES: CC1(C)c2ccccc2-c2ccc(-c3nc(-c4cccc(-c5ccccc5)c4)nc(-c4ccc5c(c4)c4ccccc4c4cc6oc7ccccc7c6cc54)n3)cc21 ; CC1(C)c2ccccc2-c2ccc(-c3nc(-c4cccc(-c5ccccc5)c4)nc(-c4ccc5c(c4)c4ccccc4c4c5ccc5c6ccccc6oc54)n3)cc21 ; CC1(C)c2ccccc2-c2ccc(-c3nc(-c4cccc(-c5ccccc5)c4)nc(-c4ccc5c(c4)c4ccccc4c4ccc6oc7ccccc7c6c45)n3)cc21]

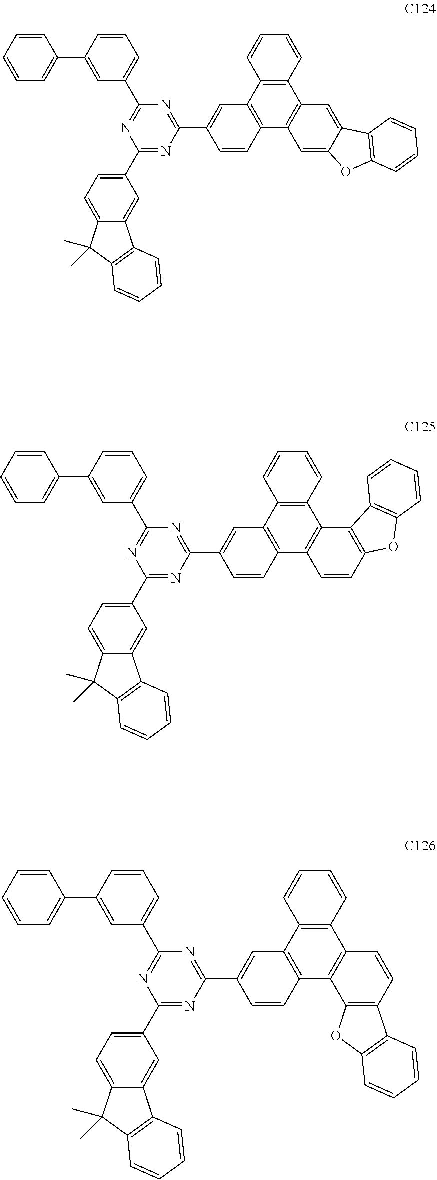 [SMILES: CC1(C)c2ccccc2-c2cc(-c3nc(-c4cccc(-c5ccccc5)c4)nc(-c4ccc5c(c4)c4ccccc4c4cc6c(cc54)oc4ccccc46)n3)ccc21 ; CC1(C)c2ccccc2-c2cc(-c3nc(-c4cccc(-c5ccccc5)c4)nc(-c4ccc5c(c4)c4ccccc4c4c5ccc5oc6ccccc6c54)n3)ccc21 ; CC1(C)c2ccccc2-c2cc(-c3nc(-c4cccc(-c5ccccc5)c4)nc(-c4ccc5c(c4)c4ccccc4c4ccc6c7ccccc7oc6c45)n3)ccc21]

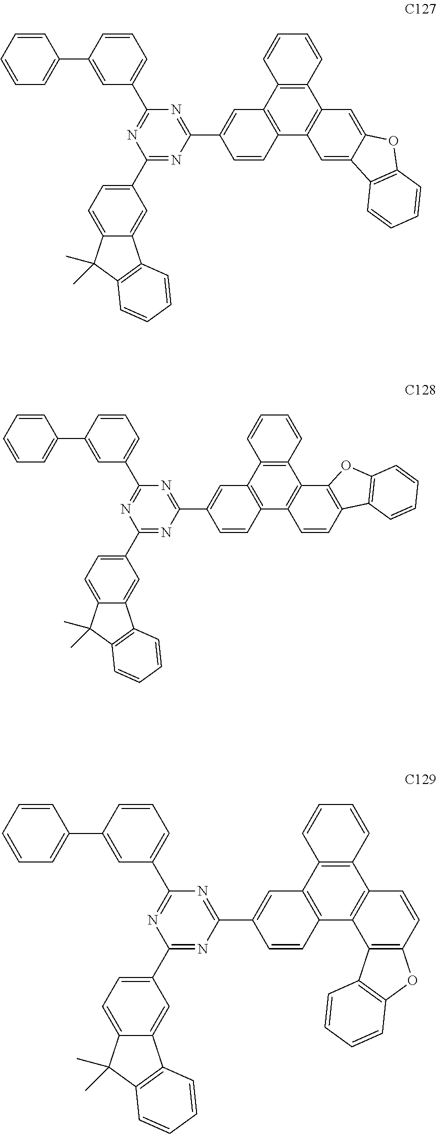 [SMILES: CC1(C)c2ccccc2-c2cc(-c3nc(-c4cccc(-c5ccccc5)c4)nc(-c4ccc5c(c4)c4ccccc4c4cc6oc7ccccc7c6cc54)n3)ccc21 ; CC1(C)c2ccccc2-c2cc(-c3nc(-c4cccc(-c5ccccc5)c4)nc(-c4ccc5c(c4)c4ccccc4c4c5ccc5c6ccccc6oc54)n3)ccc21 ; CC1(C)c2ccccc2-c2cc(-c3nc(-c4cccc(-c5ccccc5)c4)nc(-c4ccc5c(c4)c4ccccc4c4ccc6oc7ccccc7c6c45)n3)ccc21]

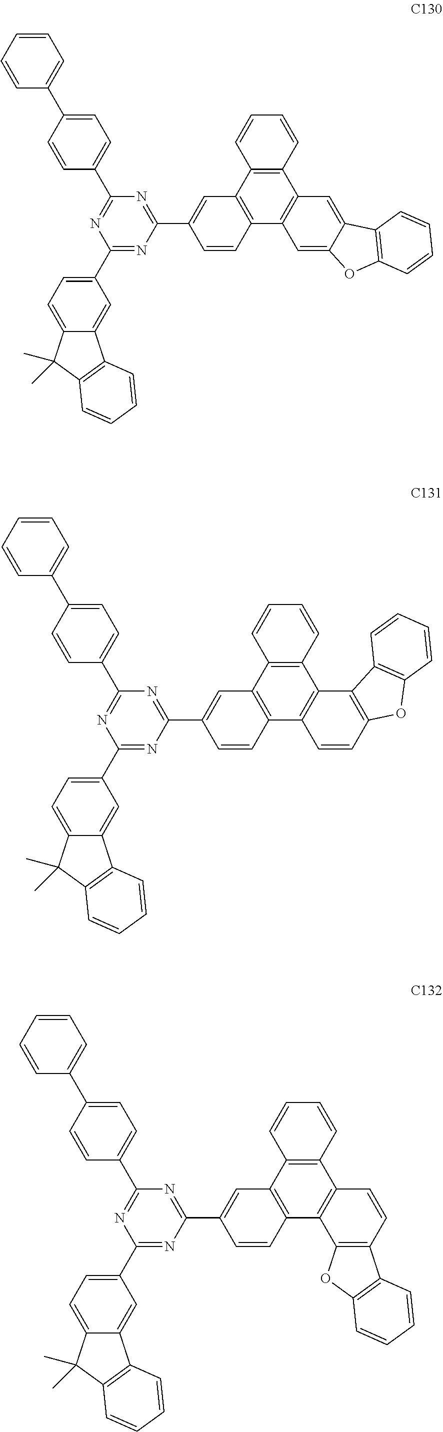 [SMILES: CC1(C)c2ccccc2-c2cc(-c3nc(-c4ccc(-c5ccccc5)cc4)nc(-c4ccc5c(c4)c4ccccc4c4cc6c(cc54)oc4ccccc46)n3)ccc21 ; CC1(C)c2ccccc2-c2cc(-c3nc(-c4ccc(-c5ccccc5)cc4)nc(-c4ccc5c(c4)c4ccccc4c4c5ccc5oc6ccccc6c54)n3)ccc21 ; CC1(C)c2ccccc2-c2cc(-c3nc(-c4ccc(-c5ccccc5)cc4)nc(-c4ccc5c(c4)c4ccccc4c4ccc6c7ccccc7oc6c45)n3)ccc21]

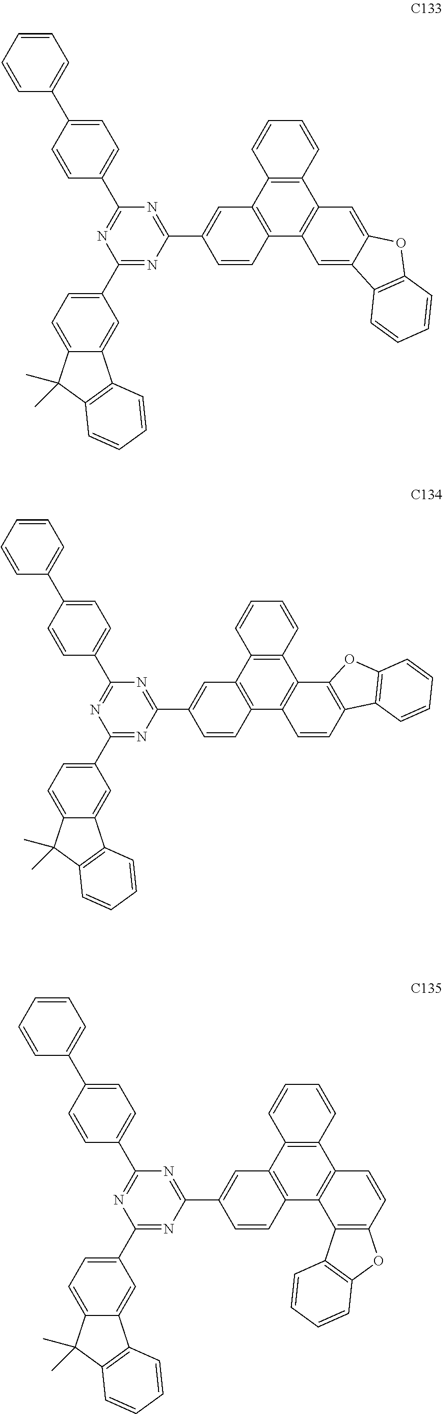 [SMILES: CC1(C)c2ccccc2-c2cc(-c3nc(-c4ccc(-c5ccccc5)cc4)nc(-c4ccc5c(c4)c4ccccc4c4cc6oc7ccccc7c6cc54)n3)ccc21 ; CC1(C)c2ccccc2-c2cc(-c3nc(-c4ccc(-c5ccccc5)cc4)nc(-c4ccc5c(c4)c4ccccc4c4c5ccc5c6ccccc6oc54)n3)ccc21 ; CC1(C)c2ccccc2-c2cc(-c3nc(-c4ccc(-c5ccccc5)cc4)nc(-c4ccc5c(c4)c4ccccc4c4ccc6oc7ccccc7c6c45)n3)ccc21]

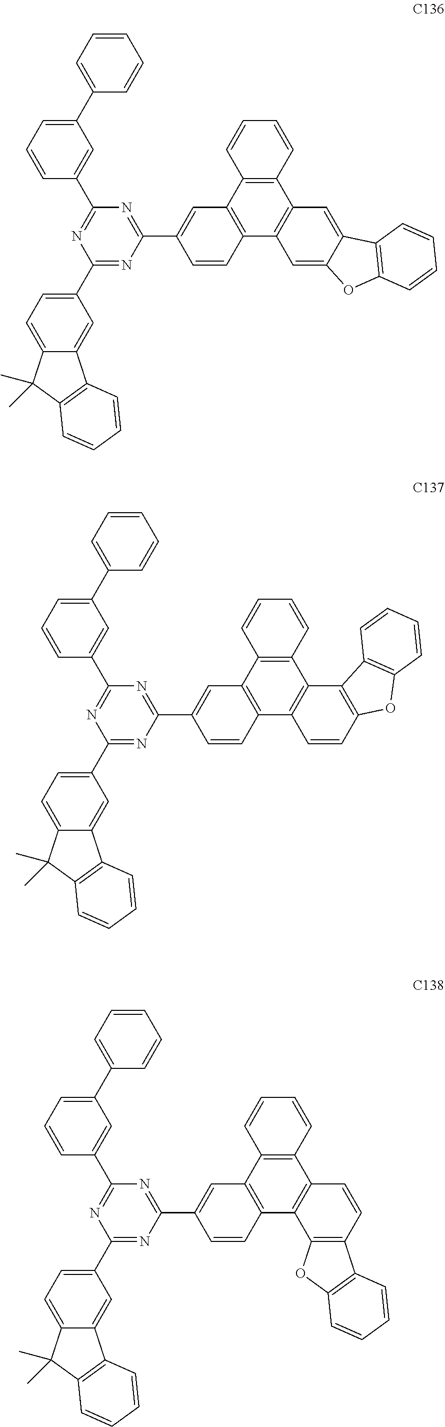 [SMILES: CC1(C)c2ccccc2-c2cc(-c3nc(-c4cccc(-c5ccccc5)c4)nc(-c4ccc5c(c4)c4ccccc4c4cc6c(cc54)oc4ccccc46)n3)ccc21 ; CC1(C)c2ccccc2-c2cc(-c3nc(-c4cccc(-c5ccccc5)c4)nc(-c4ccc5c(c4)c4ccccc4c4c5ccc5oc6ccccc6c54)n3)ccc21 ; CC1(C)c2ccccc2-c2cc(-c3nc(-c4cccc(-c5ccccc5)c4)nc(-c4ccc5c(c4)c4ccccc4c4ccc6c7ccccc7oc6c45)n3)ccc21]

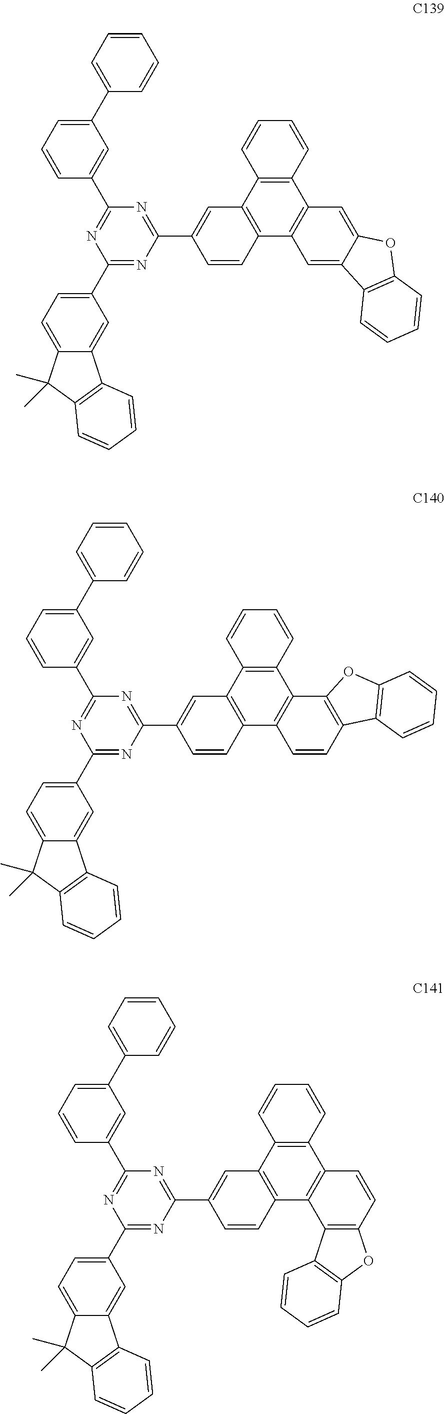 [SMILES: CC1(C)c2ccccc2-c2cc(-c3nc(-c4cccc(-c5ccccc5)c4)nc(-c4ccc5c(c4)c4ccccc4c4cc6oc7ccccc7c6cc54)n3)ccc21 ; CC1(C)c2ccccc2-c2cc(-c3nc(-c4cccc(-c5ccccc5)c4)nc(-c4ccc5c(c4)c4ccccc4c4c5ccc5c6ccccc6oc54)n3)ccc21 ; CC1(C)c2ccccc2-c2cc(-c3nc(-c4cccc(-c5ccccc5)c4)nc(-c4ccc5c(c4)c4ccccc4c4ccc6oc7ccccc7c6c45)n3)ccc21]

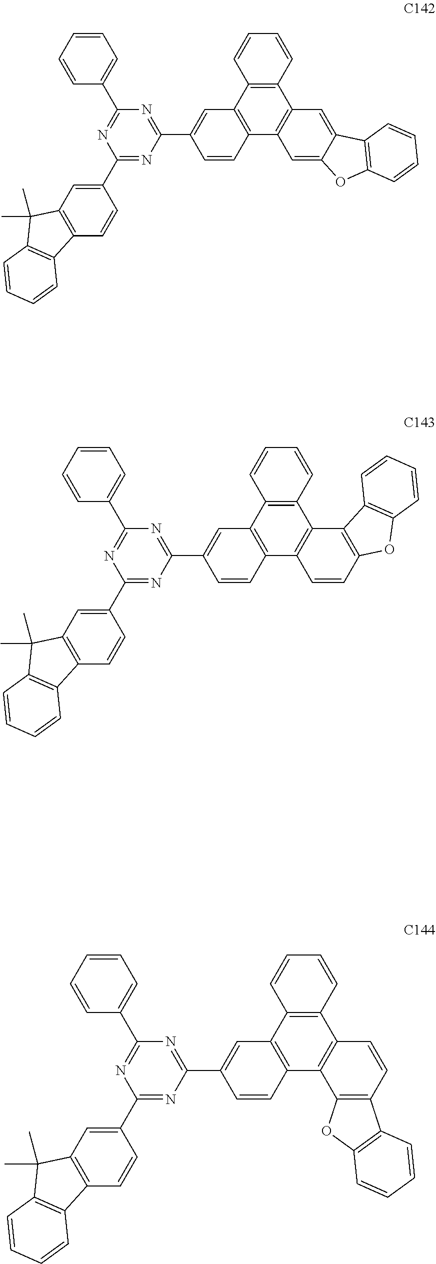 [SMILES: CC1(C)c2ccccc2-c2ccc(-c3nc(-c4ccccc4)nc(-c4ccc5c(c4)c4ccccc4c4cc6c(cc54)oc4ccccc46)n3)cc21 ; CC1(C)c2ccccc2-c2ccc(-c3nc(-c4ccccc4)nc(-c4ccc5c(c4)c4ccccc4c4c5ccc5oc6ccccc6c54)n3)cc21 ; CC1(C)c2ccccc2-c2ccc(-c3nc(-c4ccccc4)nc(-c4ccc5c(c4)c4ccccc4c4ccc6c7ccccc7oc6c45)n3)cc21]

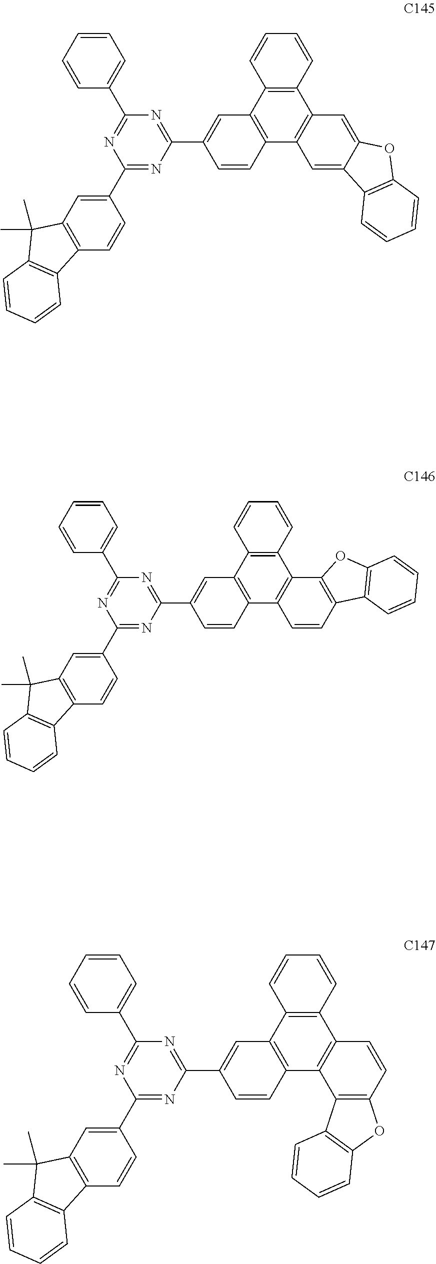 [SMILES: CC1(C)c2ccccc2-c2ccc(-c3nc(-c4ccccc4)nc(-c4ccc5c(c4)c4ccccc4c4cc6oc7ccccc7c6cc54)n3)cc21 ; CC1(C)c2ccccc2-c2ccc(-c3nc(-c4ccccc4)nc(-c4ccc5c(c4)c4ccccc4c4c5ccc5c6ccccc6oc54)n3)cc21 ; CC1(C)c2ccccc2-c2ccc(-c3nc(-c4ccccc4)nc(-c4ccc5c(c4)c4ccccc4c4ccc6oc7ccccc7c6c45)n3)cc21]

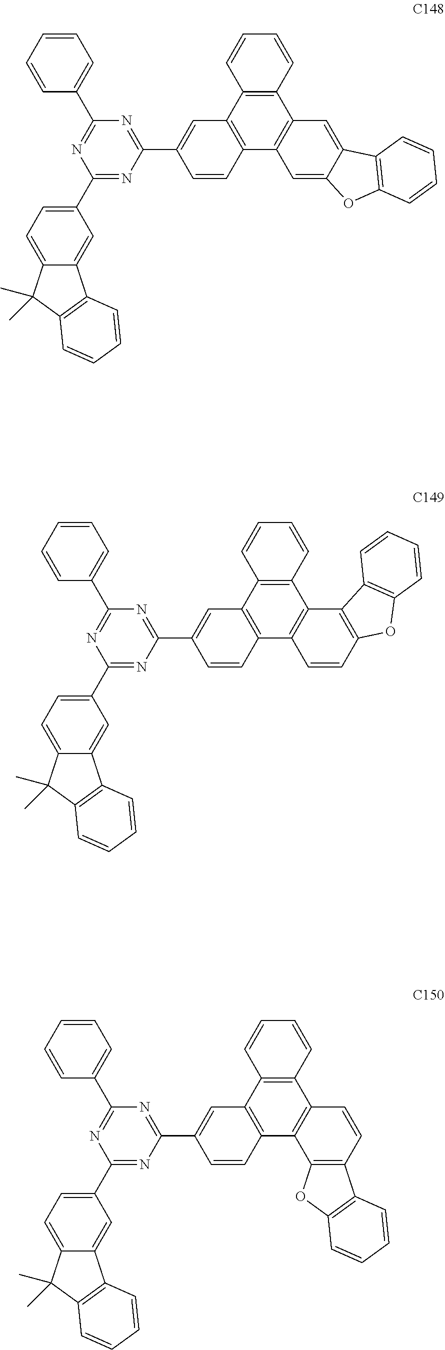 [SMILES: CC1(C)c2ccccc2-c2cc(-c3nc(-c4ccccc4)nc(-c4ccc5c(c4)c4ccccc4c4cc6c(cc54)oc4ccccc46)n3)ccc21 ; CC1(C)c2ccccc2-c2cc(-c3nc(-c4ccccc4)nc(-c4ccc5c(c4)c4ccccc4c4c5ccc5oc6ccccc6c54)n3)ccc21 ; CC1(C)c2ccccc2-c2cc(-c3nc(-c4ccccc4)nc(-c4ccc5c(c4)c4ccccc4c4ccc6c7ccccc7oc6c45)n3)ccc21]

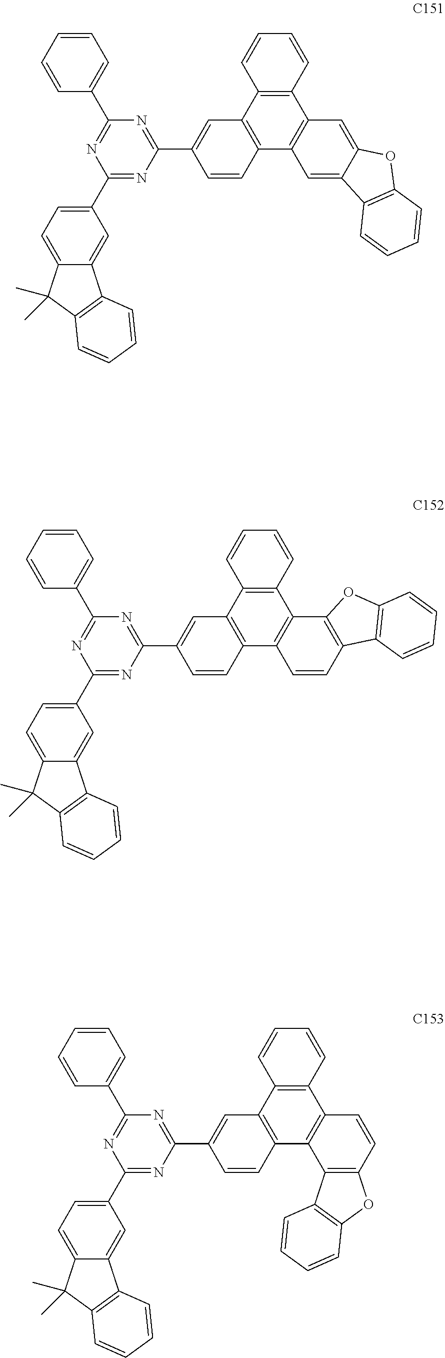 [SMILES: CC1(C)c2ccccc2-c2cc(-c3nc(-c4ccccc4)nc(-c4ccc5c(c4)c4ccccc4c4cc6oc7ccccc7c6cc54)n3)ccc21 ; CC1(C)c2ccccc2-c2cc(-c3nc(-c4ccccc4)nc(-c4ccc5c(c4)c4ccccc4c4c5ccc5c6ccccc6oc54)n3)ccc21 ; CC1(C)c2ccccc2-c2cc(-c3nc(-c4ccccc4)nc(-c4ccc5c(c4)c4ccccc4c4ccc6oc7ccccc7c6c45)n3)ccc21]

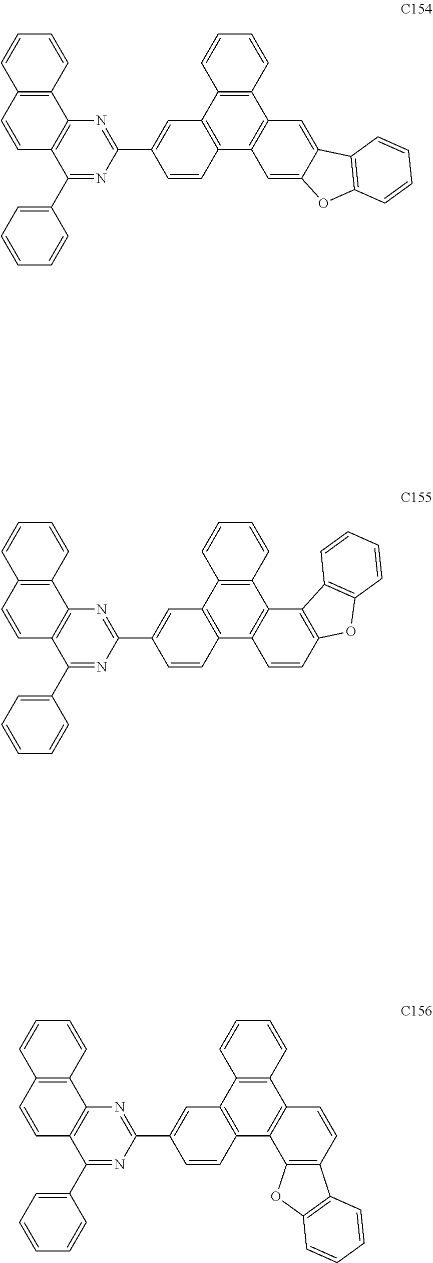 [SMILES: c1ccc(-c2nc(-c3ccc4c(c3)c3ccccc3c3cc5c(cc43)oc3ccccc35)nc3c2ccc2ccccc23)cc1 ; c1ccc(-c2nc(-c3ccc4c(c3)c3ccccc3c3c4ccc4oc5ccccc5c43)nc3c2ccc2ccccc23)cc1 ; c1ccc(-c2nc(-c3ccc4c(c3)c3ccccc3c3ccc5c6ccccc6oc5c34)nc3c2ccc2ccccc23)cc1]

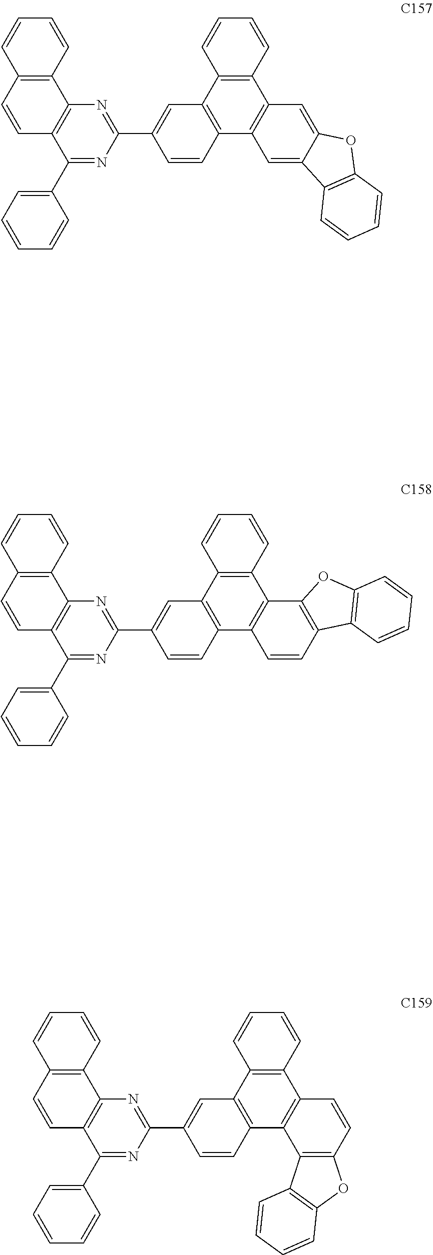 [SMILES: c1ccc(-c2nc(-c3ccc4c(c3)c3ccccc3c3cc5oc6ccccc6c5cc43)nc3c2ccc2ccccc23)cc1 ; c1ccc(-c2nc(-c3ccc4c(c3)c3ccccc3c3c4ccc4c5ccccc5oc43)nc3c2ccc2ccccc23)cc1 ; c1ccc(-c2nc(-c3ccc4c(c3)c3ccccc3c3ccc5oc6ccccc6c5c34)nc3c2ccc2ccccc23)cc1]

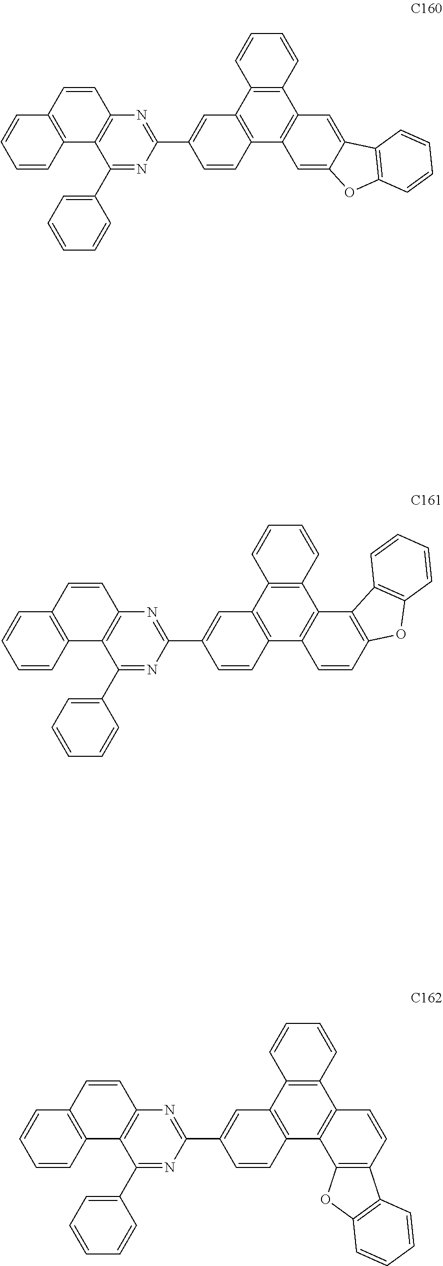 [SMILES: c1ccc(-c2nc(-c3ccc4c(c3)c3ccccc3c3cc5c(cc43)oc3ccccc35)nc3ccc4ccccc4c23)cc1 ; c1ccc(-c2nc(-c3ccc4c(c3)c3ccccc3c3c4ccc4oc5ccccc5c43)nc3ccc4ccccc4c23)cc1 ; c1ccc(-c2nc(-c3ccc4c(c3)c3ccccc3c3ccc5c6ccccc6oc5c34)nc3ccc4ccccc4c23)cc1]

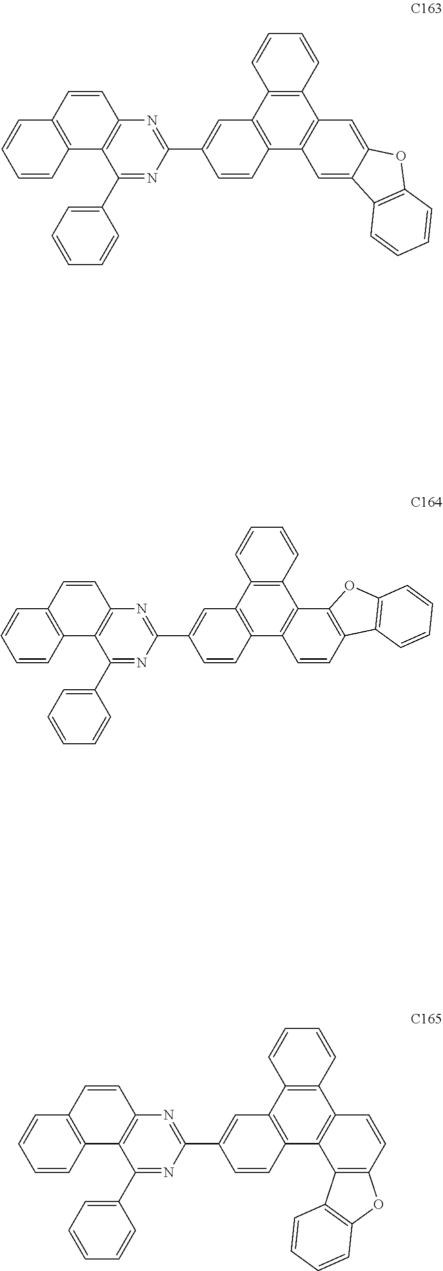 [SMILES: c1ccc(-c2nc(-c3ccc4c(c3)c3ccccc3c3cc5oc6ccccc6c5cc43)nc3ccc4ccccc4c23)cc1 ; c1ccc(-c2nc(-c3ccc4c(c3)c3ccccc3c3c4ccc4c5ccccc5oc43)nc3ccc4ccccc4c23)cc1 ; c1ccc(-c2nc(-c3ccc4c(c3)c3ccccc3c3ccc5oc6ccccc6c5c34)nc3ccc4ccccc4c23)cc1]

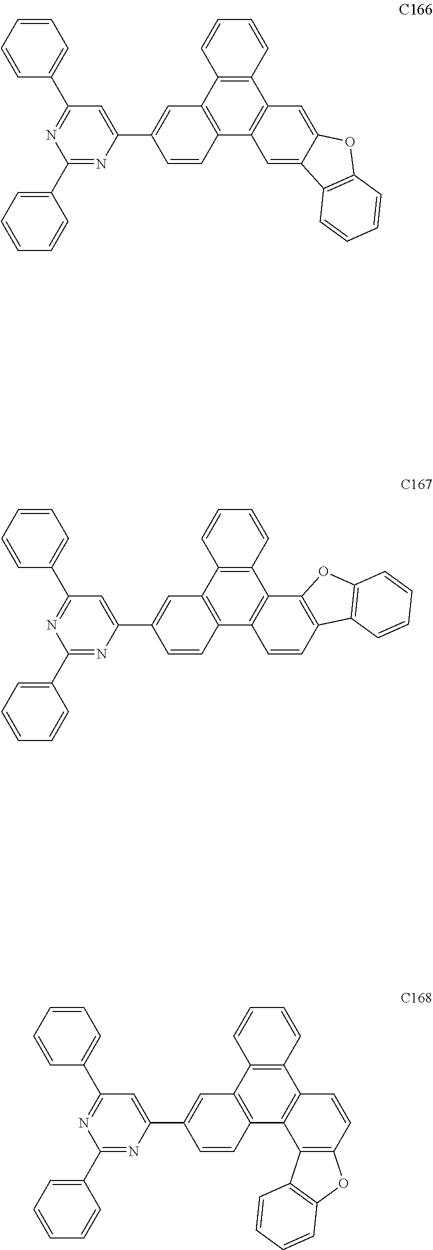 [SMILES: c1ccc(-c2cc(-c3ccc4c(c3)c3ccccc3c3cc5oc6ccccc6c5cc43)nc(-c3ccccc3)n2)cc1 ; c1ccc(-c2cc(-c3ccc4c(c3)c3ccccc3c3c4ccc4c5ccccc5oc43)nc(-c3ccccc3)n2)cc1 ; c1ccc(-c2cc(-c3ccc4c(c3)c3ccccc3c3ccc5oc6ccccc6c5c34)nc(-c3ccccc3)n2)cc1]

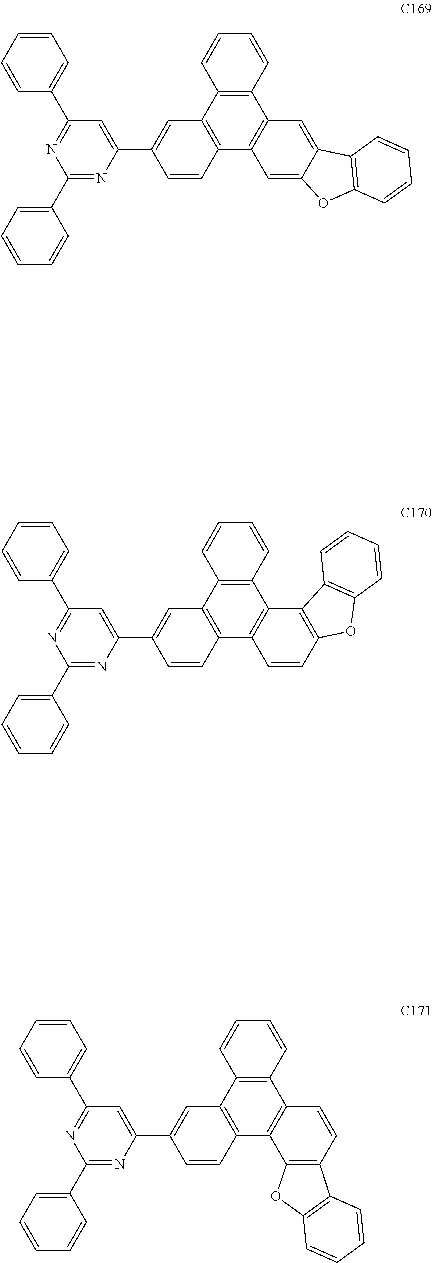 [SMILES: c1ccc(-c2cc(-c3ccc4c(c3)c3ccccc3c3cc5c(cc43)oc3ccccc35)nc(-c3ccccc3)n2)cc1 ; c1ccc(-c2cc(-c3ccc4c(c3)c3ccccc3c3c4ccc4oc5ccccc5c43)nc(-c3ccccc3)n2)cc1 ; c1ccc(-c2cc(-c3ccc4c(c3)c3ccccc3c3ccc5c6ccccc6oc5c34)nc(-c3ccccc3)n2)cc1]

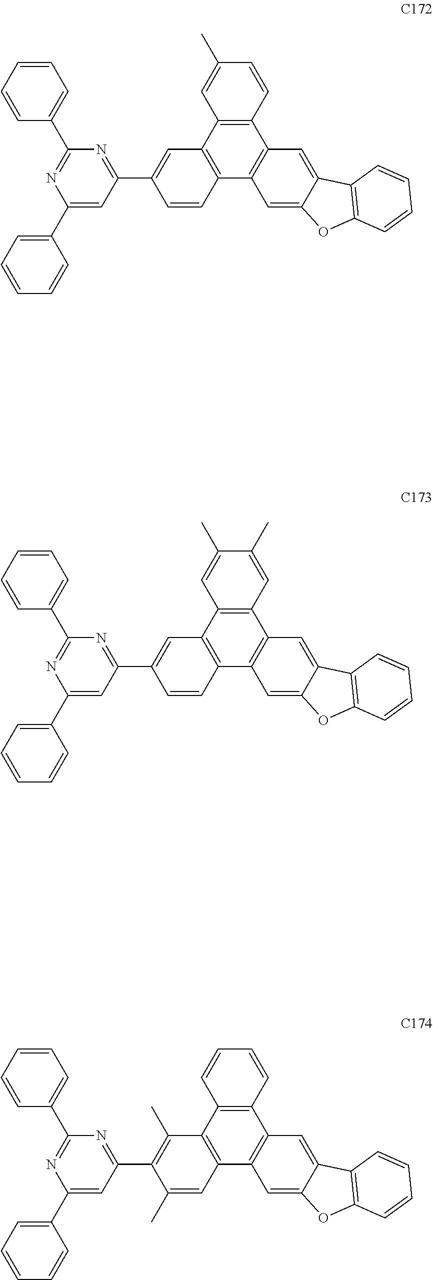 [SMILES: Cc1ccc2c(c1)c1cc(-c3cc(-c4ccccc4)nc(-c4ccccc4)n3)ccc1c1cc3oc4ccccc4c3cc21 ; Cc1cc2c3cc(-c4cc(-c5ccccc5)nc(-c5ccccc5)n4)ccc3c3cc4oc5ccccc5c4cc3c2cc1C ; Cc1cc2c3cc4oc5ccccc5c4cc3c3ccccc3c2c(C)c1-c1cc(-c2ccccc2)nc(-c2ccccc2)n1]

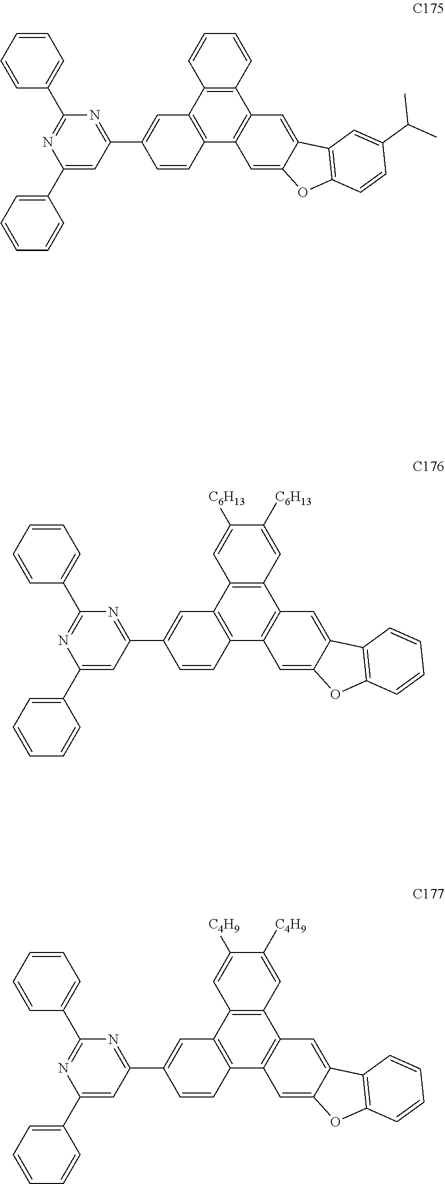

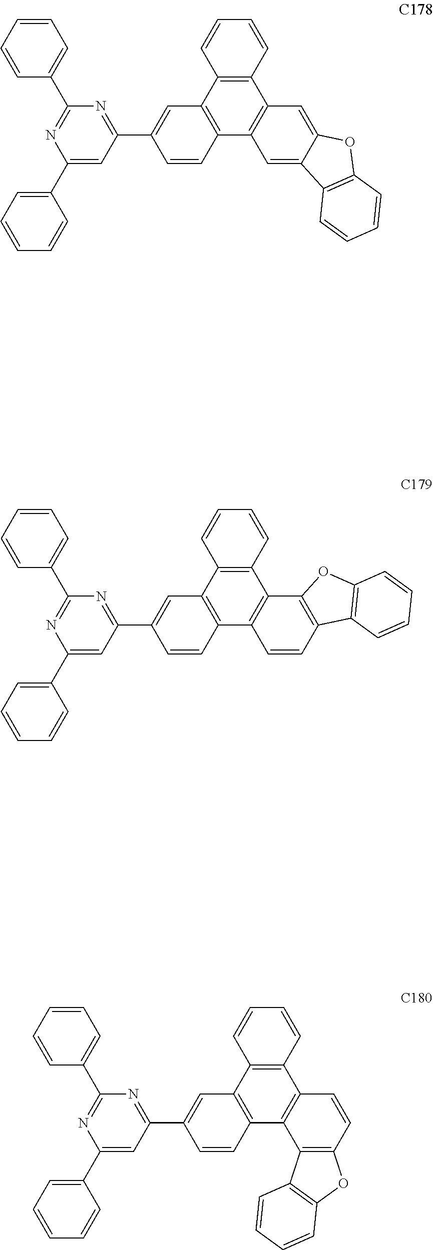 [SMILES: c1ccc(-c2cc(-c3ccc4c(c3)c3ccccc3c3cc5oc6ccccc6c5cc43)nc(-c3ccccc3)n2)cc1 ; c1ccc(-c2cc(-c3ccc4c(c3)c3ccccc3c3c4ccc4c5ccccc5oc43)nc(-c3ccccc3)n2)cc1 ; c1ccc(-c2cc(-c3ccc4c(c3)c3ccccc3c3ccc5oc6ccccc6c5c34)nc(-c3ccccc3)n2)cc1]

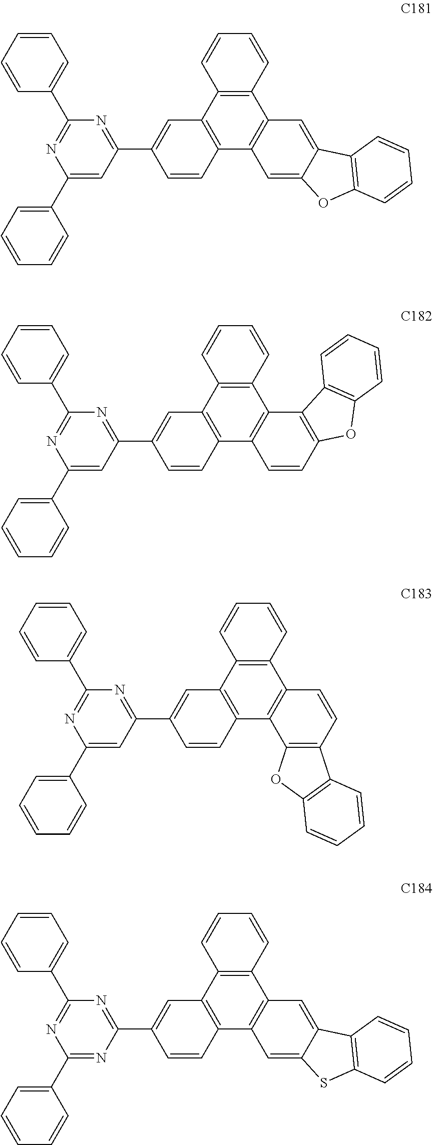 [SMILES: c1ccc(-c2cc(-c3ccc4c(c3)c3ccccc3c3cc5c(cc43)oc3ccccc35)nc(-c3ccccc3)n2)cc1 ; c1ccc(-c2cc(-c3ccc4c(c3)c3ccccc3c3c4ccc4oc5ccccc5c43)nc(-c3ccccc3)n2)cc1 ; c1ccc(-c2cc(-c3ccc4c(c3)c3ccccc3c3ccc5c6ccccc6oc5c34)nc(-c3ccccc3)n2)cc1 ; c1ccc(-c2nc(-c3ccccc3)nc(-c3ccc4c(c3)c3ccccc3c3cc5c(cc43)sc3ccccc35)n2)cc1]

In many cases, an organic light emitting device may be regarded as composed of a receptor and a donor organic compound. The receptor the organic light emitting device, the compounds, structure and the order with each other of the organic thin film layers, the thickness of each of the organic thin film layers, and the order of collocation with each other, have the characteristics of richness and diversity. By the richness and diversity, the receptor may have different structures, activities and functions.

Moreover, in an organic light emitting device, the organic compounds of the organic thin film layers are highly selective. If an organic compound is comprised by different organic thin film layers, the organic light emitting device may have completely different performance. Furthermore, it should be considered that the mechanism of interaction between he organic compounds of the organic thin film layers has specificity and complexity. If only the name of an organic thin film layer is disclosed, or only a simple text prompt is disclosed, it will not be enabled to solve key technical problems. At best, it can only provide directions for subsequent development.

According to one or more aspects of the present disclosure, a blue organic light emitting diode comprises a first electrode, a second electrode, and an organic thin film layer between the first electrode and the second electrode. The organic thin film layer is selected from the group consisting of a light emitting layer, a hole blocking layer, an electron transport layer, and combinations thereof, wherein the first light emitting layer comprises a first host and a first guest, and wherein the organic thin film layer comprises the organic compound of the present disclosure.

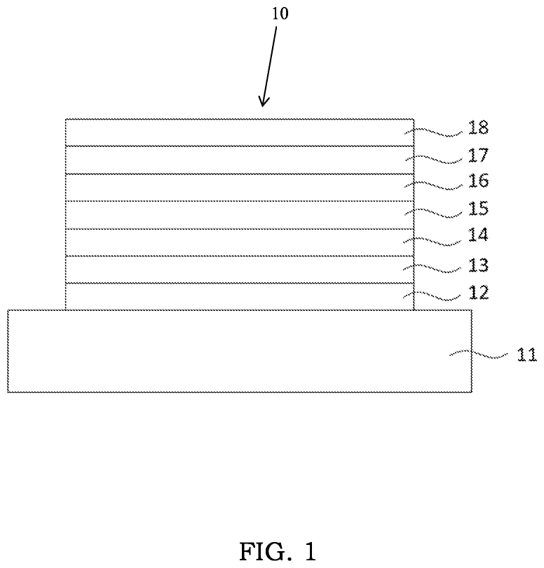

FIG. 1 shows a schematic cross-sectional view of a first embodiment of a blue OLED of the present disclosure. Referring to FIG. 1, a blue OLED 10 comprises a substrate 11, a first electrode 12, a hole injection layer 13, a hole transport layer 14, an electron blocking layer 15, a light emitting layer 16, an electron transport layer 17 and a second electrode 18.

The first electrode 12 is on the substrate 11. The hole injection layer 13, the hole transport layer 14, and the electron blocking layer 15 are formed on the first electrode 12. The light emitting layer 16 is formed on the electron blocking layer 15.

The light emitting layer 16 comprises a blue dopant and a blue host. Each of the blue host and the blue dopant may comprise an organic compound of the present disclosure as a material. The blue dopant accounts for 1% to 5% of the volume of the light emitting layer 16. The blue host may be selected from the organic compounds of the present disclosure. The blue host accounts for 95% to 99% of the volume of the light emitting layer 16. The electron transport layer 17 is formed on the light emitting layer 16. The material of the electron transport layer 16 may also comprise an organic compound selected from the organic compounds of the present disclosure. The electron transport layer 17 may comprise the organic compound of the present disclosure and comprise a co-evaporated material (LiQ). The organic compound of the present disclosure accounts for 50%-60% of the volume of the electron transport layer 17. The co-evaporated material accounts for 40%-50% of the volume of the electron transport layer 17.

The first electrode 12 is selected from the group consisting of indium tin oxide, indium zinc oxide, aluminum zinc oxide, and combinations thereof. The second electrode 18 may be selected from the group consisting of aluminum, magnesium, calcium, silver, and combinations thereof. The organic compound may be selected from the group consisting of the following compounds:

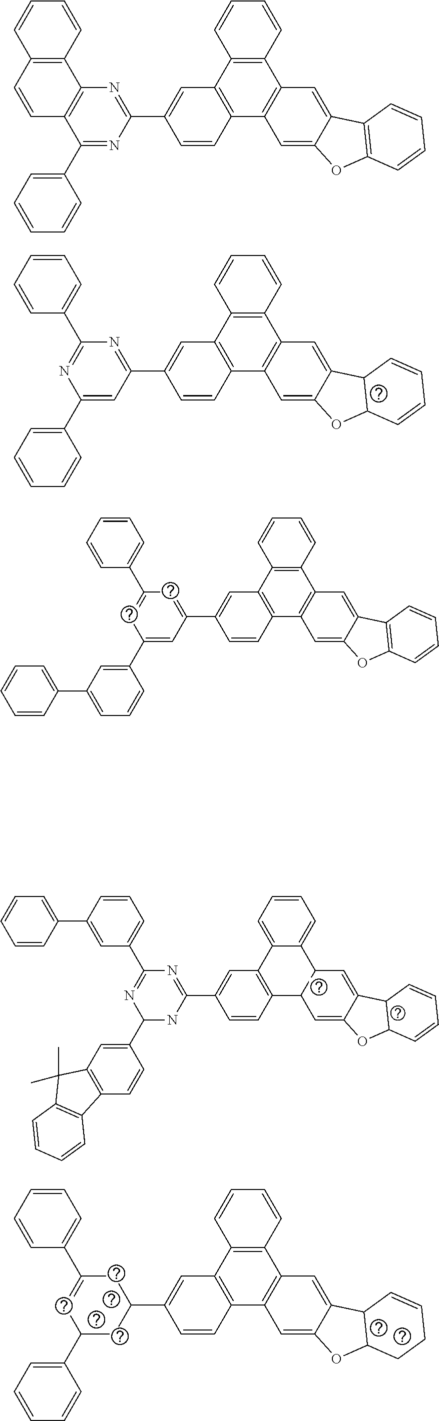

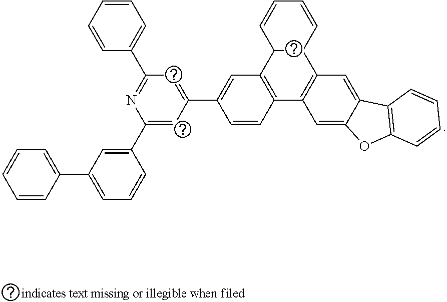 [SMILES: C1=CC2c3cc(-c4cc(-c5cccc(-c6ccccc6)c5)nc(-c5ccccc5)n4)ccc3-c3cc4oc5ccccc5c4cc3C2C=C1]

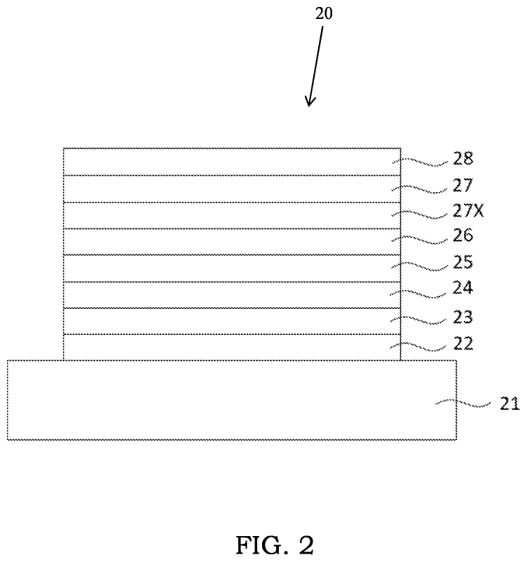

FIG. 2 shows a schematic cross-sectional view of a second embodiment of a blue OLED of the present disclosure. Referring to FIG. 2, a blue OLED 20 comprises a substrate 21, a first electrode 22, a hole injection layer 23, a hole transport layer 24, an electron blocking layer 25, a light emitting layer 26, a hole blocking layer 27X, an electron transport layer 27 and a second electrode 28.

The first electrode 22 is on the substrate 21. The hole injection layer 23, the hole transport layer 24, the electron blocking layer 25 and the light emitting layer 26 are formed on the first electrode 22. A light emitting layer 26 is formed on the electron blocking layer 25.

The light emitting layer 26 comprises a blue dopant and a blue host. Each of the blue host and the blue dopant may comprise an organic compound of the present disclosure as a material. The blue dopant accounts for 1% to 5% of the volume of the light emitting layer 26. The blue host may be selected from the organic compounds of the present disclosure. The blue host accounts for 95% to 99% of the volume of the light emitting layer 26.

The hole blocking layer 27X is formed on the light emitting layer 26. The hole blocking layer 27X is formed on the electron transport layer 27. The electron transport layer 27 may comprise the organic compound of the present disclosure and comprise a co-evaporated material (e.g., LiQ). The organic compound of the present disclosure accounts for 50%-60% of the volume of the electron transport layer 27. The co-evaporated material accounts for 40%-50% of the volume of the electron transport layer 27. The hole blocking layer 27X may also comprise the organic compound of the present disclosure. The second electrode 28 is formed on the electron transport layer 27. At least one of the first electrode 22 and the second electrode 28 may be transparent or semi-transparent for the emitted light to penetrate.

Preferably, the blue OLED may comprise a hole blocking layer between the light emitting layer and the electron transport layer. The hole blocking layer or the electron transport layer may comprise one of the following compounds:

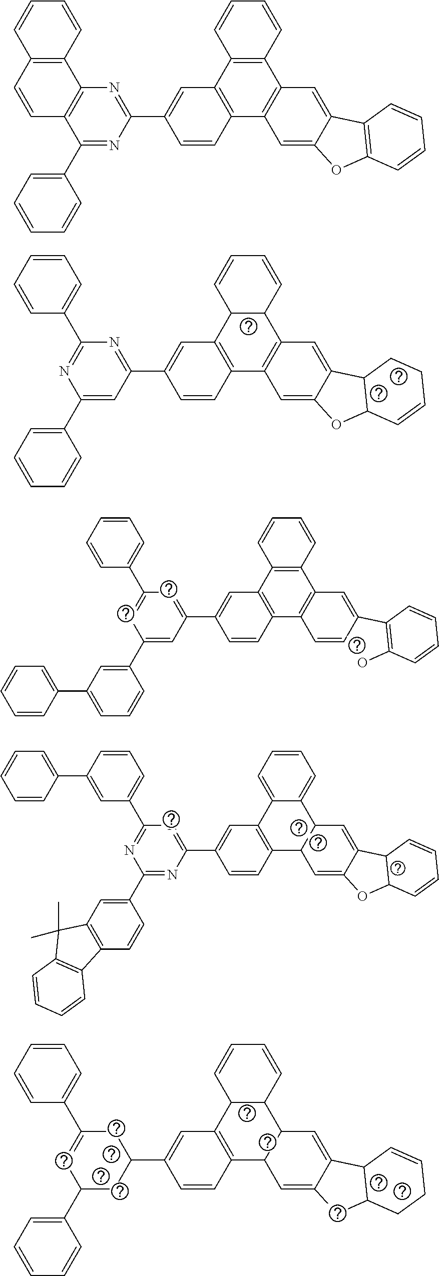

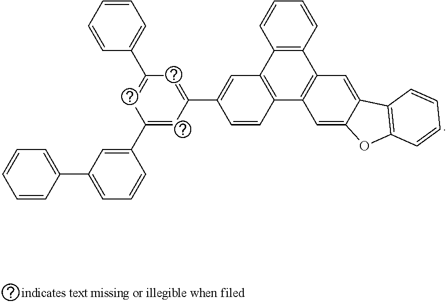 [SMILES: c1ccc(-c2cccc(-c3cc(-c4ccc5c(c4)c4ccccc4c4cc6c(cc54)oc4ccccc46)nc(-c4ccccc4)n3)c2)cc1]

More preferable, the hole blocking layer or the electron transport layer may comprise the same compound.

Referring to Table 1 and Table 2, compared to prior art, the inventive device examples of the present invention disclose blue OLEDs having higher device efficiencies or having higher lifetime

TABLE 1

| | Volt- | Current | Color | |
|---|---|---|---|---|
| | age | efficiency | coordinates | LT₉₅ |
| Examples | (V) | (cd/A) | (x, y) | (hrs) |
| Comparative Example 1 | 4.6 | 8.6 | (0.141, 0.173) | 37 |
| Inventive Device Example 1 | 4.6 | 8.7 | (0.141, 0.172) | 69 |
| Inventive Device Example 2 | 4.6 | 8.8 | (0.141, 0.172) | 57 |
| Inventive Device Example 3 | 4.5 | 9.1 | (0.140, 0.172) | 89 |
| Inventive Device Example 4 | 4.6 | 8.7 | (0.141, 0.172) | 79 |
| Inventive Device Example 5 | 4.5 | 9.1 | (0.141, 0.171) | 103 |
| Inventive Device Example 6 | 4.5 | 9.3 | (0.140, 0.170) | 135 |

TABLE 2

| | Volt- | Current | Color | |
|---|---|---|---|---|
| | age | efficiency | coordinates | LT₉₅ |
| Examples | (V) | (cd/A) | (x, y) | (hrs) |
| Comparative Example 2 | 4.7 | 8.5 | (0.140, 0.170) | 47 |
| Inventive Device Example 7 | 4.7 | 8.6 | (0.139, 0.170) | 84 |
| Inventive Device Example 8 | 4.6 | 8.8 | (0.139, 0.169) | 137 |
| Inventive Device Example 9 | 4.6 | 8.9 | (0.138, 0.168) | 160 |
| Inventive Device Example 10 | 4.7 | 8.6 | (0.139, 0.170) | 121 |
| Inventive Device Example 11 | 4.7 | 8.7 | (0.139, 0.169) | 145 |
| Inventive Device Example 12 | 4.6 | 9.1 | (0.138, 0.167) | 178 |

Referring to Table 1 and Table 2, in each Inventive Device Example, the electron transport layer of the blue organic light-emitting device may comprise an organic compound of the present disclosure. In each Inventive Device Example, the hole blocking layer of the blue organic light-emitting device may also comprise an organic compound of the present disclosure. The organic compounds of the hole blocking layer and the electron transport layer may be the same or similar with each other.

Referring to Table 2, the organic compounds of the hole blocking layer and the electron transport layer may be, but not limited to (C181; C70), (C70; C70), (C22; C22), (C1; C22), (C22 ; C1) or (C1; C1). Accordingly, if the host material of the light emitting layer, and the material of the electron blocking layer, are formed of the same material or similar material, it is helpful for improved efficiency and/or for extended lifetime.

### PRODUCTION OF COMPARATIVE EXAMPLE 1

An ITO glass, having a thickness of about 1250 angstroms (Å) and a sheet resistance of 15 ohms/unit area, serves as an ITO glass substrate. Before loading the pre-patterned ITO glass substrate into a vacuum evaporation chamber, the surface of the ITO glass substrate is cleaned with a cleaning agent, is then baked, and is irradiate with ultraviolet ozone. The ITO glass substrate is transferred to the vacuum evaporation chamber, to form an ITO electrode as a first electrode on the ITO glass substrate. A plurality of compounds, as shown in paragraph [0044], are evaporated and respectively formed organic layers on the first electrode. Finally, an upper metal is formed to serves as a second electrode on the organic layers.

By a heated vapor deposition source at a vacuum of about 10⁻⁶ Torr, the compounds, shown in paragraph [0044], are sequentially vapor-deposited to form a hole injection layer (HIL) with a thickness of about 200 angstroms (Å), a hole transport layer (HTL) with a thickness of about 1850 angstroms (Å), and an electron blocking layer (EBL) with a thickness of 50 angstroms (Å). A light emitting layer is subsequently formed with a thickness of about 300 angstroms (Å) on the electron blocking layer (EBL). The light emitting layer comprises a blue dopant and a blue host.

The blue host accounts for 95% of the volume of the light emitting layer. The blue dopant accounts for 5% of the volume of the light emitting layer. Compounds ETL and LiQ is then co-evaporated at a ratio of 1:0.8 to form an electron transport layer (ETL having LiQ) having a thickness of about 300 angstroms (Å) on the on the light emitting layer. Finally, Al is plated with a thickness of 1000 about angstroms (Å) as a second electrode. Subsequently, the OLED is transferred from the evaporation chamber to the drying box, and is packaged with a UV curable glue and a glass cover plate containing a moisture absorbent. The OLED emits blue light with a light emitting area of 0.25 square centimeters. In Table 1, to characterize the OLED, the voltage, current efficiency, and luminous efficiency are measured at 1000 nits by a constant current source (KEITHLEY 2400) and a photometer (PHOTO RESEARCH SpectraScan PR 650) at room temperature. The lifetime LT95 at a constant brightness of about 4000 nits is also measured and listed in Table 1. The LT₉₅ value is defined as the time required for the brightness level to drop to 95% relative to the initial brightness, which is used as a measure of the lifetime of an OLED.

Detailed structures, preparation and applied examples of the organic compounds of the present disclosure will be clarified by exemplary embodiments below, but the present disclosure is not limited thereto. Production of Inventive Device Examples 1-12 show the applied examples of the organic compounds of the present disclosure. Among them, the Inventive Device Examples 1-12 are disclosed to illustrate the production and activities of a variety of different blue OLEDs, and their test reports, as shown in Table 1 and Table 2. In the reports, using CIE coordinates well known in the art, to measure the luminous colour of the devices. Note that it is meaningful to compare the data of examples in the same conditions. If the conditions, such as device colours, comparative examples, thickness of the organic thin film layers, organic compounds or combination ratio, are not the same, will cause the data to change.

### PRODUCTION OF INVENTIVE DEVICE EXAMPLE 1

Similarly, referring to the production parameters of Comparative Example 1, to produce Inventive Device Example 1, except that the material of ETL in Comparative Example 1 is replaced with an organic compound C154. The other conditions are the same as Comparative Example 1.

### PRODUCTION OF INVENTIVE DEVICE EXAMPLE 2

Referring to the production parameters of Comparative Example 1, to produce Inventive Device Example 2, except that the material of ETL in Comparative Example 1 is replaced with an organic compound C181. The other conditions are the same as Comparative Example 1.

### PRODUCTION OF INVENTIVE DEVICE EXAMPLE 3

Referring to the production parameters of Comparative Example 1, to produce Inventive Device Example 3, except that the material of ETL in Comparative Example 1 is replaced with an organic compound C70. The other conditions are the same as Comparative Example 1.

### PRODUCTION OF INVENTIVE DEVICE EXAMPLE 4

Referring to the production parameters of Comparative Example 4, to produce Inventive Device Example 4, except that the material of ETL in Comparative Example 1 is replaced with an organic compound C106. The other conditions are the same as Comparative Example 1.

### PRODUCTION OF INVENTIVE DEVICE EXAMPLE 5

Referring to the production parameters of Inventive Device Example 2, to produce Inventive Device Example 5, except that t the material of ETL in Comparative Example 1 is replaced with an organic compound C22. The other conditions are the same as Inventive Device Example 1.

### PRODUCTION OF INVENTIVE DEVICE EXAMPLE 6

Referring to the production parameters of Inventive Device Example 5, to produce Inventive Device Example 6, except that the material of ETL in Comparative Example 1 is replaced with an organic compound C1. The other conditions are the same as Inventive Device Example 1.

When non-obviousness of the present disclosure is evaluated, the technical solution of the invention cannot be required to produce an advantageous technical effect in any situation and in all aspects. Such requirement does not comply with non-obviousness-related provisions of a patent law.

One person having ordinary skill in the art of the present application, in actual use, may select a material of a compound to take advantage of one kind of luminescent data (for example, to emit a specific color of light). In the same art of the present application, however, it is not always necessary for the present invention to take advantage of other kinds of luminescent data such as an operating voltage, current efficiency or lifetime of the device.

When the non-obviousness of the present disclosure is evaluated, it shall not be required to take advantage of all kinds of luminescent data. As long as the present invention takes advantage of one kind of luminescent data, such as a lower operating voltage, a higher current efficiency or a longer lifetime, the device of the present disclosure shall be regarded as producing an advantageous luminescent effect. It shall not be required to have a general improvement of all kinds of luminescent data of the compound in any case. Moreover, the present disclosure shall be considered as a whole. The technical effect brought by the whole technical solution should not be negated, even if some luminescent data of the compound are not good, or one luminescent data is not good for some kinds of color of light or for the application of some kinds of host.

A compound of the present application, as a material, shall not be required to improve all kinds of luminesce data, for all kinds of color of light, in the case of application of all kinds of host. As long as one kind of luminesce data, such as a current efficiency or lifetime of a specific color of light, is improved in the case of a specific host, the present invention shall be regarded as producing an advantageous technical effect. The advantageous technical effect is non-obvious enough to be a prominent substantive feature, so that the corresponding technical solution of the present disclosure involves an inventive step.

As shown in Table 1, the LT₉₅ of a blue OLED of Comparative Example 1 is about 37 hours, at a voltage of 4.6V and a blue color coordinate (0.141, 0.173). By replacing the ETL with an organic compound C154, LT₉₅ of the blue OLED of Inventive Device Example 1 is extended from about 37 hours to about 69 hours. Similar extension may also be shown in Inventive Device Examples 3, 5 and 6. In Inventive Device Example 3, LT₉₅ of the blue OLED is extended to about 89 hours, at a voltage of 4.5V and a blue color coordinate (0.140, 0.172). In Inventive Device Example 5, LT₉₅ of the blue OLED is extended to about 103 hours, at a voltage of 9.1V and a blue color coordinate (0.141, 0.172). In Inventive Device Example 6, LT₉₅ of the blue OLED is extended to about 135 hours, at a voltage of 4.5V and a blue color coordinate (0.140, 0.170). Compared to Comparative Example 1, the improvement of Inventive Device Example 6 is more significant, especially the improvement in life (LT₉₅).

### PRODUCTION OF COMPARATIVE EXAMPLE 2

Similarly, referring to the production parameters of Comparative Example 1, to produce Inventive Device Example 1, except that a layer is formed with a thickness of about 50 angstroms (Å) between the light emitting layer and the electron transport layer, as a hole blocking layer (HBL), and that the thickness of electron transport layer is changed to about 250 angstroms (Å). The ETL:LiQ ratio of the electron transport layer is about 1:0.8 after the HBL is formed. The other conditions are the same as Comparative Example 1.

### PRODUCTION OF INVENTIVE DEVICE EXAMPLE 7

Similarly, referring to the production parameters of Comparative Example 2, to produce Inventive Device Example 7, except that changing the 50 angstroms (Å) HBL in Comparative Example 2 to 50 angstroms (Å) compound C181 as the hole blocking layer, and that the ETL parameter is changed to C70:LiQ=1:0.8. The thickness of the ETL is to about 250 angstroms (Å). The other conditions are the same as Comparative Example 2.

### PRODUCTION OF INVENTIVE DEVICE EXAMPLE 8

Similarly, referring to the production parameters of Comparative Example 2, to produce Inventive Device Example 8, except that changing the 50 angstroms (Å) HBL in Comparative Example 2 to 50 angstroms (Å) compound C70 as the hole blocking layer, and that the ETL parameter is changed to C70:LiQ=1:0.8. The thickness of the ETL is to about 250 angstroms (Å). The other conditions are the same as Comparative Example 2.

### PRODUCTION OF INVENTIVE DEVICE EXAMPLE 9

Similarly, referring to the production parameters of Comparative Example 2, to produce Inventive Device Example 9, except that changing the 50 angstroms (Å) HBL in Comparative Example 2 to 50 angstroms (Å) compound C22 as the hole blocking layer, and that the ETL parameter is changed to C22:LiQ=1:0.8. The thickness of the ETL is to about 250 angstroms (Å). The other conditions are the same as Comparative Example 2.

### PRODUCTION OF INVENTIVE DEVICE EXAMPLE 10

Similarly, referring to the production parameters of Comparative Example 2, to produce Inventive Device Example 10, except that changing the 50 angstroms (Å) HBL in Comparative Example 2 to 50 angstroms (Å) compound C1 as the hole blocking layer, and that the ETL parameter is changed to C22:LiQ=1:0.8. The thickness of the ETL is to about 250 angstroms (Å). The other conditions are the same as Comparative Example 2.

### PRODUCTION OF INVENTIVE DEVICE EXAMPLE 11

Similarly, referring to the production parameters of Comparative Example 2, to produce Inventive Device Example 11, except that changing the 50 angstroms (ÅA) HBL in Comparative Example 2 to 50 angstroms (Å) compound C22 as the hole blocking layer, and that the ETL parameter is changed to C1:LiQ=1:0.8. The thickness of the ETL is to about 250 angstroms (Å). The other conditions are the same as Comparative Example 2.

### PRODUCTION OF INVENTIVE DEVICE EXAMPLE 12

Similarly, referring to the production parameters of Comparative Example 2, to produce Inventive Device Example 12, except that changing the 50 angstroms (Å) HBL in Comparative Example 2 to 50 angstroms (Å) compound C1 as the hole blocking layer, and that the ETL parameter is changed to C1:LiQ=1:0.8. The thickness of the ETL is to about 250 angstroms (Å). The other conditions are the same as Comparative Example 2.

As shown in Table 2, the LT₉₅ of a blue OLED of Comparative Example 2 is about 47 hours, at a voltage of 4.7V and a blue color coordinate (0.140, 0.170). After the ETL is replaced with an organic compound C70, Inventive Device Example 7 has similar performance in operating voltage, current efficiency, and luminous color coordinates. However, LT₉₅ of the blue OLED of Inventive Device Example 1 is extended from about 47 hours to about 84 hours. Accordingly, compound C181 is a preferable organic compound for a hole blocking layer of Inventive Device Example 7. Compound C70 is a preferable organic compound for an electron transport layer of Inventive Device Example 7.

In Inventive Device Example 8, the hole blocking layer and the electron transport layer both comprise the organic compound C70. The blue OLED has LT95 of the blue OLED is extended to about 137 hours, at a voltage of 4.6V and a blue color coordinate (0.139, 0.169). Accordingly, compound C70 is a preferable organic compound for a hole blocking layer of Inventive Device Example 8. Compound C70 is also a preferable organic compound for an electron transport layer of Inventive Device Example 8. Similar improvement may also be shown in Inventive Device Examples 9-12. Compared to Comparative Example 2, the improvement of Inventive Device Examples 9-12 is significant, especially the improvement in life (LT₉₅).

In some cases, the blue OLED is a lighting panel or a backlight panel.

### PREPARATION EXAMPLE 1 (C184)

### Synthesis of 1-bromo-2-iodo-4-methoxybenzene

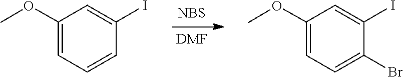

A mixture of 40 g (171 mmol) of 1-iodo-3-methoxybenzene, 32 g (179 mmol) of N-bromosuccinimide, and 600 ml of DMF was degassed and placed under nitrogen, and then heated at 80° C. for 12 hrs. After the reaction finished, the mixture was allowed to cool to room temperature. Subsequently, the solvent was removed under reduced pressure, and the crude product was purified by column chromatography, yielding 45 g of 1-bromo-2-iodo-4-methoxybenzene as yellow oil (84.1%). ¹H NMR (CDCl₃, 400 MHz): chemical shift (ppm) 7.43 (dd, 1H), 7.35 (dd, 1H), 6.73 (dd, 1H), 3.74 (s, 3H).

### Synthesis of 2-bromo-5-methoxy-1,1′-biphenyl

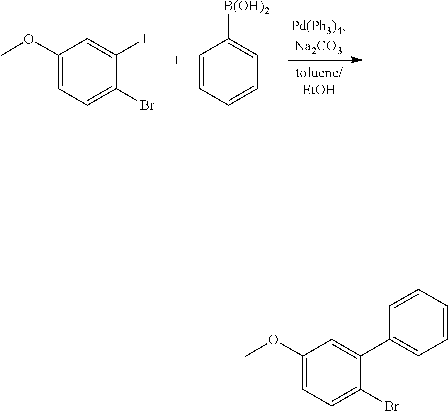

A mixture of 40 g (127.8 mmol) of 1-bromo-2-iodo-4-methoxybenzene, 15.6 g (127.8 mmol) of phenylboronic acid, 2.95 g (2.56 mmol) of Pd(Ph₃)₄, 155 ml of 2M Na₂CO₃, 100 ml of EtOH and 300 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 30 g of 2-bromo-5-methoxy-1,1′-biphenyl as colorless liquid (89.2%). ¹H NMR (CDCl₃, 400 MHz): chemical shift (ppm) 7.55 (d, 1H), 7.46-7.38 (m, 5H), 6.89 (d, 1H), 6.79 (dd, 1H), 3.81 (s, 3H).

### Synthesis of (5-methoxy-[1,1′-biphenyl]-2-yl) boronic acid

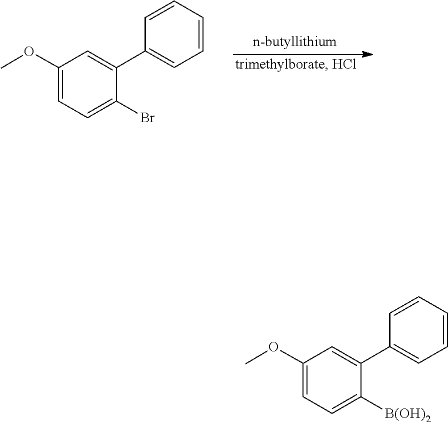 [SMILES: COc1ccc(Br)c(-c2ccccc2)c1 ; "n-butyllithium" ; "trimethylborate, HCl" ; COc1ccc(B(O)O)c(-c2ccccc2)c1]

The compound 2-bromo-5-methoxy-1,1′-biphenyl (30 g, 114 mmol) was mixed with 600 ml of dry THF. To the mixture, 54.7 ml of N-butyllithium (137 mmol) was added at −60° C. and the mixture was stirred for 1 hrs. After the reaction finished, 17.8 g (171 mmol) of trimethyl borate was added and the mixture was stirred overnight. 228 ml (228 mmole) of 1M HCl was added and the mixture was stirred for 1 hrs. The mixture was extracted with ethyl acetate/H₂O, and the organic layer was removed under reduced pressure. The crude product was washed by hexane, yielding 19.5 g of (5-methoxy-[1,1′-biphenyl]-2-yl) boronic acid as white solid (75%).

### Synthesis of 3-(5-methoxy-[1,1′-biphenyl]-2-yl) dibenzo [b,d] -thiophene

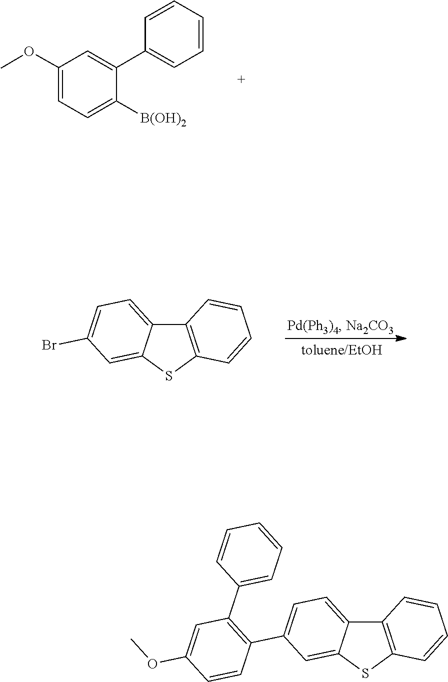

A mixture of 20 g (87.7 mmol) of (5-methoxy-[1,1′-biphenyl]-2-yl)- boronic acid, 25.4 g (96.5 mmol) of 3-bromodibenzo[b,d]thiophene, 2.03 g (1.75 mmol) of Pd(Ph₃)₄, 87.7 ml of 2M Na₂CO₃,200 ml of EtOH and 400 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 23.1 g of 3-(5-methoxy-[1,1′-biphenyl]-2-yl)-dibenzo[b,d]thiophene as white solid (71.9%). ¹H NMR (CDCl₃, 400 MHz): chemical shift (ppm) 8.47 (d, 1H), 8.12-8.06 (m, 3H), 8.01 (d, 1H), 7.77-7.74 (m, 3H), 7.49-7.45 (m, 4H), 7.41-7.38 (m, 2H), 7.02 (d, 1H), 3.81 (s, 3H).

### Synthesis of 6-methoxybenzo[b]triphenyleno[2,3-d]thiophene

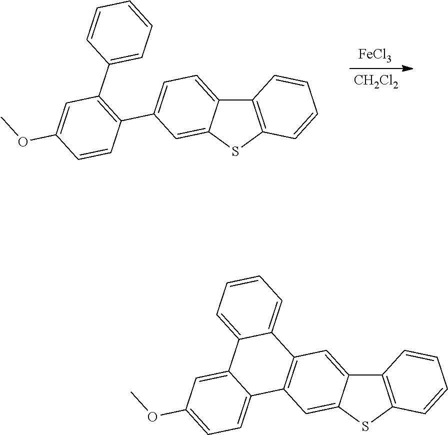

The compound 3-(5-methoxy-[1,1′-biphenyl]-2-yl)dibenzo[b,d]-thiophene (20 g, 54.6 mmol) was mixed with 700 ml of CH₂Cl₂. To the mixture, 88.5 g of FeCl₃(546 mmol) was added and the mixture was stirred for 1 hrs. After the reaction finished, the solvent was removed under reduced pressure, and the crude product was purified by column chromatography, yielding 8.5 g of 6-methoxybenzo[b]triphenyleno[2,3-d]-thiophene as white solid (42.7%). ¹H NMR (CDCl₃, 400 MHz): chemical shift (ppm) 8.91-8.89 (m, 2H), 8.81 (d, 1H), 8.49 (d, 1H), 8.14 (m, 2H), 7.99 (d, H), 7.89-7.85 (m, 2H), 7.62 (s, 1H), 7.54-7.51 (m, 2H), 7.36 (d, 1H), 3.82 (s, 3H).

### Synthesis of benzo[b]triphenyleno[2,3-d]thiophen-6-ol

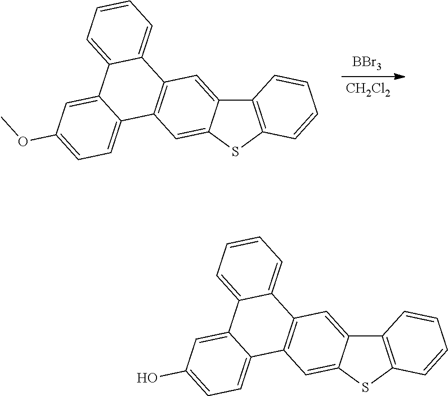

The compound 6-methoxybenzo[b]triphenyleno[2,3-d]-thiophene (10 g, 27.4 mmol) was mixed with 400 ml of CH₂Cl₂. To the mixture, 8.25 g of BBr₃(32.9 mmol) was added and the mixture was stirred overnight. After the reaction finished, the solvent was removed under reduced pressure, and the crude product was purified by column chromatography, yielding 8.8 g of benzo[b]triphenyleno[2,3-d]thiophen-6-ol as white solid (91.5%). ¹H NMR (CDCl₃, 400 MHz): chemical shift (ppm) 8.89-8.87 (m, 2H), 8.78 (d, 1H), 8.45 (d, 1H), 8.09 (m, 2H), 7.94 (d, H), 7.86-7.83 (m, 2H), 7.58 (s, 1H), 7.51-7.48(m, 2H), 7.31 (d, 1H), 5.41 (s, 1H).

### Synthesis of benzo[b]triphenyleno[2,3-d]thiophen-6-yl trifluoro-methanesulfonate

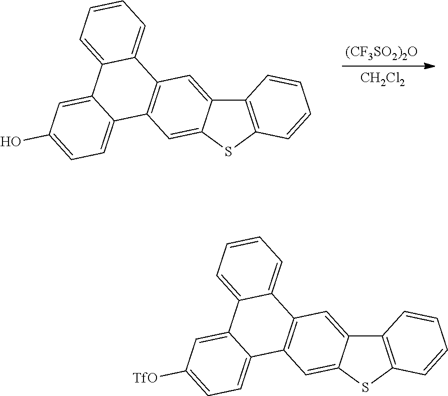 [SMILES: Oc1ccc2c(c1)c1ccccc1c1cc3c(cc21)sc1ccccc13 ; "(CF3SO2)2O" ; "CH2Cl2" ; O=S(=O)(Oc1ccc2c(c1)c1ccccc1c1cc3c(cc21)sc1ccccc13)C(F)(F)F]

The compound benzo[b]triphenyleno[2,3-d]thiophen-6-ol (10 g, 28.5 mmol) was mixed with 450 ml of CH₂Cl₂. To the mixture, 3.4 g of pyridine (42.8 mmol) was added and the mixture was stirred for 1 hrs. To the mixture, 13.7 g of (CF₃SO₂)₂O (48.5 mmol) was added and the mixture was stirred for 1 hrs. After the reaction finished, the solvent was removed under reduced pressure, and the crude product was purified by column chromatography, yielding 10.5 g of benzo[b]triphenyleno[2,3-d]thiophen-6-yl trifluoro-methanesulfonate as yellow solid (55.9%). ¹H NMR (CDCl₃, 400 MHz): chemical shift (ppm) 8.99-8.95 (m, 3H), 8.47 (d, 1H), 8.14-8.11 (m, 3H), 7.97 (d, H), 7.88-7.85 (m, 2H), 7.58 (s, 1H), 7.53-7.51 (m, 2H).

### Synthesis of 2-(benzo[b]triphenyleno[2,3-d]thiophen-6-yl)-4,4,5,5-tetramethyl-1,3,2-dioxaborolane

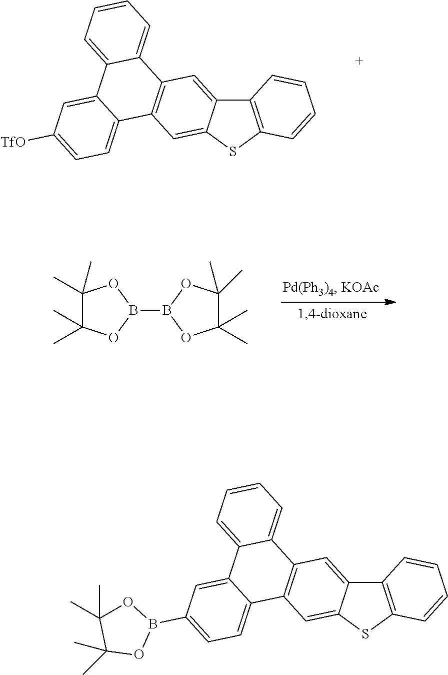

A mixture of 5 g (10.4 mmol) of benzo[b]triphenyleno[2,3-d]thiophen- 6-yl trifluoromethanesulfonate, 3.16 g (12.4 mmol) of bis(pinacolato)diboron, 0.48 g (0.4 mmol) of Pd(Ph₃)₄, 2.04 g (20.8 mmol) of potassium acetate, and 60 ml of 1,4-dioxane was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 3.1 g of 2-(benzo [b]triphenyleno [2,3-d]thiophen-6-yl) -4,4,5,5-tetramethyl-1,3 ,2-dioxa borolane as white solid (65%). ¹H NMR (CDCl₃, 400 MHz): chemical shift (ppm) 8.94-8.88 (m, 3H), 8.47 (d, 1H), 8.15-8.12 (m, 3H), 7.99 (d,1H), 7.87-7.84 (m, 3H), 7.54-7.52 (m, 2H), 1.27 (s, 12H).

### Synthesis of 2-(benzo[b]triphenyleno[2,3-d]thiophen-6-yl)-4,6-diphenyl-1,3,5-triazine (C184)

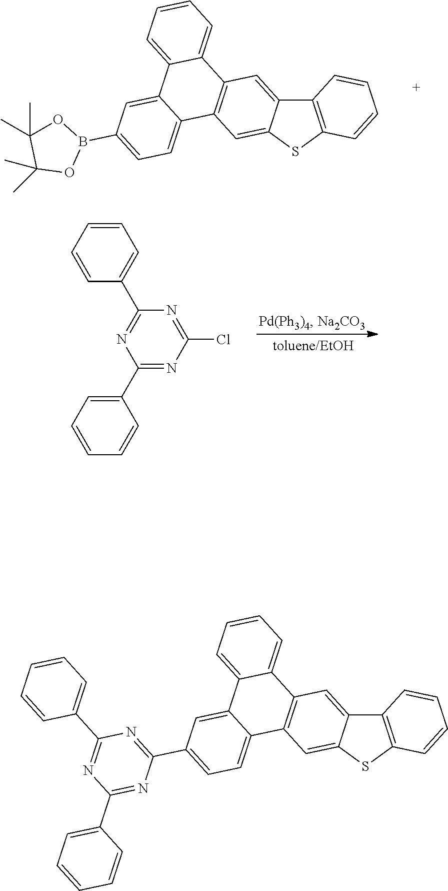

A mixture of 3 g (6.51 mmol) of 2-(benzo[b]triphenyleno[2,3-d]-thiophen-6-yl)-4,4,5,5-tetramethyl-1,3,2-diox aborolane, 1.92 g (7.17 mmol) of 92-chloro-4,6-diphenyl-1,3,5-triazine, 0.15 g (0.13 mmol) of Pd(Ph₃)₄, 6.5 ml of 2M Na₂CO₃, 20 ml of EtOH and 40 ml of toluene was degassed and placed under nitrogen, and then heated to reflux for 12 hrs. After the reaction finished, the mixture was allowed to cool to room temperature. Subsequently, the solvent was removed under reduced pressure, and the crude product was purified by column chromatography, yielding 2.5 g of 2-(benzo[b]triphenyleno[2,3-d]thiophen-6-yl)-4,6-diphenyl-1,3,5-triazine as yellow solid (68%). ¹H NMR (CDCl₃, 400 MHz): chemical shift (ppm) 8.99-8.93 (m, 3H), 8.46 (d, 1H), 8.33 (s, 1H), 8.29-8.24 (m, 4H), 8.13-8.09 (m, 3H), 7.97 (d, H), 7.88-7.82 (m, 2H), 7.53-7.46 (m, 6H), 7.44-7.41 (m, 2H).

### PREPARATION EXAMPLES 2-5

We have used the same synthesis methods to get a series of intermediates and the following compounds are synthesized analogously.

| Preparation Ex. | Intermediate III | Intermediate IV | Product |
|---|---|---|---|
| 2 | 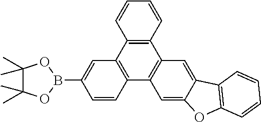 [SMILES: CC1(C)OB(c2ccc3c(c2)c2ccccc2c2cc4c(cc32)oc2ccccc24)OC1(C)C] | 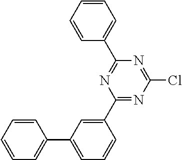 [SMILES: Clc1nc(-c2ccccc2)nc(-c2cccc(-c3ccccc3)c2)n1] | 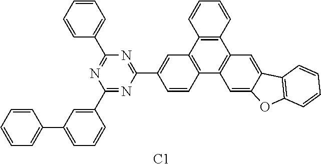 [SMILES: c1ccc(-c2cccc(-c3nc(-c4ccccc4)nc(-c4ccc5c(c4)c4ccccc4c4cc6c(cc54)oc4ccccc46)n3)c2)cc1 ; "C1"] |
| 3 | 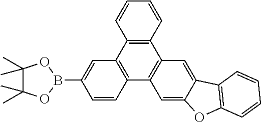 [SMILES: CC1(C)OB(c2ccc3c(c2)c2ccccc2c2cc4c(cc32)oc2ccccc24)OC1(C)C] | 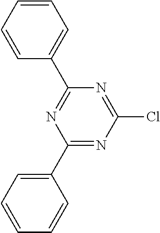 [SMILES: Clc1nc(-c2ccccc2)nc(-c2ccccc2)n1] | 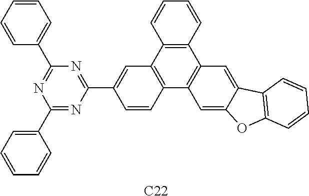 [SMILES: c1ccc(-c2nc(-c3ccccc3)nc(-c3ccc4c(c3)c3ccccc3c3cc5c(cc43)oc3ccccc35)n2)cc1 ; "C22"] |
| 4 | 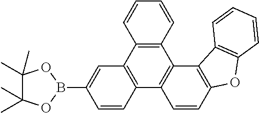 [SMILES: CC1(C)OB(c2ccc3c(c2)c2ccccc2c2c3ccc3oc4ccccc4c32)OC1(C)C] | 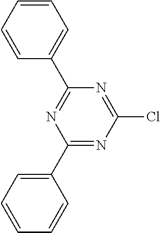 [SMILES: Clc1nc(-c2ccccc2)nc(-c2ccccc2)n1] | 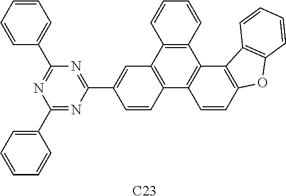 [SMILES: c1ccc(-c2nc(-c3ccccc3)nc(-c3ccc4c(c3)c3ccccc3c3c4ccc4oc5ccccc5c43)n2)cc1 ; "C23"] |

It is understood that the various embodiments described herein are by way of example only, and are not intended to limit the scope of the invention. For example, many of the materials and structures described herein may be substituted with other materials and structures without deviating from the spirit of the invention. The present invention as claimed may therefore include variations from the particular examples and preferred embodiments described herein, as will be apparent to one of skill in the art. It is understood that various theories as to why the invention works are not intended to be limiting.

What is claimed is:

1. An organic compound represented by the following formula (1): 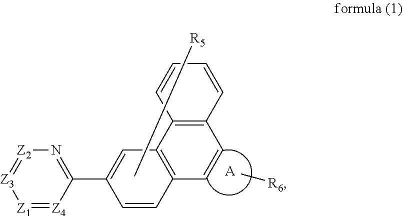
wherein A represents dibenzofuran, dibenzothiophene, dibenzoselenophene, 9,9-dialkylfluorene, 9,9-dialkyl-9H-9-silafluorenyl or carbazole;
where Z₁ is N or CR₁;
where Z₂ is N or CR₂;
where Z3 is N or CR₃;
where Z₄ is N or CR₄;
wherein at least one of Z₃ and Z₄ is N;
wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;
wherein each of R₅ and R₆ represents mono to a maximum possible number of substitutions, or no substitution; and
each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof.

2. The organic compound of claim 1, wherein one of the following is true:
R₁ and R₂ are not the same;
Z₁ is CR₁, and Z₂ is CR₂;
R₅ or R₆ is a mono substituent if R₁ and R₂ are both phenyl,
at least one of R₁ and R₂ is selected from the group consisting of 3-biphenyl, 2-biphenyl, 4-biphenyl, 9,9-dialkylfluorenyl, phenyl, methylphenyl, methyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, or 2-(4-pyridyl)phenyl, dibenzofuranyl, dibenzothienyl, dibenzoselenophenyl, and combinations thereof;
if one of R₁ and R₂ is 3-biphenyl, 2-biphenyl, or 4-biphenyl, the other is phenyl;
R₂ and R₃ are connected so that Z₂ and Z₃ together form a polycyclic aromatic group;
R₁ to R₄ are independently selected from the group consisting of H, phenyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3- (4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and
each of R₅ and R₆ is independently selected from the group consisting of no substitution, methyl, ethyl, propyl, butyl, hexyl, and combinations thereof.

3. The organic compound of claim 1, wherein one of the following is true:
Z₁ is CR₁, and Z₂ is CR₂;
at least one of R₁ and R₂ is selected from the group consisting of 3-biphenyl, 2-biphenyl, 4-biphenyl, 9,9-dialkylfluorenyl, dibenzofuranyl, dibenzothienyl, and combinations thereof if R₁ and R₂ are not the same;
R₅ or R₆ is selected from the group consisting of unsubstituted, methyl, ethyl, isopropyl, n-butyl, n-hexyl, and combinations thereof if R₁ and R₂ are both phenyl;
if one of R₁ and R₂ is 3-biphenyl, the other is phenyl;
Z₂ is CR₂, and Z₃ is CR₃;
R₂ and R₃ are connected so that Z₂ and Z₃ together form naphthalene or benzene;
Z₃ is N; and
Each of R₅ and R₆ is independently selected from the group consisting of methyl, ethyl, isopropyl, n-butyl, n-hexyl, and combinations thereof.

4. An organic compound, for extending lifetime or increasing efficiency of a blue organic light emitting device, the organic compound being represented by one of the following formulas (2) to (6): 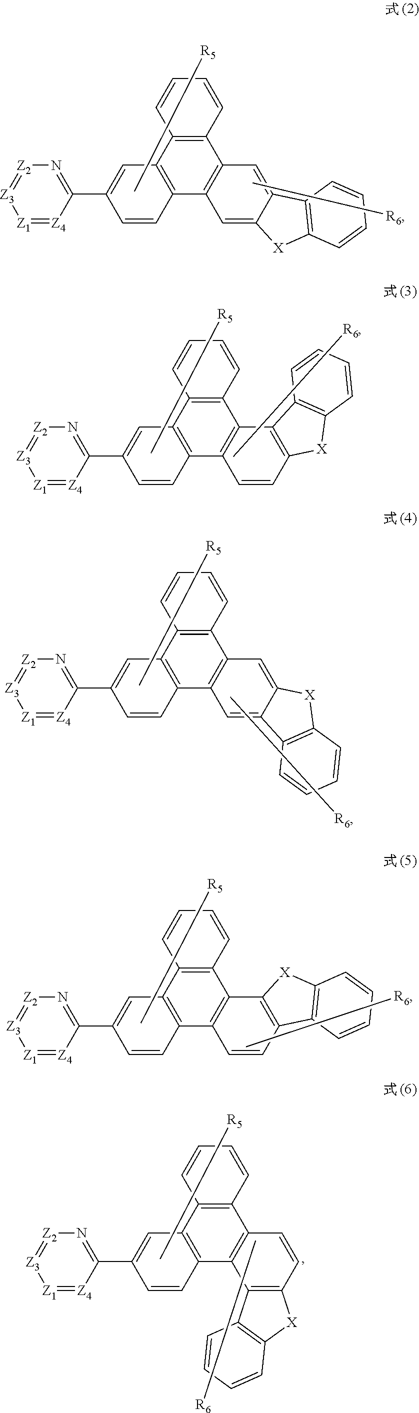
where Z₁ is N or CR₁;
where Z₂ is N or CR₂;
where Z₃ is N or CR₃;
where Z₄ is N or CR₄;
wherein at least one of Z₃ and Z₄ is N;
wherein R₁ to R₄ are independently selected from the group consisting of H, aryl, alkyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)benzene Group, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, 9,9-dialkyl-9H-9-silafluorenyl, and combinations thereof;
wherein each of R₅ and R₆ represents mono to a maximum possible number of substitutions, or no substitution;
each of R₅ and R₆ is independently selected from the group consisting of halogen, alkyl, phenyl, methylphenyl, pyridyl, 3-biphenyl, 2-biphenyl, 4-biphenyl, 4 -(3-pyridyl)phenyl, 4-(2-pyridyl)phenyl, 4-(4-pyridyl)phenyl, 3-(3-pyridyl)phenyl, 3-(2-pyridyl))Phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and
one of the following is true:
R₁ and R₂ are not the same;
Z₁ is CR₁, and Z₂ is CR₂;
R₅ or R₆ is a mono substituent if R₁ and R₂ are both phenyl,
at least one of R₁ and R₂ is selected from the group consisting of 3-biphenyl, 2-biphenyl, 4-biphenyl, 9,9-dialkylfluorenyl, phenyl, methylphenyl, methyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, or 2-(4-pyridyl)phenyl, dibenzofuranyl, dibenzothienyl, dibenzoselenophenyl, and combinations thereof;
if one of R₁ and R₂ is 3-biphenyl, 2-biphenyl, or 4-biphenyl, the other is phenyl;
R₂ and R₃ are connected so that Z₂ and Z₃ together form a polycyclic aromatic group;
R₁ to R₄ are independently selected from the group consisting of H, phenyl, alkylphenyl, pyridyl, 3-biphenyl, 2-biphenyl, 4-biphenyl, 4-(3-pyridyl)phenyl, 4-(2-pyridyl)phenyl, 4-(4-pyridyl)phenyl, 3-(3-pyridyl)phenyl, 3-(2-pyridyl)phenyl, 3-(4-pyridyl)phenyl, 2-(3-pyridyl)phenyl, 2-(2-pyridyl)phenyl, 2-(4-pyridyl)phenyl, 9,9-dialkylfluorenyl, dibenzofuranyl, dibenzothienyl, dibenzoselenophenyl, and combinations thereof; and
each of R₅ and R₆ is independently selected from the group consisting of no substitution, methyl, ethyl, propyl, butyl, hexyl, and combinations thereof.

5. The organic compound of claim 1, wherein the organic compound selected from the group consisting of the following compounds C1 to C184:

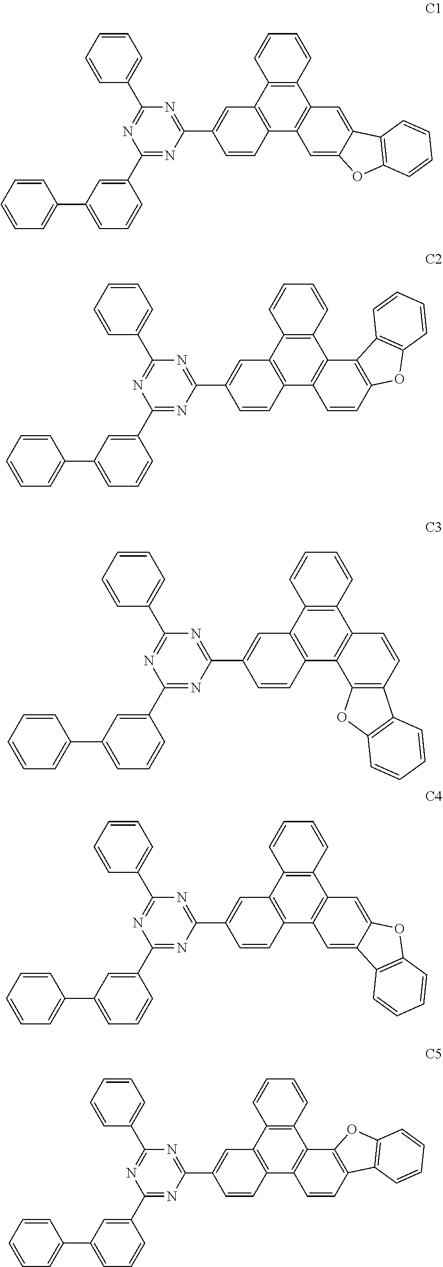 [SMILES: c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; c1ccc(-c2cccc(-c3nc(-c4ccccc4)nc(-c4ccc5c(c4)c4ccccc4c4ccc6c7ccccc7oc6c45)n3)c2)cc1 ; c1ccc(-c2cccc(-c3nc(-c4ccccc4)nc(-c4ccc5c(c4)c4ccccc4c4cc6oc7ccccc7c6cc54)n3)c2)cc1 ; c1ccc(-c2cccc(-c3nc(-c4ccccc4)nc(-c4ccc5c(c4)c4ccccc4c4c5ccc5c6ccccc6oc54)n3)c2)cc1]

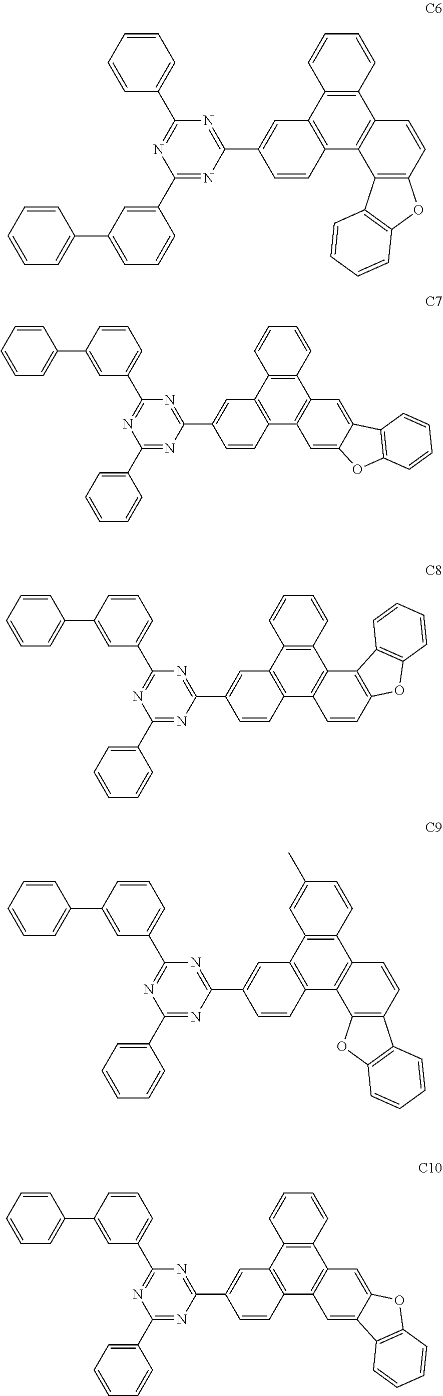 [SMILES: c1ccc(-c2cccc(-c3nc(-c4ccccc4)nc(-c4ccc5c(c4)c4ccccc4c4ccc6oc7ccccc7c6c45)n3)c2)cc1 ; c1ccc(-c2cccc(-c3nc(-c4ccccc4)nc(-c4ccc5c(c4)c4ccccc4c4cc6c(cc54)oc4ccccc46)n3)c2)cc1 ; c1ccc(-c2cccc(-c3nc(-c4ccccc4)nc(-c4ccc5c(c4)c4ccccc4c4c5ccc5oc6ccccc6c54)n3)c2)cc1 ; Cc1ccc2c(c1)c1cc(-c3nc(-c4ccccc4)nc(-c4cccc(-c5ccccc5)c4)n3)ccc1c1c2ccc2c3ccccc3oc21 ; c1ccc(-c2cccc(-c3nc(-c4ccccc4)nc(-c4ccc5c(c4)c4ccccc4c4cc6oc7ccccc7c6cc54)n3)c2)cc1]

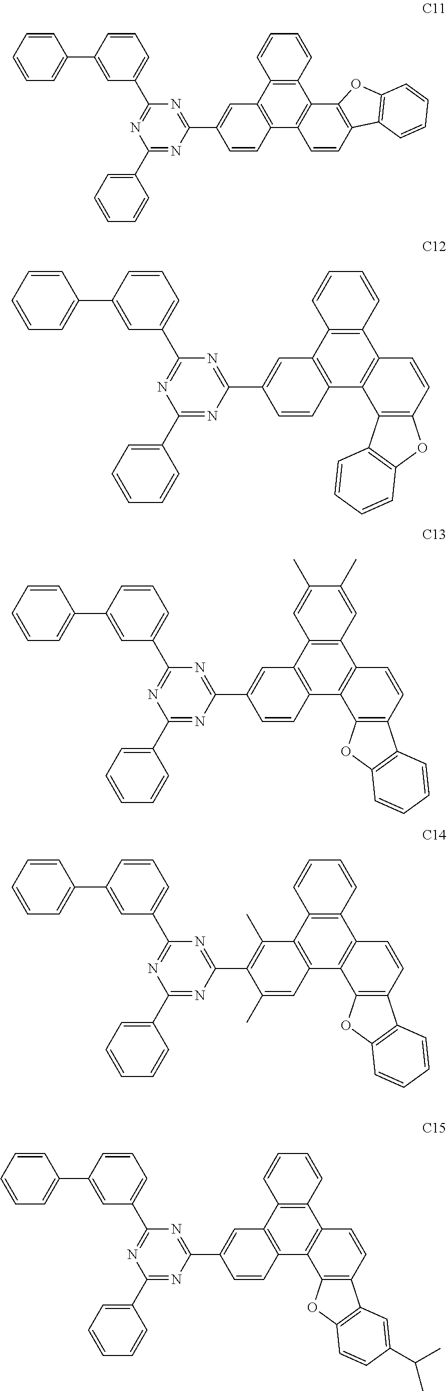 [SMILES: c1ccc(-c2cccc(-c3nc(-c4ccccc4)nc(-c4ccc5c(c4)c4ccccc4c4c5ccc5c6ccccc6oc54)n3)c2)cc1 ; c1ccc(-c2cccc(-c3nc(-c4ccccc4)nc(-c4ccc5c(c4)c4ccccc4c4ccc6oc7ccccc7c6c45)n3)c2)cc1 ; Cc1cc2c3cc(-c4nc(-c5ccccc5)nc(-c5cccc(-c6ccccc6)c5)n4)ccc3c3c(ccc4c5ccccc5oc43)c2cc1C ; Cc1cc2c(c(C)c1-c1nc(-c3ccccc3)nc(-c3cccc(-c4ccccc4)c3)n1)c1ccccc1c1ccc3c4ccccc4oc3c12 ; CC(C)c1ccc2oc3c(ccc4c5ccccc5c5cc(-c6nc(-c7ccccc7)nc(-c7cccc(-c8ccccc8)c7)n6)ccc5c43)c2c1]

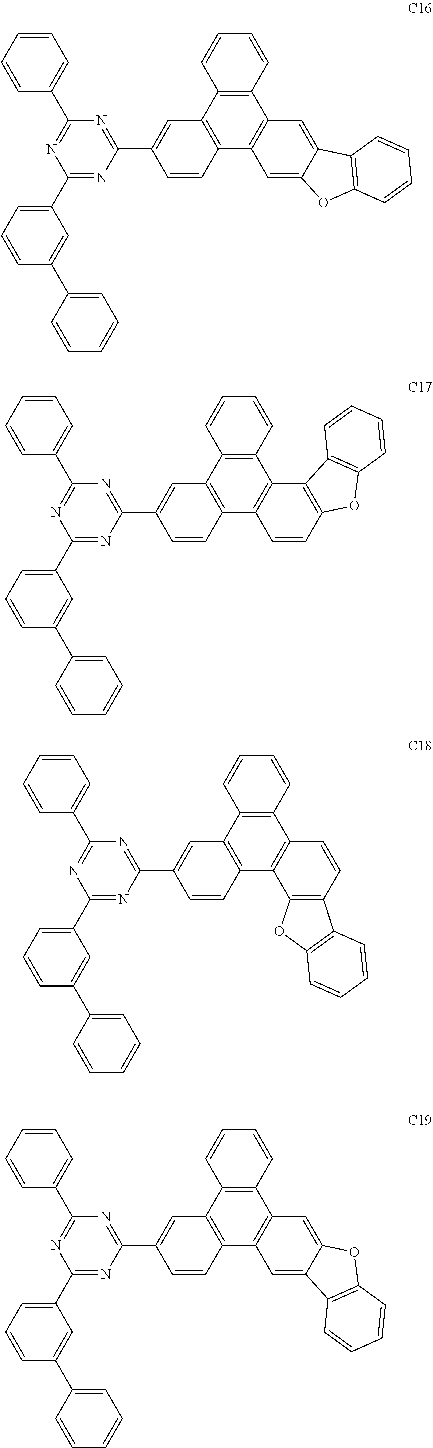

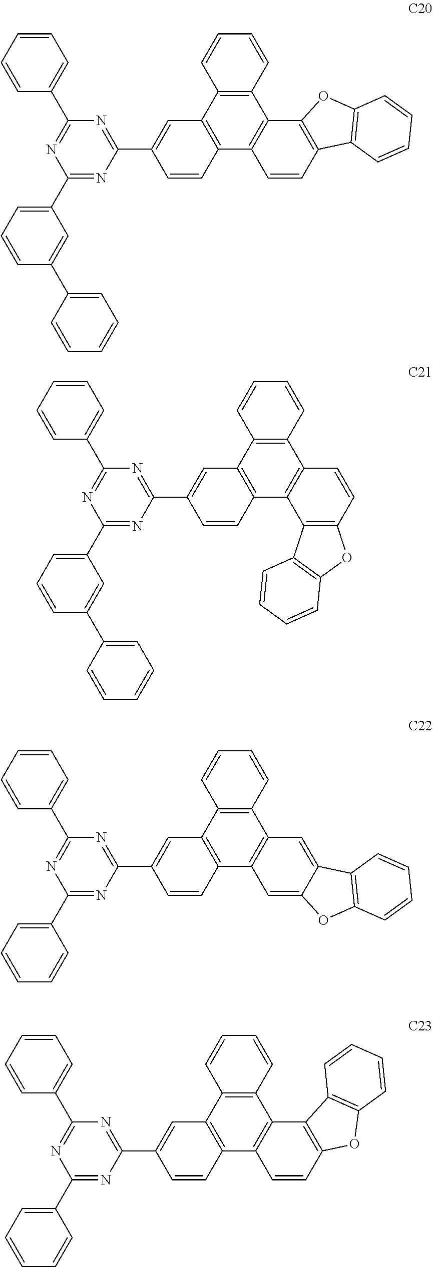

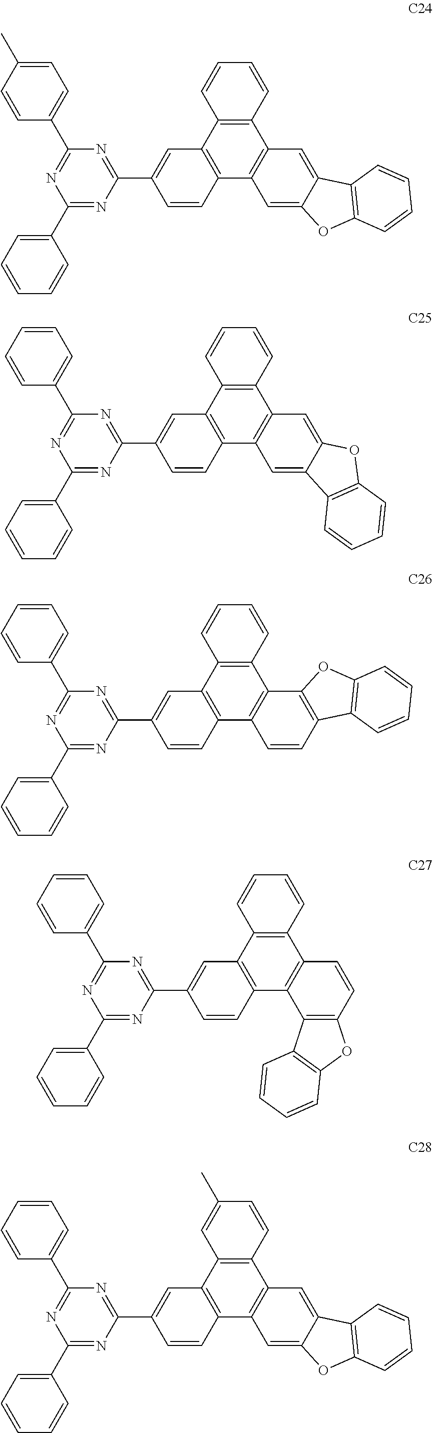

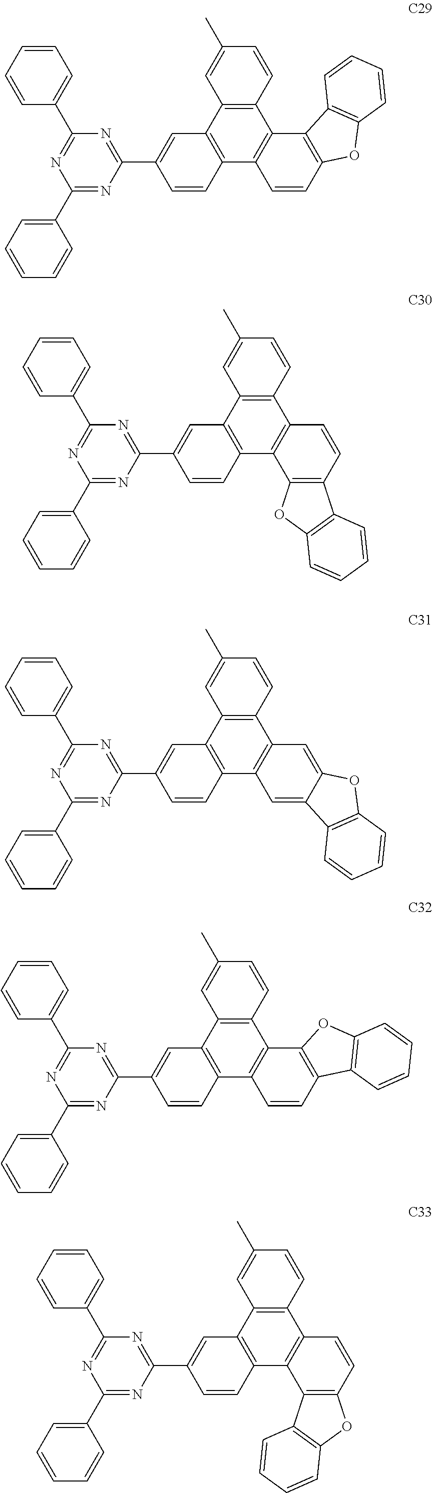 [SMILES: Cc1ccc2c(c1)c1cc(-c3nc(-c4ccccc4)nc(-c4ccccc4)n3)ccc1c1ccc3oc4ccccc4c3c12 ; Cc1ccc2c(c1)c1cc(-c3nc(-c4ccccc4)nc(-c4ccccc4)n3)ccc1c1c2ccc2c3ccccc3oc21 ; Cc1ccc2c(c1)c1cc(-c3nc(-c4ccccc4)nc(-c4ccccc4)n3)ccc1c1cc3c(cc21)oc1ccccc13 ; Cc1ccc2c(c1)c1cc(-c3nc(-c4ccccc4)nc(-c4ccccc4)n3)ccc1c1ccc3c4ccccc4oc3c12 ; Cc1ccc2c(c1)c1cc(-c3nc(-c4ccccc4)nc(-c4ccccc4)n3)ccc1c1c2ccc2oc3ccccc3c21]

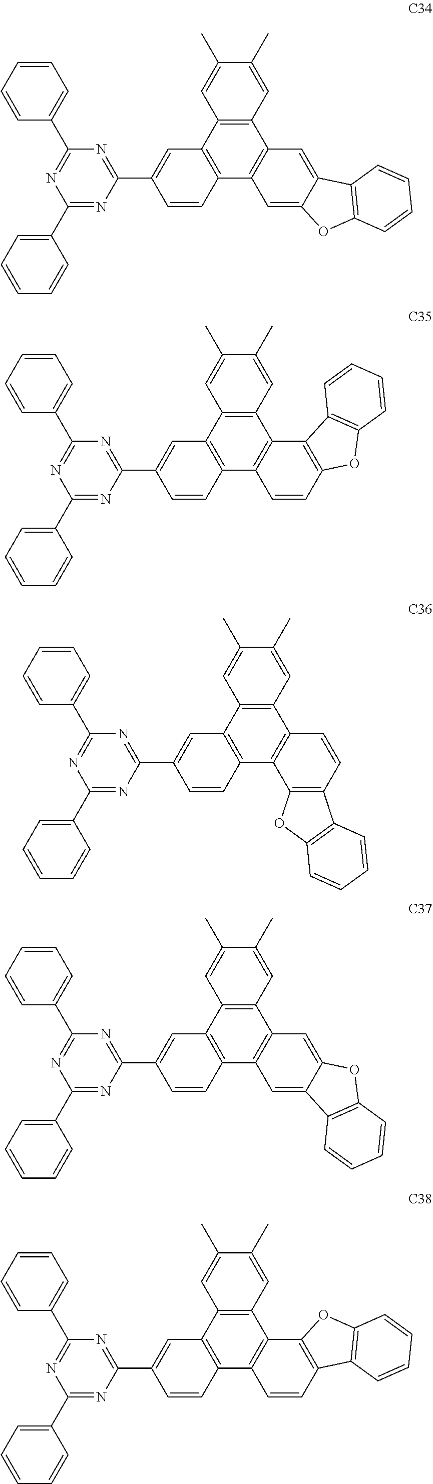 [SMILES: Cc1cc2c3cc(-c4nc(-c5ccccc5)nc(-c5ccccc5)n4)ccc3c3cc4oc5ccccc5c4cc3c2cc1C ; Cc1cc2c3cc(-c4nc(-c5ccccc5)nc(-c5ccccc5)n4)ccc3c3ccc4oc5ccccc5c4c3c2cc1C ; Cc1cc2c3cc(-c4nc(-c5ccccc5)nc(-c5ccccc5)n4)ccc3c3c(ccc4c5ccccc5oc43)c2cc1C ; Cc1cc2c3cc(-c4nc(-c5ccccc5)nc(-c5ccccc5)n4)ccc3c3cc4c(cc3c2cc1C)oc1ccccc14 ; Cc1cc2c3cc(-c4nc(-c5ccccc5)nc(-c5ccccc5)n4)ccc3c3ccc4c5ccccc5oc4c3c2cc1C]

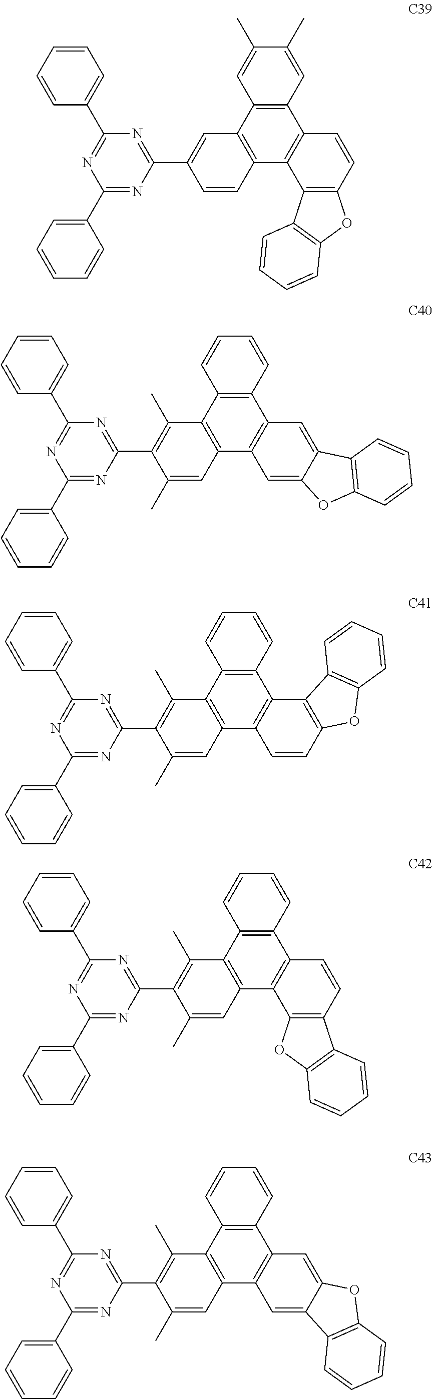

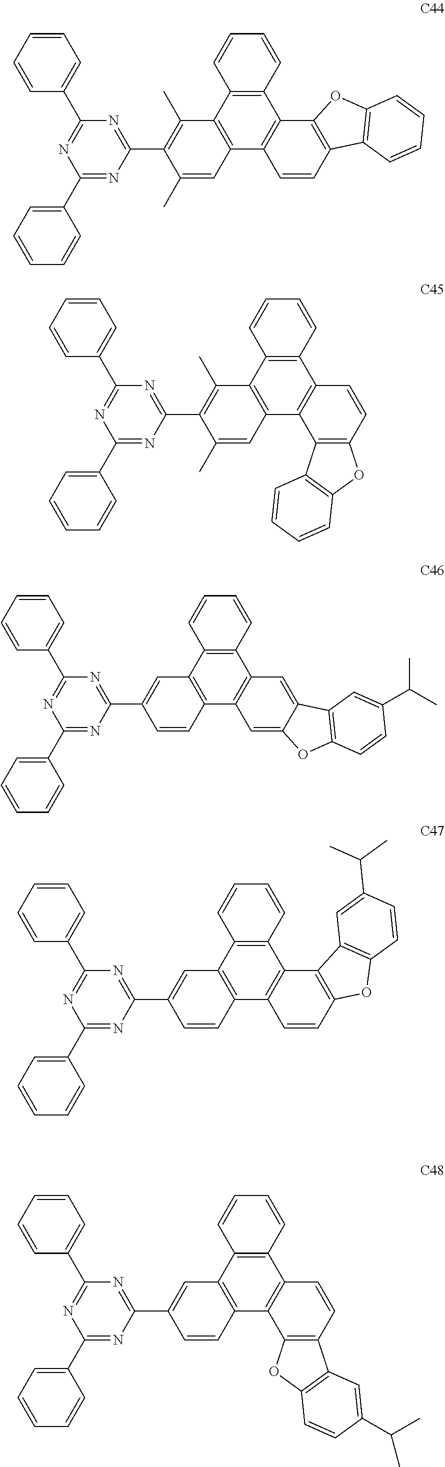

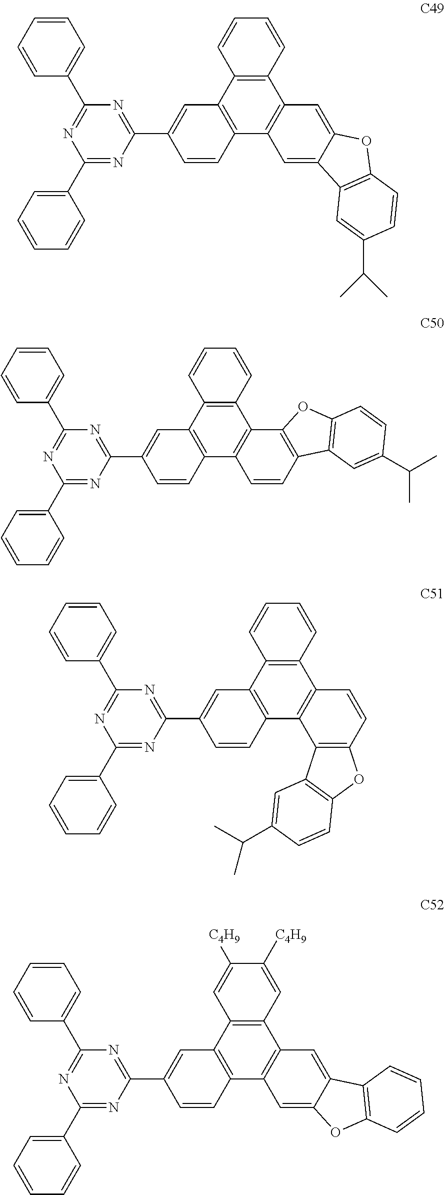

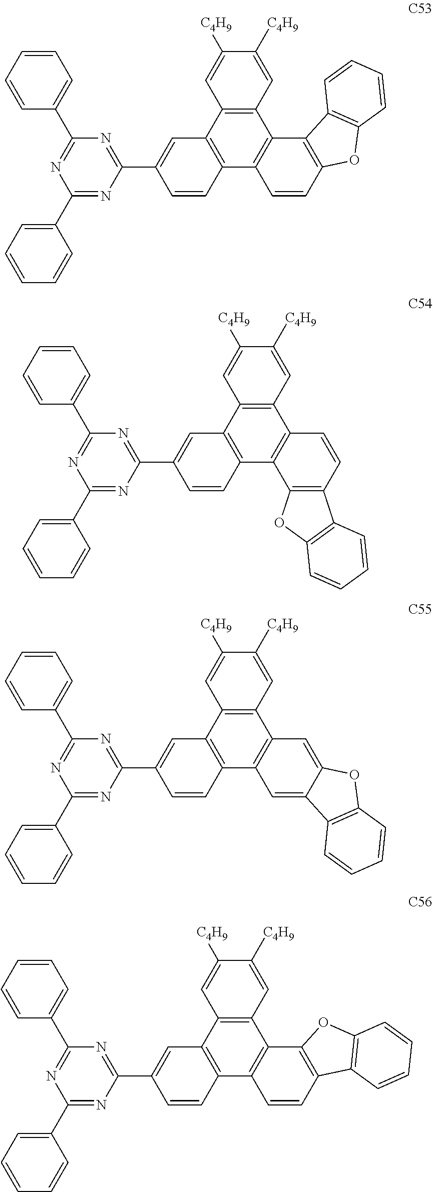

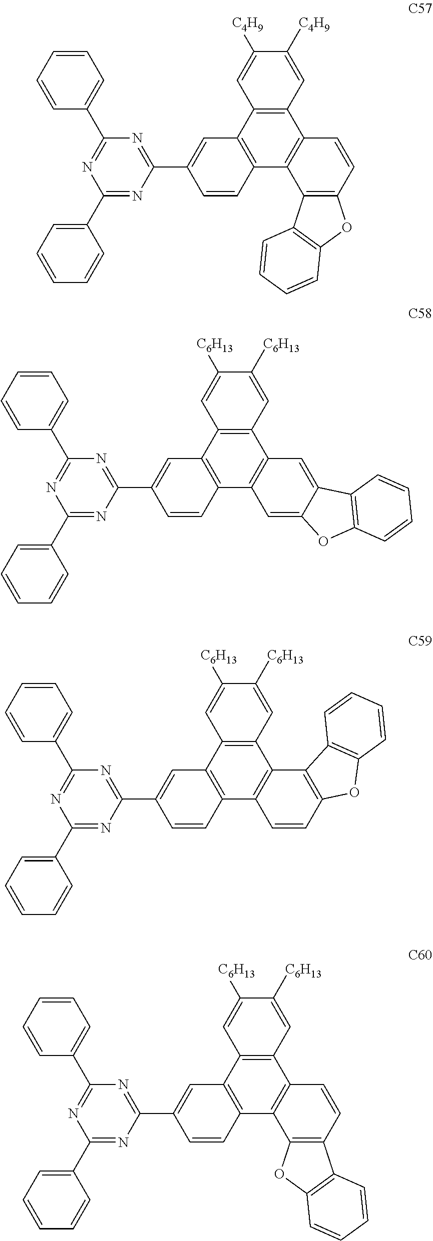

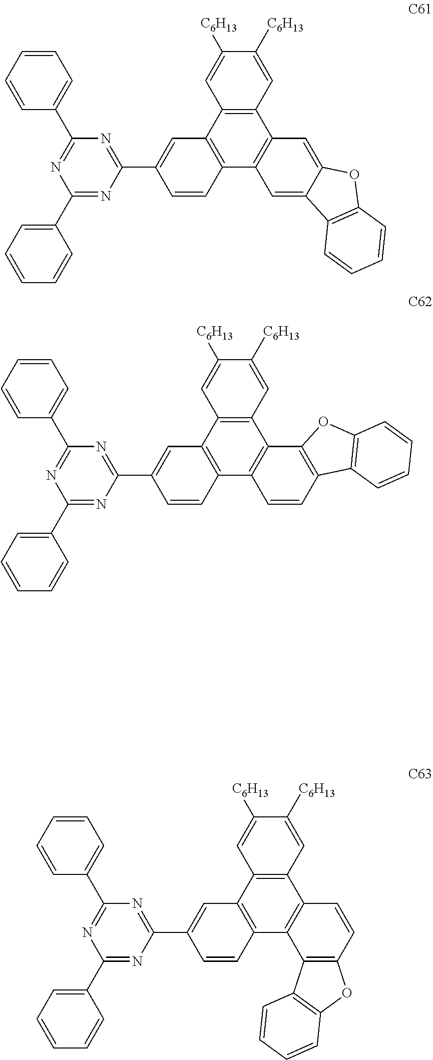

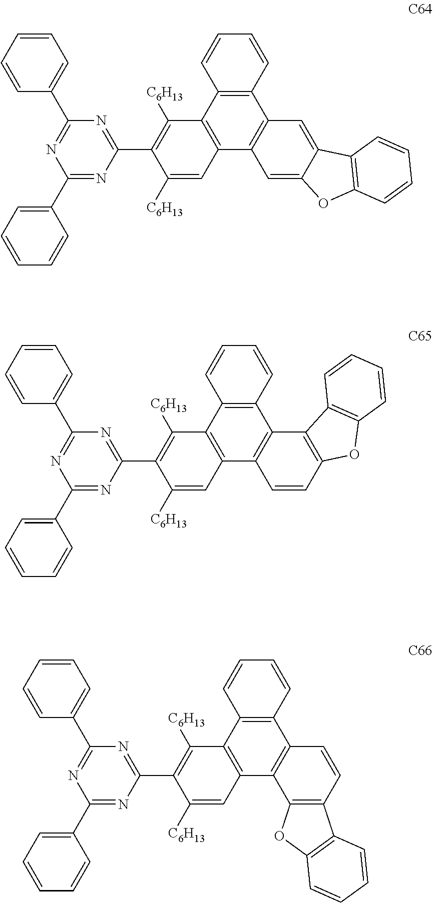

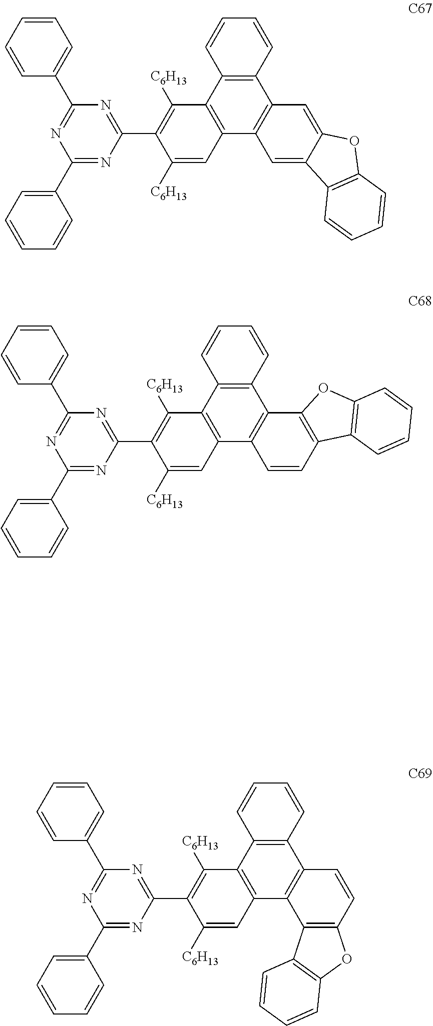

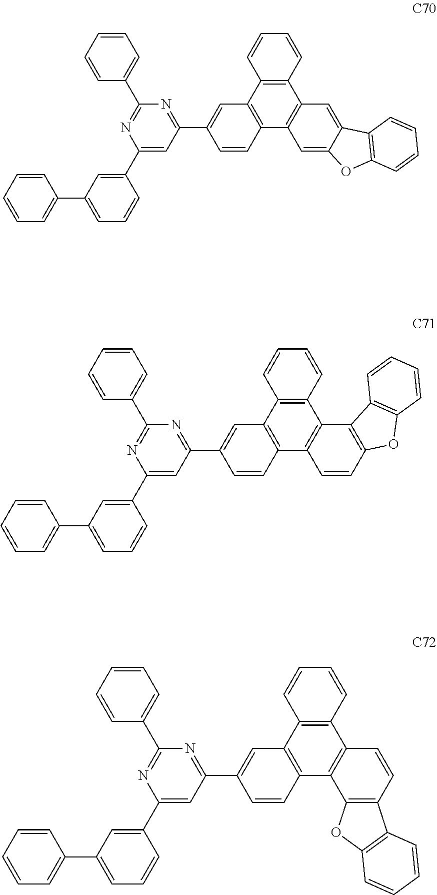 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4ccc6c7ccccc7oc6c45)nc(-c4ccccc4)n3)c2)cc1]

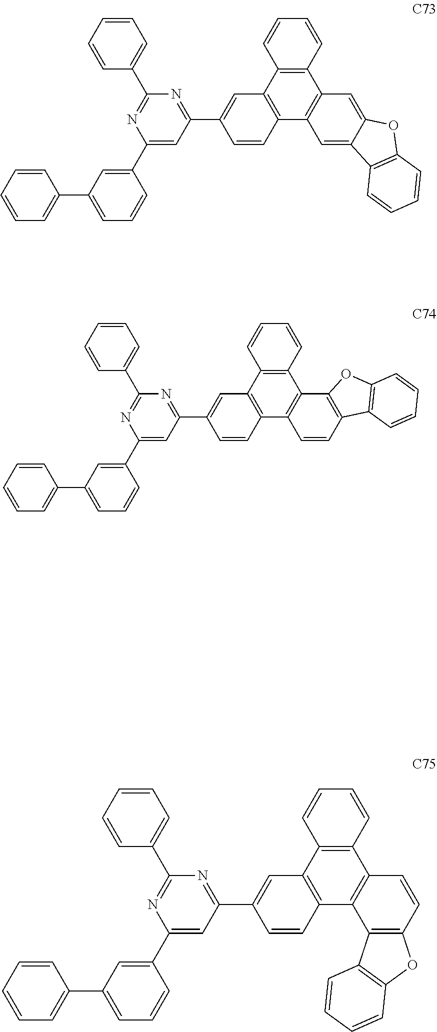 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

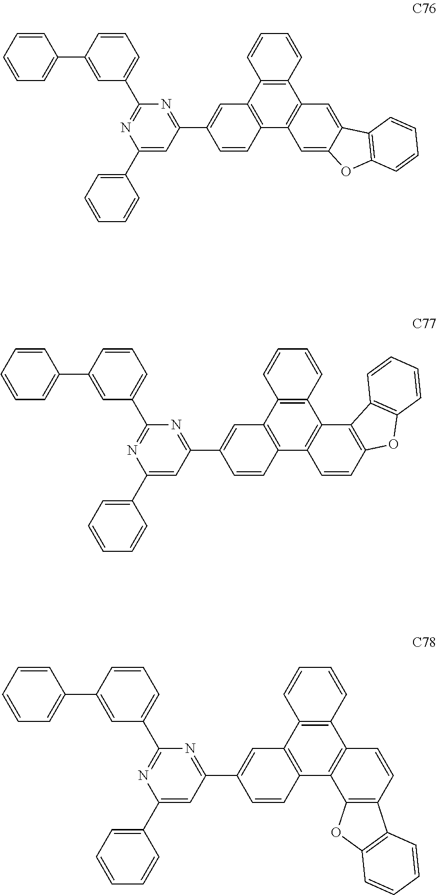 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

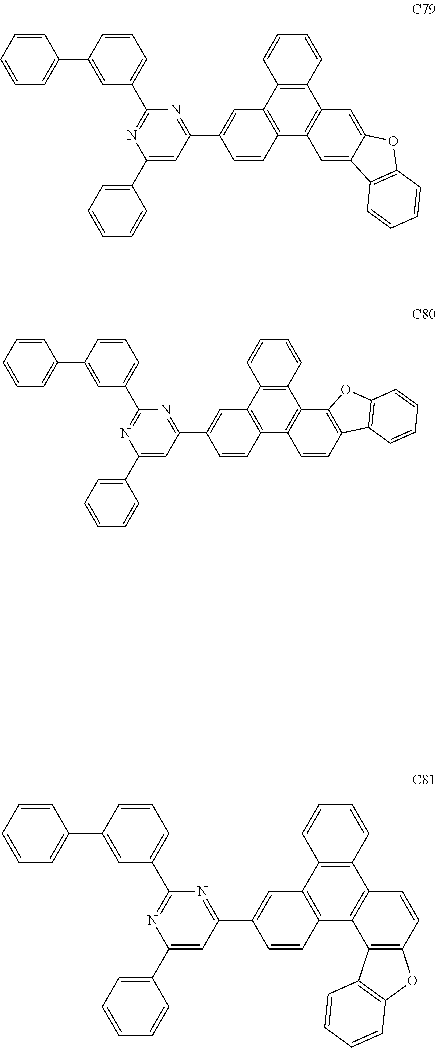 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

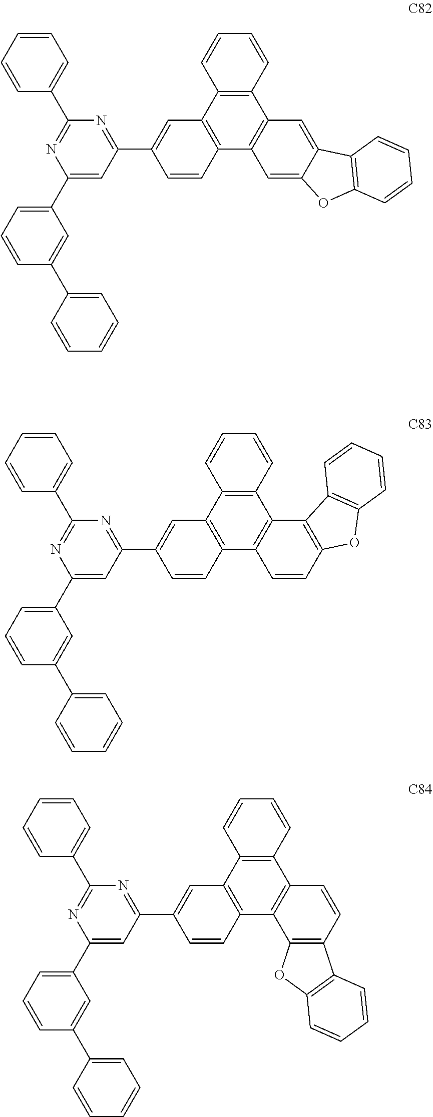 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; c1ccc(-c2cccc(-c3cc(-c4ccc5c(c4)c4ccccc4c4ccc6c7ccccc7oc6c45)nc(-c4ccccc4)n3)c2)cc1]

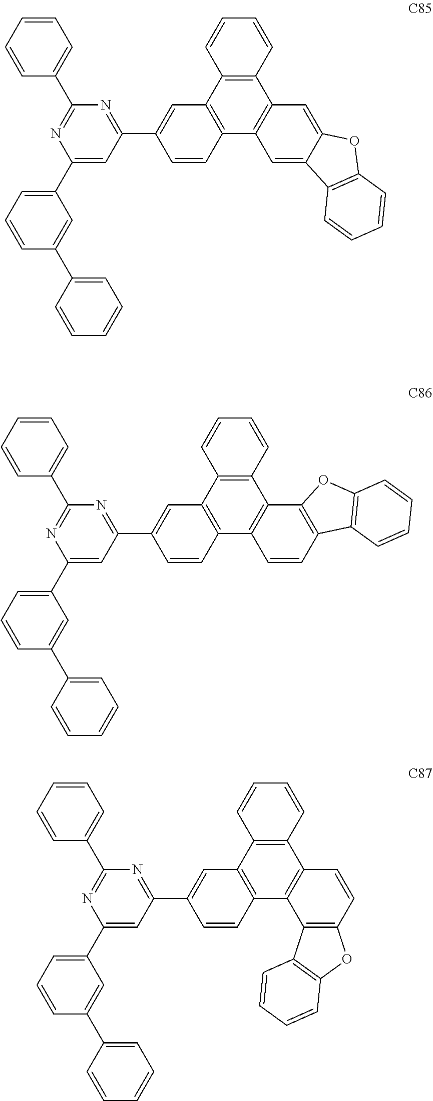 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

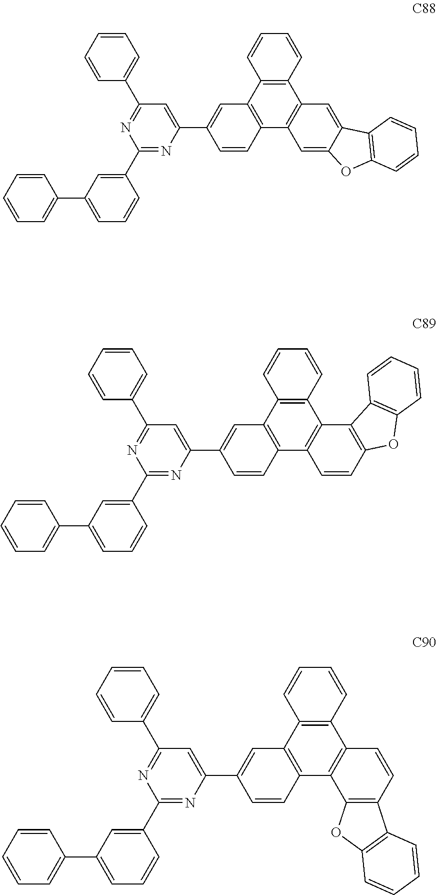 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

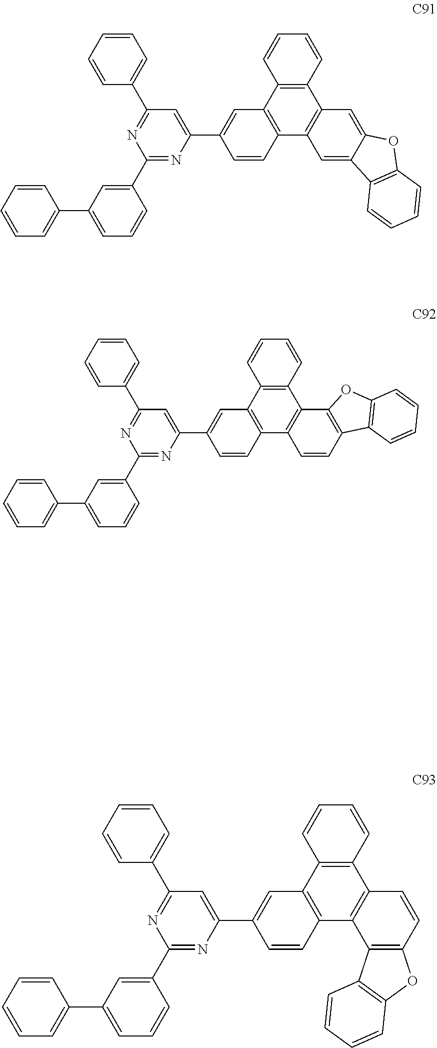 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

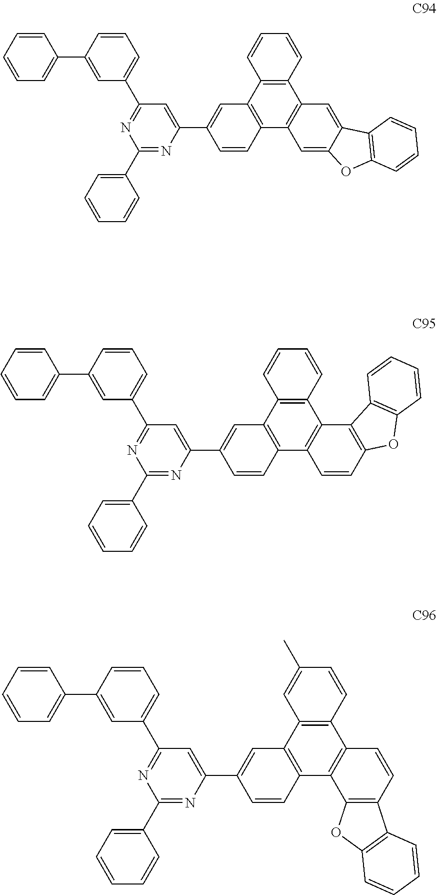 [SMILES: c1ccc(-c2cccc(-c3cc(-c4ccc5c(c4)c4ccccc4c4cc6c(cc54)oc4ccccc46)nc(-c4ccccc4)n3)c2)cc1 ; c1ccc(-c2cccc(-c3cc(-c4ccc5c(c4)c4ccccc4c4c5ccc5oc6ccccc6c54)nc(-c4ccccc4)n3)c2)cc1 ; Cc1ccc2c(c1)c1cc(-c3cc(-c4cccc(-c5ccccc5)c4)nc(-c4ccccc4)n3)ccc1c1c2ccc2c3ccccc3oc21]

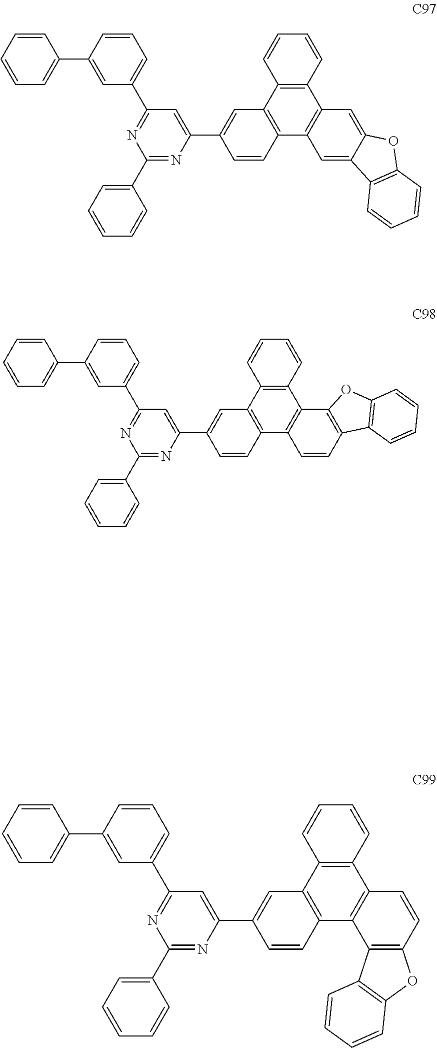 [SMILES: c1ccc(-c2cccc(-c3cc(-c4ccc5c(c4)c4ccccc4c4cc6oc7ccccc7c6cc54)nc(-c4ccccc4)n3)c2)cc1 ; c1ccc(-c2cccc(-c3cc(-c4ccc5c(c4)c4ccccc4c4c5ccc5c6ccccc6oc54)nc(-c4ccccc4)n3)c2)cc1 ; c1ccc(-c2cccc(-c3cc(-c4ccc5c(c4)c4ccccc4c4ccc6oc7ccccc7c6c45)nc(-c4ccccc4)n3)c2)cc1]

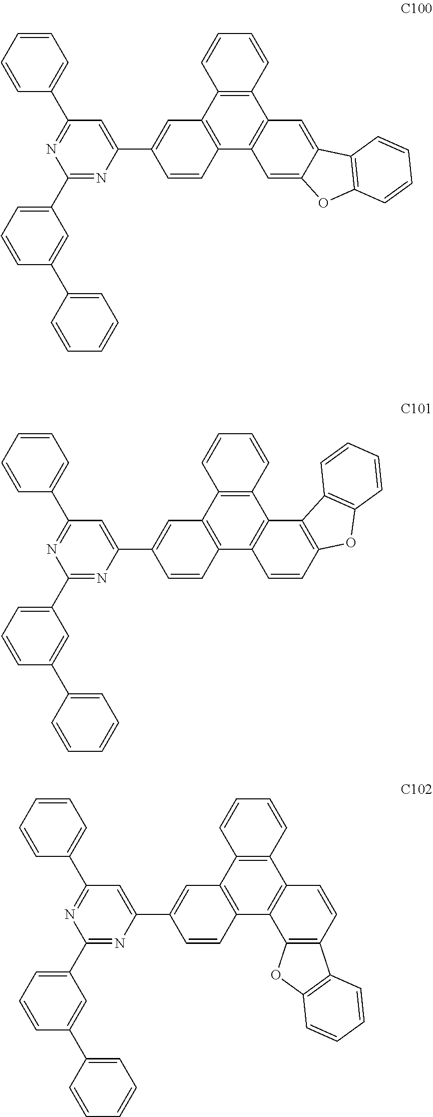 [SMILES: c1ccc(-c2cccc(-c3nc(-c4ccccc4)cc(-c4ccc5c(c4)c4ccccc4c4cc6c(cc54)oc4ccccc46)n3)c2)cc1 ; c1ccc(-c2cccc(-c3nc(-c4ccccc4)cc(-c4ccc5c(c4)c4ccccc4c4c5ccc5oc6ccccc6c54)n3)c2)cc1 ; c1ccc(-c2cccc(-c3nc(-c4ccccc4)cc(-c4ccc5c(c4)c4ccccc4c4ccc6c7ccccc7oc6c45)n3)c2)cc1]

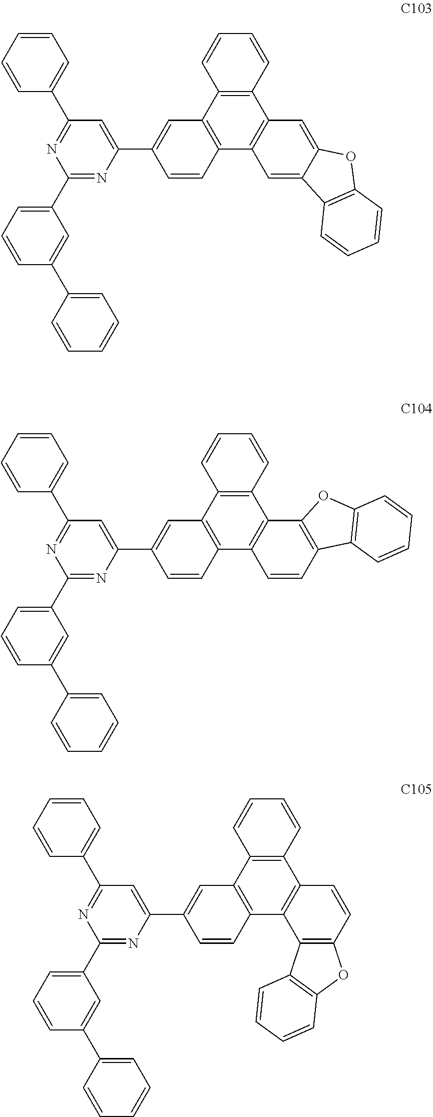 [SMILES: c1ccc(-c2cccc(-c3nc(-c4ccccc4)cc(-c4ccc5c(c4)c4ccccc4c4cc6oc7ccccc7c6cc54)n3)c2)cc1 ; c1ccc(-c2cccc(-c3nc(-c4ccccc4)cc(-c4ccc5c(c4)c4ccccc4c4c5ccc5c6ccccc6oc54)n3)c2)cc1 ; c1ccc(-c2cccc(-c3nc(-c4ccccc4)cc(-c4ccc5c(c4)c4ccccc4c4ccc6oc7ccccc7c6c45)n3)c2)cc1]

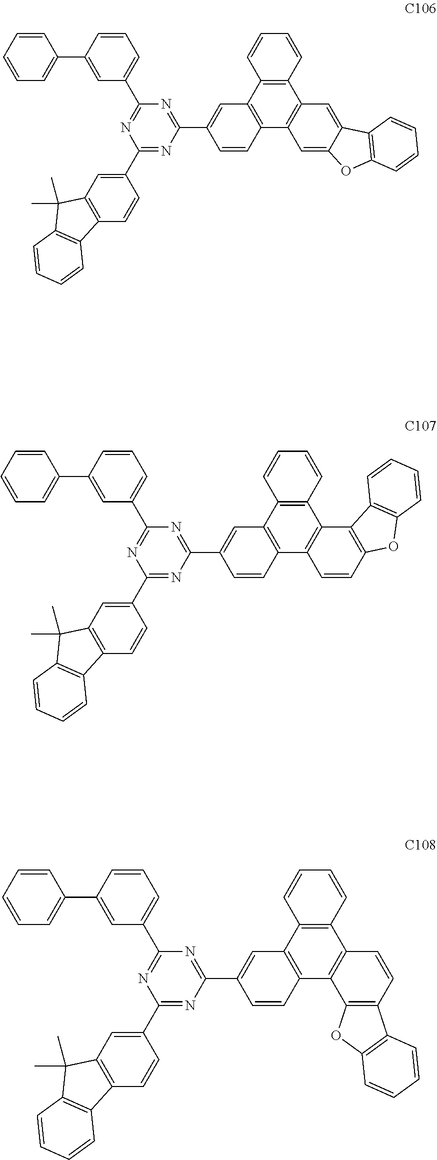 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4c5ccc5oc6ccccc6c54)n3)cc21 ; CC1(C)c2ccccc2-c2ccc(-c3nc(-c4cccc(-c5ccccc5)c4)nc(-c4ccc5c(c4)c4ccccc4c4ccc6c7ccccc7oc6c45)n3)cc21]

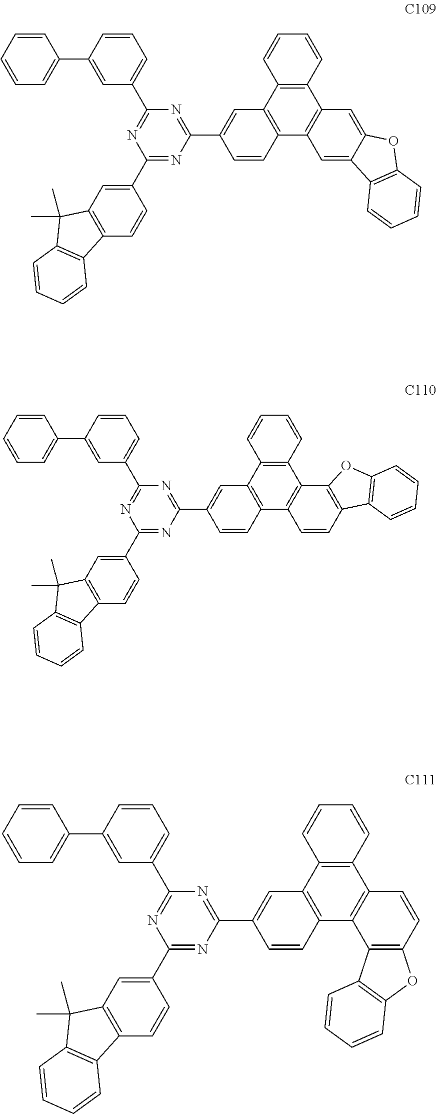 [SMILES: CC1(C)c2ccccc2-c2ccc(-c3nc(-c4cccc(-c5ccccc5)c4)nc(-c4ccc5c(c4)c4ccccc4c4cc6oc7ccccc7c6cc54)n3)cc21 ; CC1(C)c2ccccc2-c2ccc(-c3nc(-c4cccc(-c5ccccc5)c4)nc(-c4ccc5c(c4)c4ccccc4c4c5ccc5c6ccccc6oc54)n3)cc21 ; CC1(C)c2ccccc2-c2ccc(-c3nc(-c4cccc(-c5ccccc5)c4)nc(-c4ccc5c(c4)c4ccccc4c4ccc6oc7ccccc7c6c45)n3)cc21]

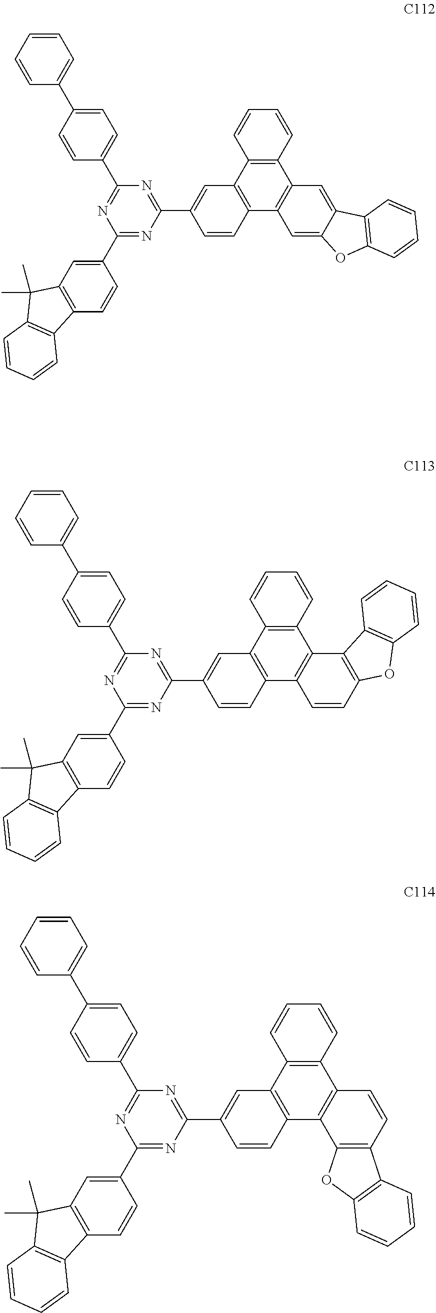 [SMILES: CC1(C)c2ccccc2-c2ccc(-c3nc(-c4ccc(-c5ccccc5)cc4)nc(-c4ccc5c(c4)c4ccccc4c4cc6c(cc54)oc4ccccc46)n3)cc21 ; CC1(C)c2ccccc2-c2ccc(-c3nc(-c4ccc(-c5ccccc5)cc4)nc(-c4ccc5c(c4)c4ccccc4c4c5ccc5oc6ccccc6c54)n3)cc21 ; CC1(C)c2ccccc2-c2ccc(-c3nc(-c4ccc(-c5ccccc5)cc4)nc(-c4ccc5c(c4)c4ccccc4c4ccc6c7ccccc7oc6c45)n3)cc21]

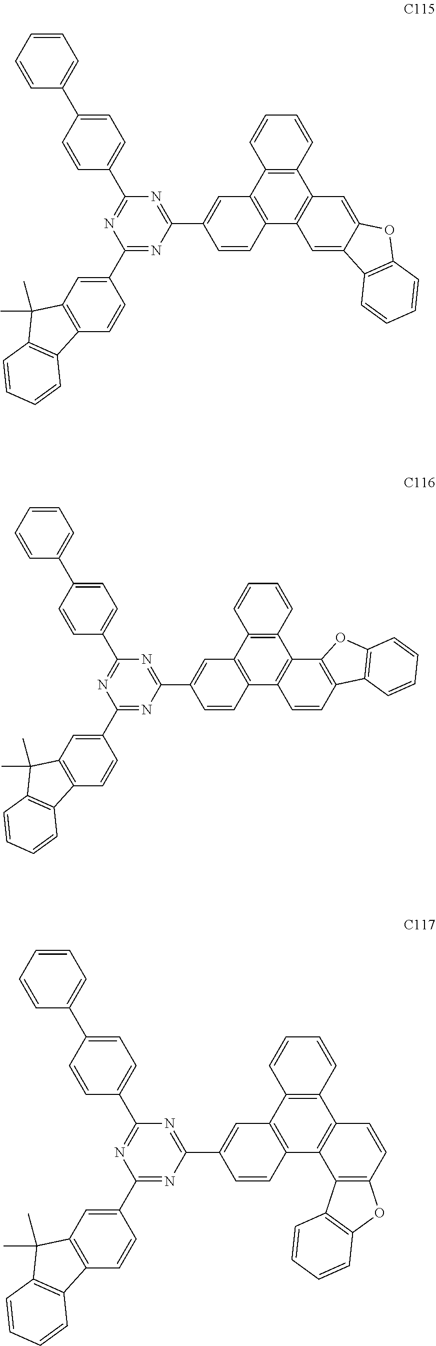 [SMILES: CC1(C)c2ccccc2-c2ccc(-c3nc(-c4ccc(-c5ccccc5)cc4)nc(-c4ccc5c(c4)c4ccccc4c4cc6oc7ccccc7c6cc54)n3)cc21 ; CC1(C)c2ccccc2-c2ccc(-c3nc(-c4ccc(-c5ccccc5)cc4)nc(-c4ccc5c(c4)c4ccccc4c4c5ccc5c6ccccc6oc54)n3)cc21 ; CC1(C)c2ccccc2-c2ccc(-c3nc(-c4ccc(-c5ccccc5)cc4)nc(-c4ccc5c(c4)c4ccccc4c4ccc6oc7ccccc7c6c45)n3)cc21]

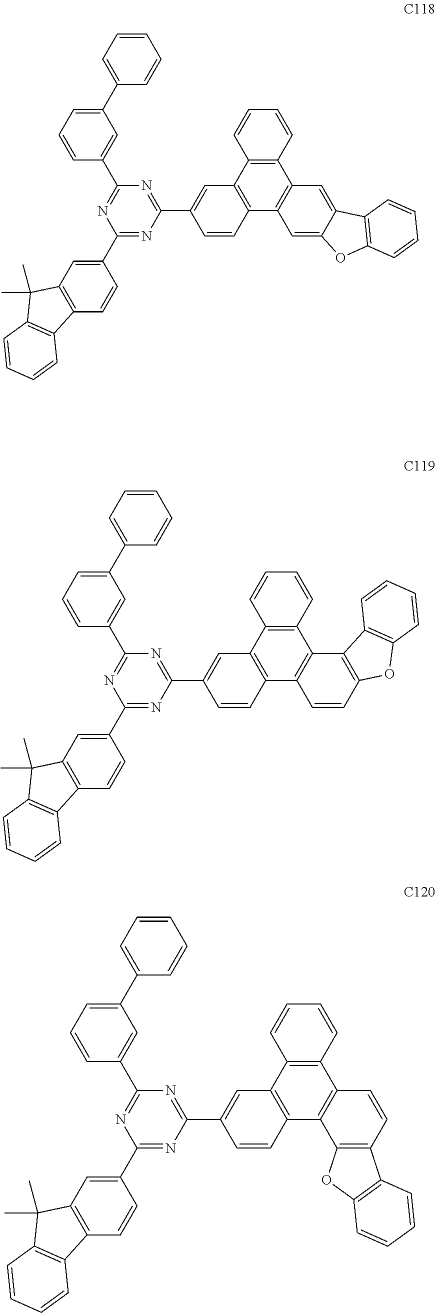 [SMILES: CC1(C)c2ccccc2-c2ccc(-c3nc(-c4cccc(-c5ccccc5)c4)nc(-c4ccc5c(c4)c4ccccc4c4cc6c(cc54)oc4ccccc46)n3)cc21 ; CC1(C)c2ccccc2-c2ccc(-c3nc(-c4cccc(-c5ccccc5)c4)nc(-c4ccc5c(c4)c4ccccc4c4c5ccc5oc6ccccc6c54)n3)cc21 ; CC1(C)c2ccccc2-c2ccc(-c3nc(-c4cccc(-c5ccccc5)c4)nc(-c4ccc5c(c4)c4ccccc4c4ccc6c7ccccc7oc6c45)n3)cc21]

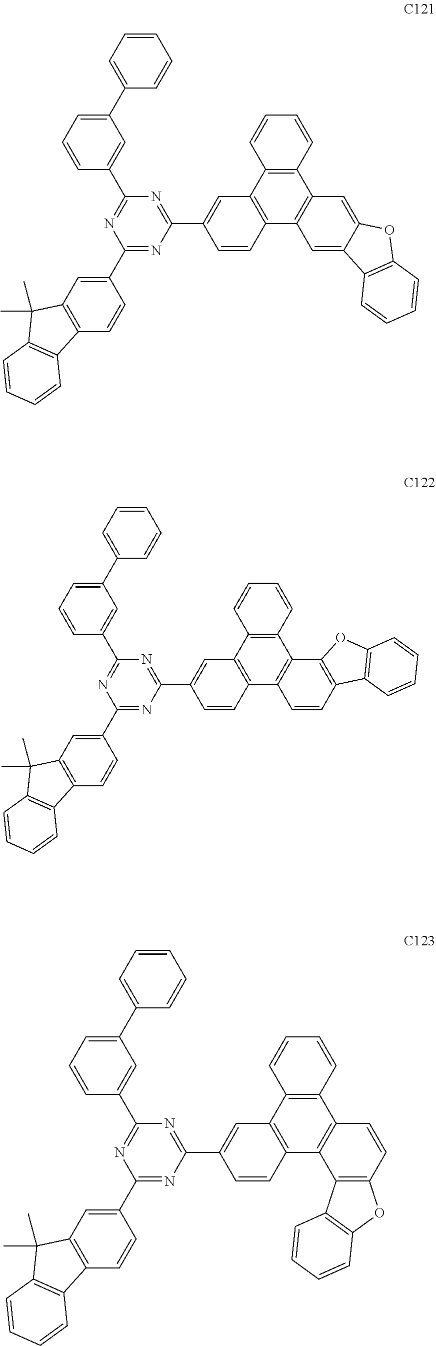 [SMILES: CC1(C)c2ccccc2-c2ccc(-c3nc(-c4cccc(-c5ccccc5)c4)nc(-c4ccc5c(c4)c4ccccc4c4cc6oc7ccccc7c6cc54)n3)cc21 ; CC1(C)c2ccccc2-c2ccc(-c3nc(-c4cccc(-c5ccccc5)c4)nc(-c4ccc5c(c4)c4ccccc4c4c5ccc5c6ccccc6oc54)n3)cc21 ; CC1(C)c2ccccc2-c2ccc(-c3nc(-c4cccc(-c5ccccc5)c4)nc(-c4ccc5c(c4)c4ccccc4c4ccc6oc7ccccc7c6c45)n3)cc21]

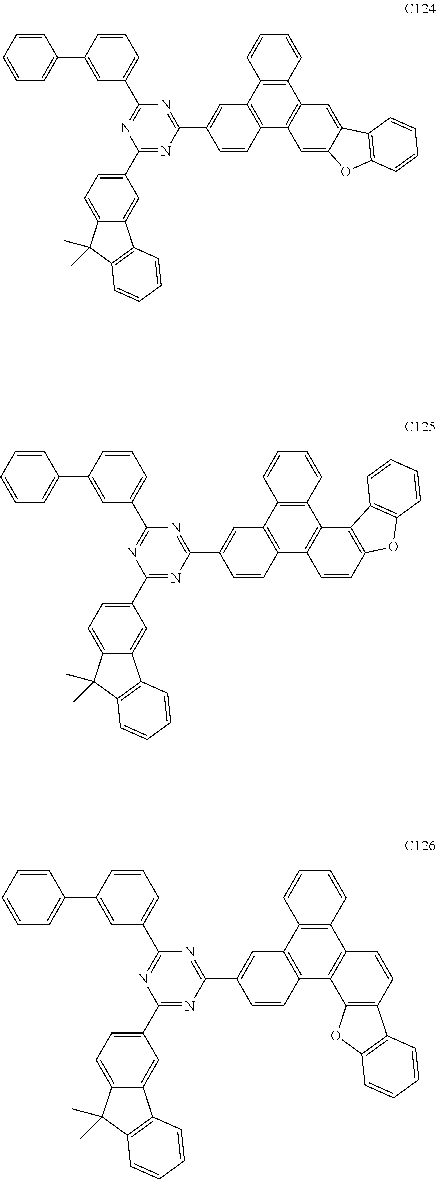 [SMILES: CC1(C)c2ccccc2-c2cc(-c3nc(-c4cccc(-c5ccccc5)c4)nc(-c4ccc5c(c4)c4ccccc4c4cc6c(cc54)oc4ccccc46)n3)ccc21 ; CC1(C)c2ccccc2-c2cc(-c3nc(-c4cccc(-c5ccccc5)c4)nc(-c4ccc5c(c4)c4ccccc4c4c5ccc5oc6ccccc6c54)n3)ccc21 ; CC1(C)c2ccccc2-c2cc(-c3nc(-c4cccc(-c5ccccc5)c4)nc(-c4ccc5c(c4)c4ccccc4c4ccc6c7ccccc7oc6c45)n3)ccc21]

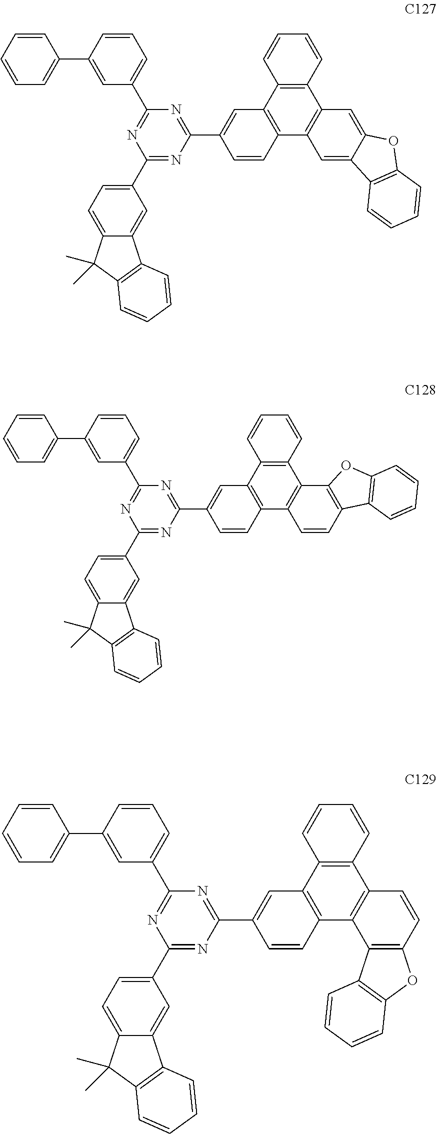 [SMILES: CC1(C)c2ccccc2-c2cc(-c3nc(-c4cccc(-c5ccccc5)c4)nc(-c4ccc5c(c4)c4ccccc4c4cc6oc7ccccc7c6cc54)n3)ccc21 ; CC1(C)c2ccccc2-c2cc(-c3nc(-c4cccc(-c5ccccc5)c4)nc(-c4ccc5c(c4)c4ccccc4c4c5ccc5c6ccccc6oc54)n3)ccc21 ; CC1(C)c2ccccc2-c2cc(-c3nc(-c4cccc(-c5ccccc5)c4)nc(-c4ccc5c(c4)c4ccccc4c4ccc6oc7ccccc7c6c45)n3)ccc21]

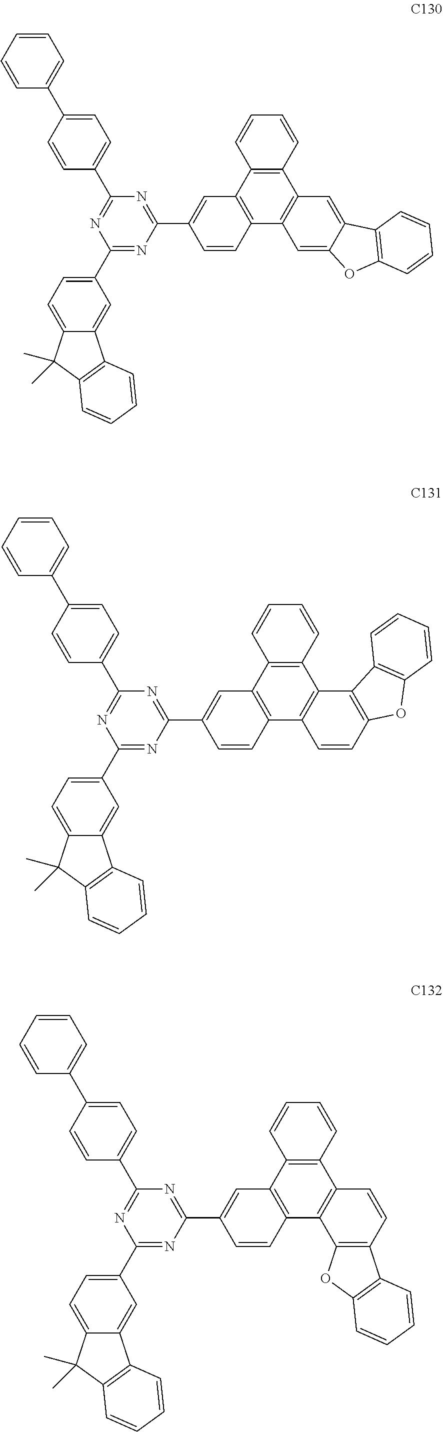 [SMILES: CC1(C)c2ccccc2-c2cc(-c3nc(-c4ccc(-c5ccccc5)cc4)nc(-c4ccc5c(c4)c4ccccc4c4cc6c(cc54)oc4ccccc46)n3)ccc21 ; CC1(C)c2ccccc2-c2cc(-c3nc(-c4ccc(-c5ccccc5)cc4)nc(-c4ccc5c(c4)c4ccccc4c4c5ccc5oc6ccccc6c54)n3)ccc21 ; CC1(C)c2ccccc2-c2cc(-c3nc(-c4ccc(-c5ccccc5)cc4)nc(-c4ccc5c(c4)c4ccccc4c4ccc6c7ccccc7oc6c45)n3)ccc21]

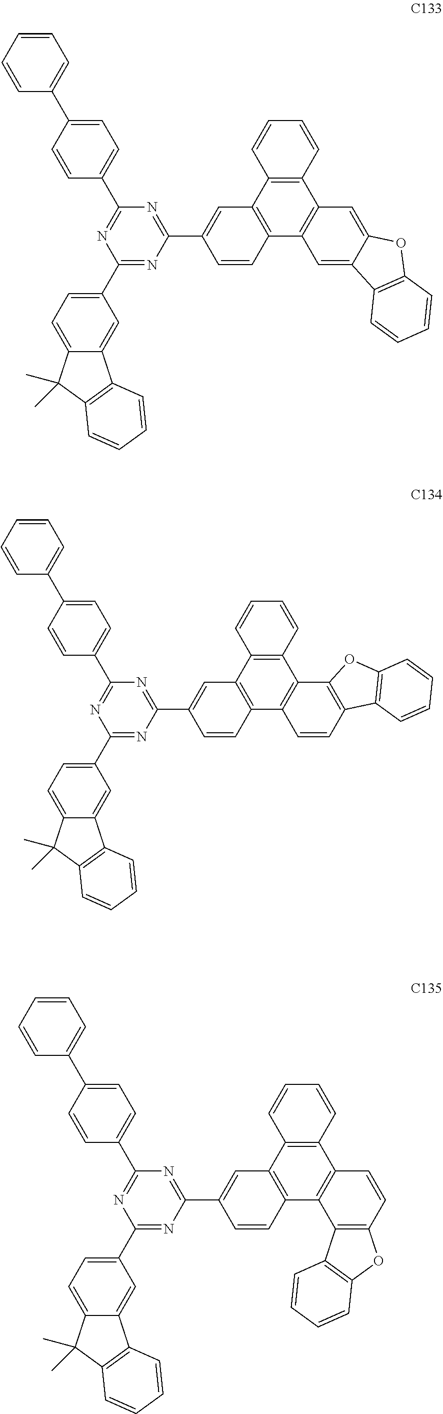 [SMILES: CC1(C)c2ccccc2-c2cc(-c3nc(-c4ccc(-c5ccccc5)cc4)nc(-c4ccc5c(c4)c4ccccc4c4cc6oc7ccccc7c6cc54)n3)ccc21 ; CC1(C)c2ccccc2-c2cc(-c3nc(-c4ccc(-c5ccccc5)cc4)nc(-c4ccc5c(c4)c4ccccc4c4c5ccc5c6ccccc6oc54)n3)ccc21 ; CC1(C)c2ccccc2-c2cc(-c3nc(-c4ccc(-c5ccccc5)cc4)nc(-c4ccc5c(c4)c4ccccc4c4ccc6oc7ccccc7c6c45)n3)ccc21]

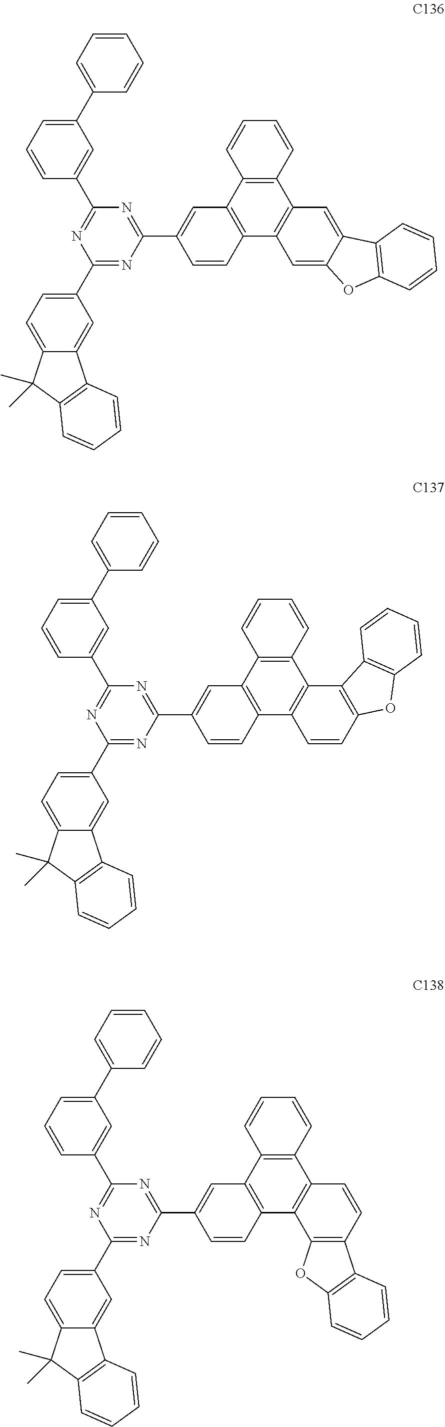 [SMILES: CC1(C)c2ccccc2-c2cc(-c3nc(-c4cccc(-c5ccccc5)c4)nc(-c4ccc5c(c4)c4ccccc4c4cc6c(cc54)oc4ccccc46)n3)ccc21 ; CC1(C)c2ccccc2-c2cc(-c3nc(-c4cccc(-c5ccccc5)c4)nc(-c4ccc5c(c4)c4ccccc4c4c5ccc5oc6ccccc6c54)n3)ccc21 ; CC1(C)c2ccccc2-c2cc(-c3nc(-c4cccc(-c5ccccc5)c4)nc(-c4ccc5c(c4)c4ccccc4c4ccc6c7ccccc7oc6c45)n3)ccc21]

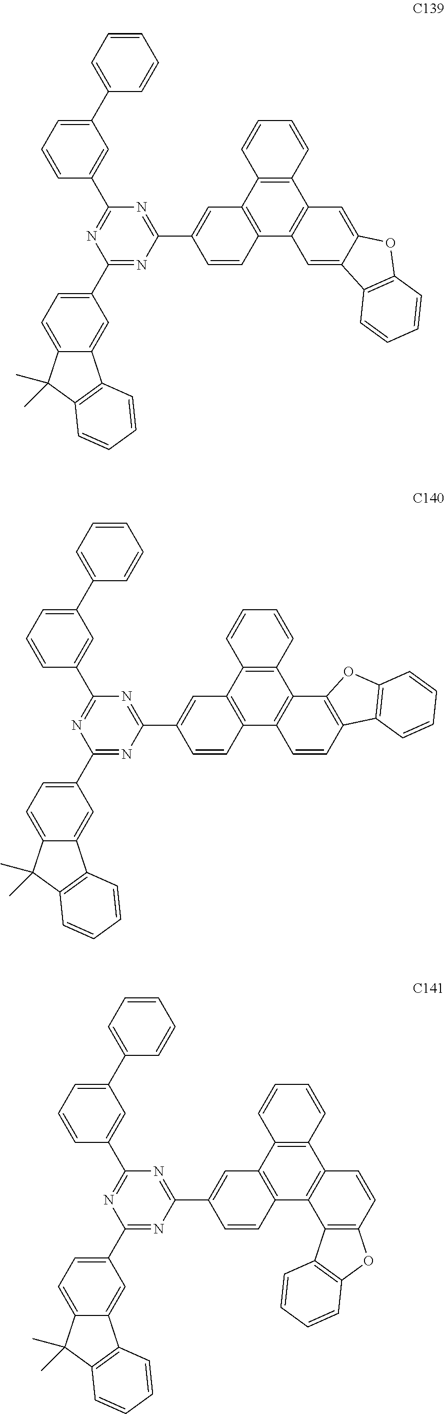 [SMILES: CC1(C)c2ccccc2-c2cc(-c3nc(-c4cccc(-c5ccccc5)c4)nc(-c4ccc5c(c4)c4ccccc4c4cc6oc7ccccc7c6cc54)n3)ccc21 ; CC1(C)c2ccccc2-c2cc(-c3nc(-c4cccc(-c5ccccc5)c4)nc(-c4ccc5c(c4)c4ccccc4c4c5ccc5c6ccccc6oc54)n3)ccc21 ; CC1(C)c2ccccc2-c2cc(-c3nc(-c4cccc(-c5ccccc5)c4)nc(-c4ccc5c(c4)c4ccccc4c4ccc6oc7ccccc7c6c45)n3)ccc21]

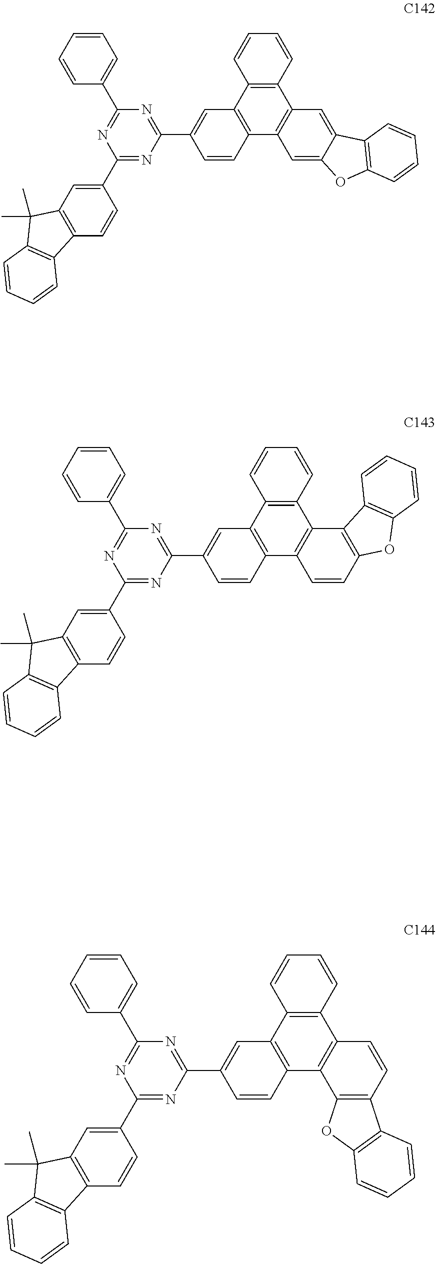 [SMILES: CC1(C)c2ccccc2-c2ccc(-c3nc(-c4ccccc4)nc(-c4ccc5c(c4)c4ccccc4c4cc6c(cc54)oc4ccccc46)n3)cc21 ; CC1(C)c2ccccc2-c2ccc(-c3nc(-c4ccccc4)nc(-c4ccc5c(c4)c4ccccc4c4c5ccc5oc6ccccc6c54)n3)cc21 ; CC1(C)c2ccccc2-c2ccc(-c3nc(-c4ccccc4)nc(-c4ccc5c(c4)c4ccccc4c4ccc6c7ccccc7oc6c45)n3)cc21]

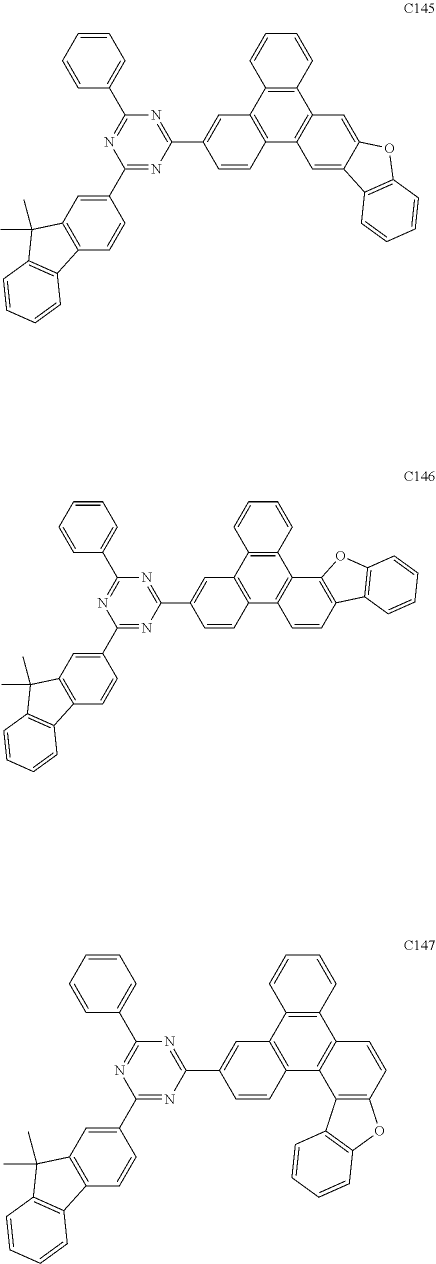 [SMILES: CC1(C)c2ccccc2-c2ccc(-c3nc(-c4ccccc4)nc(-c4ccc5c(c4)c4ccccc4c4cc6oc7ccccc7c6cc54)n3)cc21 ; CC1(C)c2ccccc2-c2ccc(-c3nc(-c4ccccc4)nc(-c4ccc5c(c4)c4ccccc4c4c5ccc5c6ccccc6oc54)n3)cc21 ; CC1(C)c2ccccc2-c2ccc(-c3nc(-c4ccccc4)nc(-c4ccc5c(c4)c4ccccc4c4ccc6oc7ccccc7c6c45)n3)cc21]

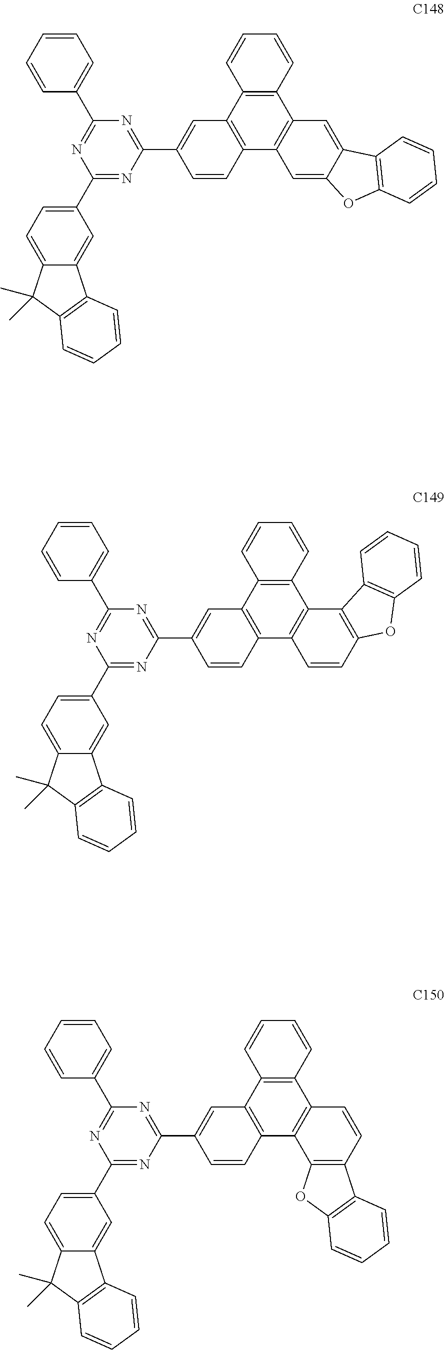 [SMILES: CC1(C)c2ccccc2-c2cc(-c3nc(-c4ccccc4)nc(-c4ccc5c(c4)c4ccccc4c4cc6c(cc54)oc4ccccc46)n3)ccc21 ; CC1(C)c2ccccc2-c2cc(-c3nc(-c4ccccc4)nc(-c4ccc5c(c4)c4ccccc4c4c5ccc5oc6ccccc6c54)n3)ccc21 ; CC1(C)c2ccccc2-c2cc(-c3nc(-c4ccccc4)nc(-c4ccc5c(c4)c4ccccc4c4ccc6c7ccccc7oc6c45)n3)ccc21]

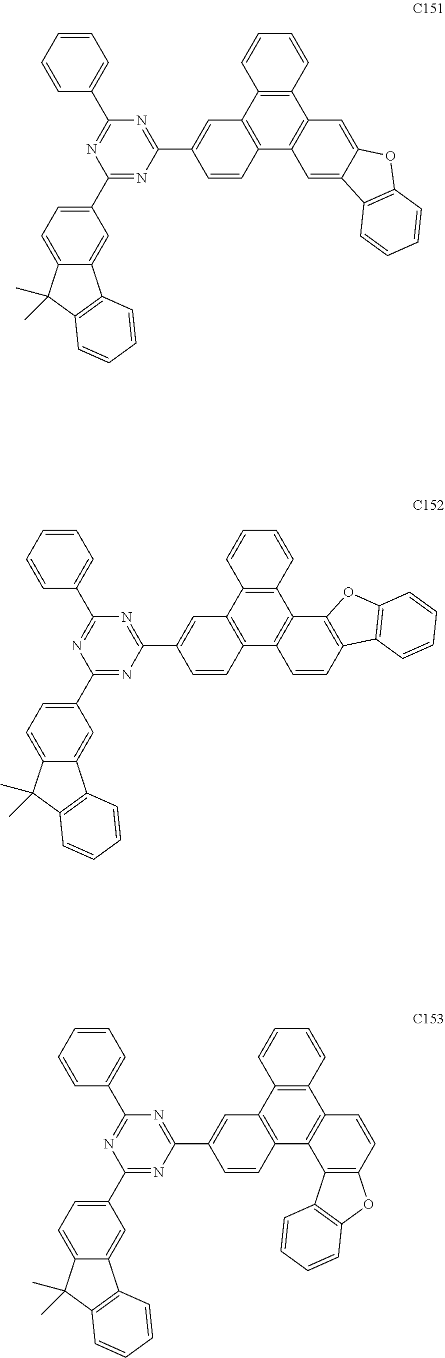 [SMILES: CC1(C)c2ccccc2-c2cc(-c3nc(-c4ccccc4)nc(-c4ccc5c(c4)c4ccccc4c4cc6oc7ccccc7c6cc54)n3)ccc21 ; CC1(C)c2ccccc2-c2cc(-c3nc(-c4ccccc4)nc(-c4ccc5c(c4)c4ccccc4c4c5ccc5c6ccccc6oc54)n3)ccc21 ; CC1(C)c2ccccc2-c2cc(-c3nc(-c4ccccc4)nc(-c4ccc5c(c4)c4ccccc4c4ccc6oc7ccccc7c6c45)n3)ccc21]

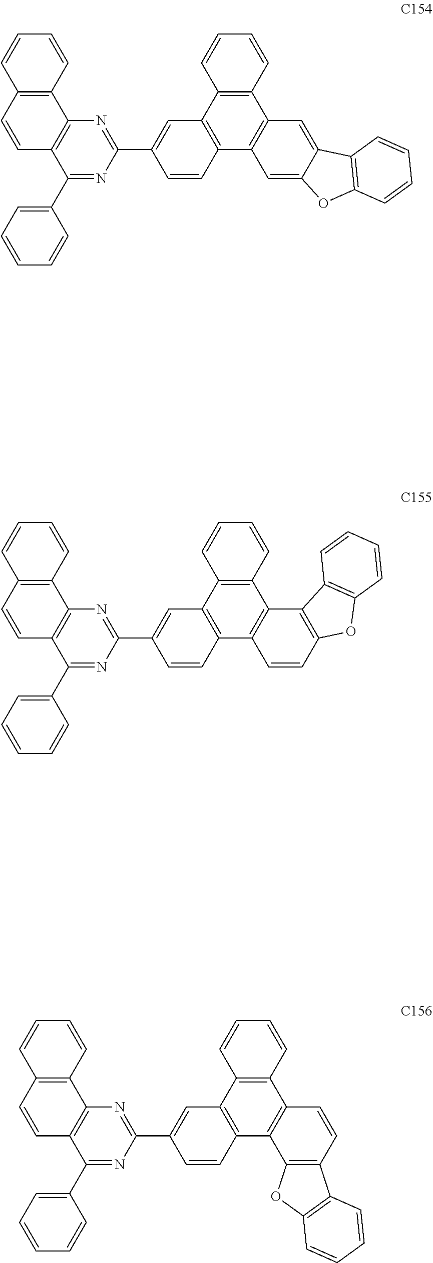 [SMILES: c1ccc(-c2nc(-c3ccc4c(c3)c3ccccc3c3cc5c(cc43)oc3ccccc35)nc3c2ccc2ccccc23)cc1 ; c1ccc(-c2nc(-c3ccc4c(c3)c3ccccc3c3c4ccc4oc5ccccc5c43)nc3c2ccc2ccccc23)cc1 ; c1ccc(-c2nc(-c3ccc4c(c3)c3ccccc3c3ccc5c6ccccc6oc5c34)nc3c2ccc2ccccc23)cc1]

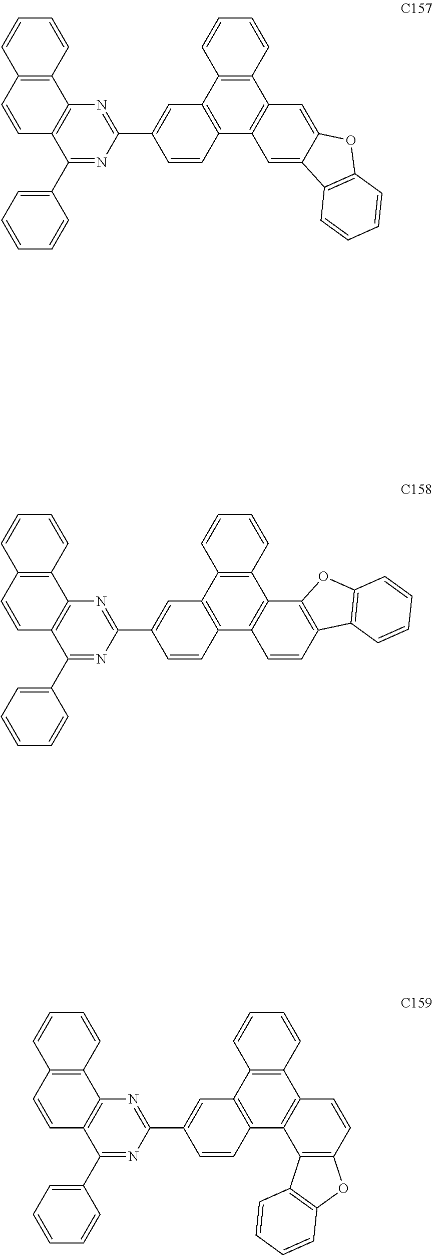 [SMILES: c1ccc(-c2nc(-c3ccc4c(c3)c3ccccc3c3cc5oc6ccccc6c5cc43)nc3c2ccc2ccccc23)cc1 ; c1ccc(-c2nc(-c3ccc4c(c3)c3ccccc3c3c4ccc4c5ccccc5oc43)nc3c2ccc2ccccc23)cc1 ; c1ccc(-c2nc(-c3ccc4c(c3)c3ccccc3c3ccc5oc6ccccc6c5c34)nc3c2ccc2ccccc23)cc1]

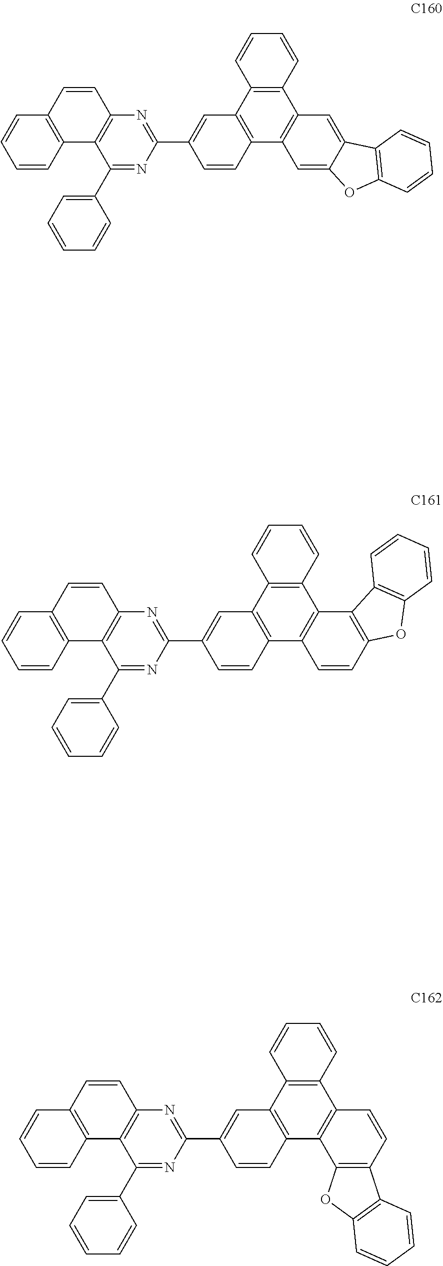 [SMILES: c1ccc(-c2nc(-c3ccc4c(c3)c3ccccc3c3cc5c(cc43)oc3ccccc35)nc3ccc4ccccc4c23)cc1 ; c1ccc(-c2nc(-c3ccc4c(c3)c3ccccc3c3c4ccc4oc5ccccc5c43)nc3ccc4ccccc4c23)cc1 ; c1ccc(-c2nc(-c3ccc4c(c3)c3ccccc3c3ccc5c6ccccc6oc5c34)nc3ccc4ccccc4c23)cc1]

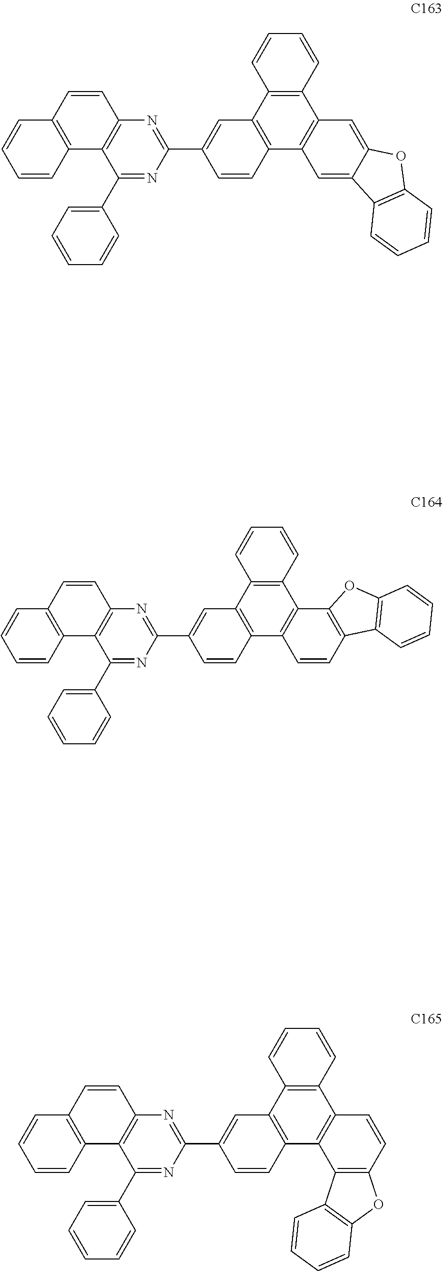 [SMILES: c1ccc(-c2nc(-c3ccc4c(c3)c3ccccc3c3cc5oc6ccccc6c5cc43)nc3ccc4ccccc4c23)cc1 ; c1ccc(-c2nc(-c3ccc4c(c3)c3ccccc3c3c4ccc4c5ccccc5oc43)nc3ccc4ccccc4c23)cc1 ; c1ccc(-c2nc(-c3ccc4c(c3)c3ccccc3c3ccc5oc6ccccc6c5c34)nc3ccc4ccccc4c23)cc1]

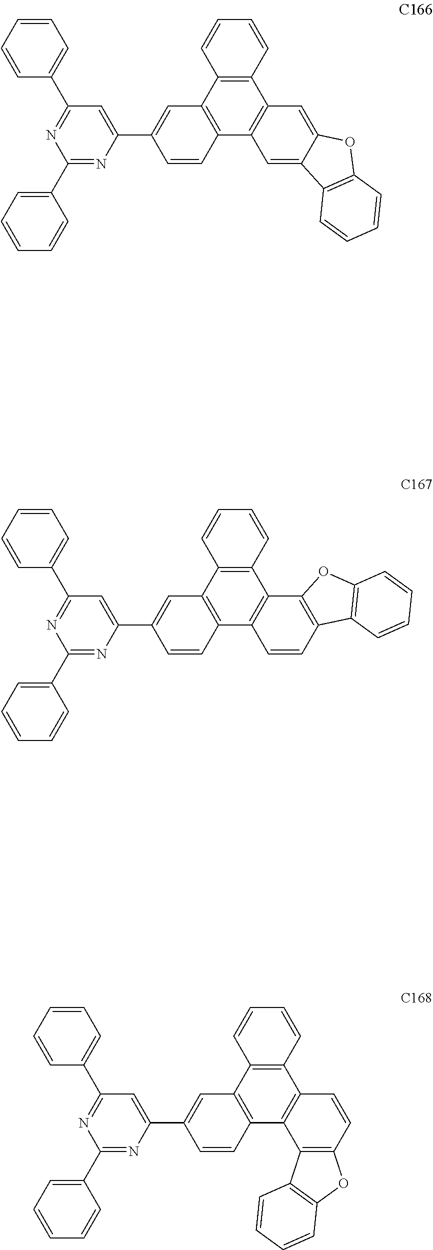 [SMILES: c1ccc(-c2cc(-c3ccc4c(c3)c3ccccc3c3cc5oc6ccccc6c5cc43)nc(-c3ccccc3)n2)cc1 ; c1ccc(-c2cc(-c3ccc4c(c3)c3ccccc3c3c4ccc4c5ccccc5oc43)nc(-c3ccccc3)n2)cc1 ; c1ccc(-c2cc(-c3ccc4c(c3)c3ccccc3c3ccc5oc6ccccc6c5c34)nc(-c3ccccc3)n2)cc1]

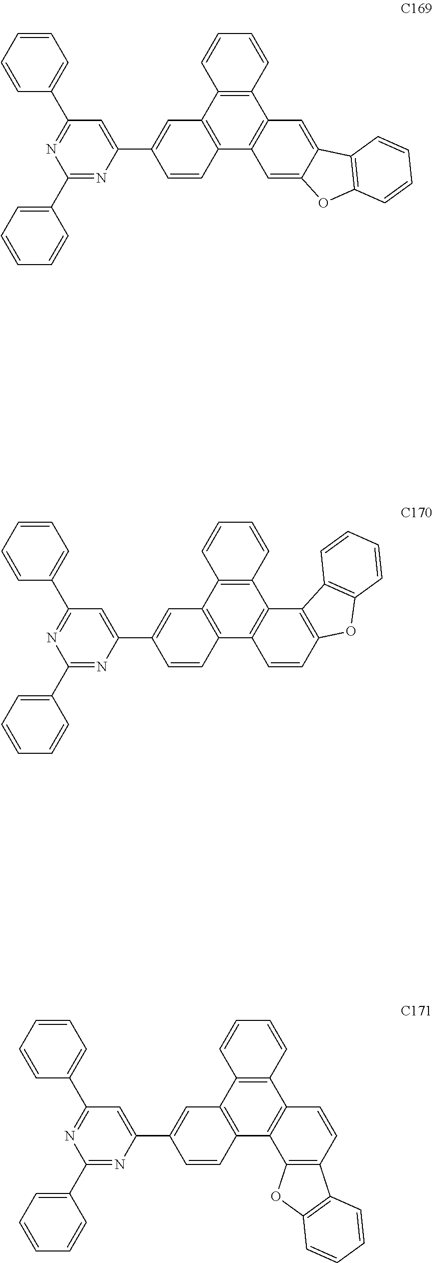 [SMILES: c1ccc(-c2cc(-c3ccc4c(c3)c3ccccc3c3cc5c(cc43)oc3ccccc35)nc(-c3ccccc3)n2)cc1 ; c1ccc(-c2cc(-c3ccc4c(c3)c3ccccc3c3c4ccc4oc5ccccc5c43)nc(-c3ccccc3)n2)cc1 ; c1ccc(-c2cc(-c3ccc4c(c3)c3ccccc3c3ccc5c6ccccc6oc5c34)nc(-c3ccccc3)n2)cc1]

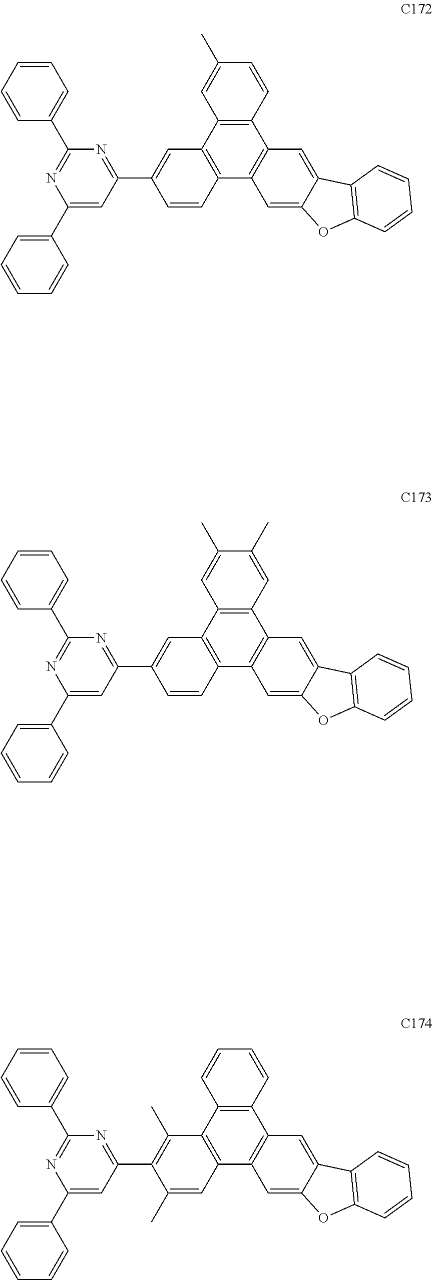 [SMILES: Cc1ccc2c(c1)c1cc(-c3cc(-c4ccccc4)nc(-c4ccccc4)n3)ccc1c1cc3oc4ccccc4c3cc21 ; Cc1cc2c3cc(-c4cc(-c5ccccc5)nc(-c5ccccc5)n4)ccc3c3cc4oc5ccccc5c4cc3c2cc1C ; Cc1cc2c3cc4oc5ccccc5c4cc3c3ccccc3c2c(C)c1-c1cc(-c2ccccc2)nc(-c2ccccc2)n1]

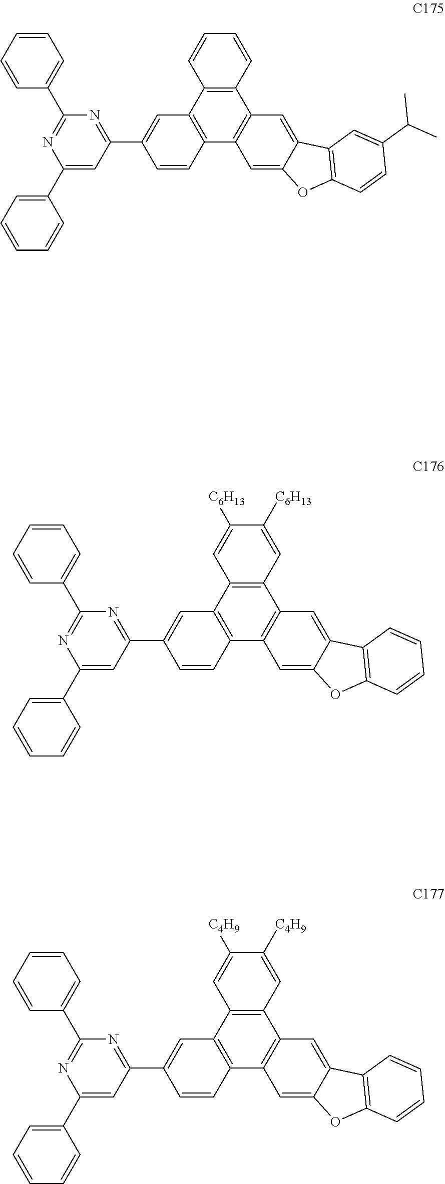

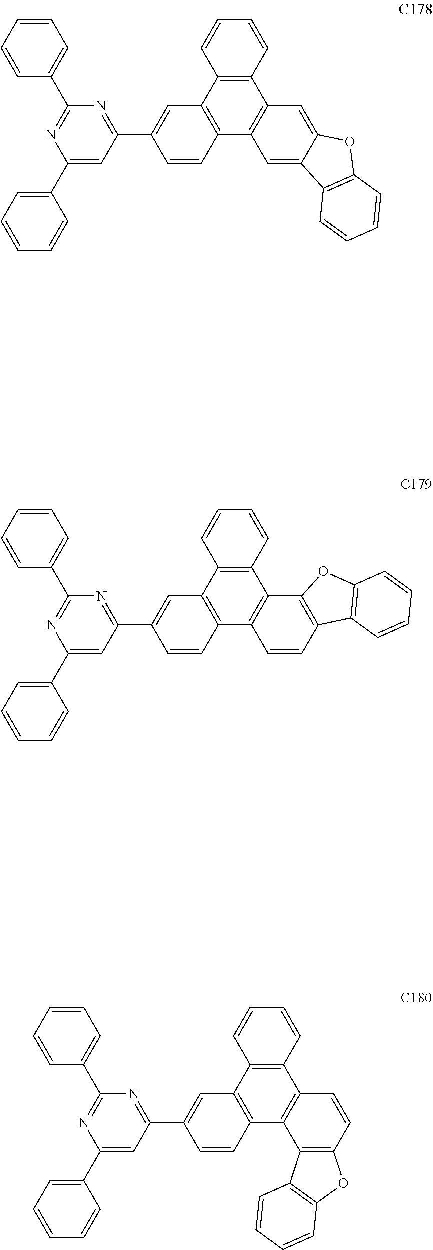 [SMILES: c1ccc(-c2cc(-c3ccc4c(c3)c3ccccc3c3cc5oc6ccccc6c5cc43)nc(-c3ccccc3)n2)cc1 ; c1ccc(-c2cc(-c3ccc4c(c3)c3ccccc3c3c4ccc4c5ccccc5oc43)nc(-c3ccccc3)n2)cc1 ; c1ccc(-c2cc(-c3ccc4c(c3)c3ccccc3c3ccc5oc6ccccc6c5c34)nc(-c3ccccc3)n2)cc1]

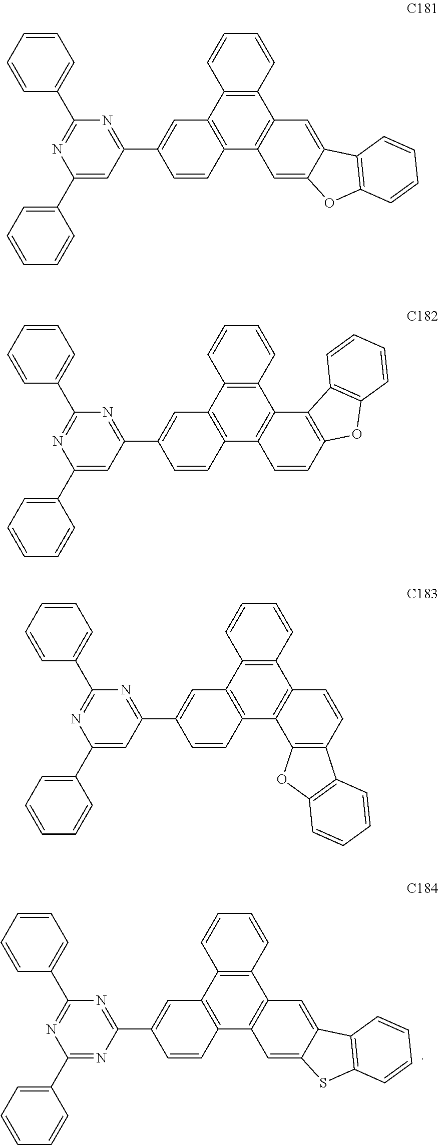 [SMILES: c1ccc(-c2cc(-c3ccc4c(c3)c3ccccc3c3cc5c(cc43)oc3ccccc35)nc(-c3ccccc3)n2)cc1 ; c1ccc(-c2cc(-c3ccc4c(c3)c3ccccc3c3c4ccc4oc5ccccc5c43)nc(-c3ccccc3)n2)cc1 ; c1ccc(-c2cc(-c3ccc4c(c3)c3ccccc3c3ccc5c6ccccc6oc5c34)nc(-c3ccccc3)n2)cc1 ; c1ccc(-c2nc(-c3ccccc3)nc(-c3ccc4c(c3)c3ccccc3c3cc5c(cc43)sc3ccccc35)n2)cc1]

6. The organic compound of claim 5, wherein the organic compound is in a blue organic light emitting device.

7. A blue organic light emitting diode, comprising a first electrode, a second electrode, and an organic thin film layer between the first electrode and the second electrode, wherein the organic thin film layer is selected from the group consisting of a light emitting layer, a hole blocking layer, an electron transport layer, and combinations thereof, wherein the first light emitting layer comprises a first host and a first guest, and wherein the organic thin film layer comprises the organic compound of claim 1.

8. The blue organic light emitting diode of claim 7, wherein the organic thin film layer is the electron transport layer.

9. The blue organic light emitting diode of claim 7, wherein the hole blocking layer and the electron transport layer both comprise the organic compound of claim 1.

10. The blue organic light emitting diode of claim 9, wherein the organic compound comprised by the hole blocking layer and the electron transport layer are the same.

11. A blue organic light emitting diode, comprising:
a substrate;
a first electrode on the substrate;
a hole injection layer, a hole transport layer, an electron blocking layer and a light emitting layer, formed on the first electrode, wherein the light emitting layer comprises a guest and a host;
an electron transport layer on the light emitting layer; and
a second electrode on the electron transport layer;
wherein the host comprises the organic compound of claim 1.

12. The blue organic light emitting diode of claim 11, wherein the first electrode is selected from the group consisting of indium tin oxide, indium zinc oxide, aluminum zinc oxide, and combinations thereof.

13. The blue organic light emitting diode of claim 11, wherein the second electrode is selected from the group consisting of aluminum, magnesium, calcium, silver, and combinations thereof.

14. The blue organic light emitting diode of claim 11, wherein the organic compound is selected from the group consisting of the following compounds:

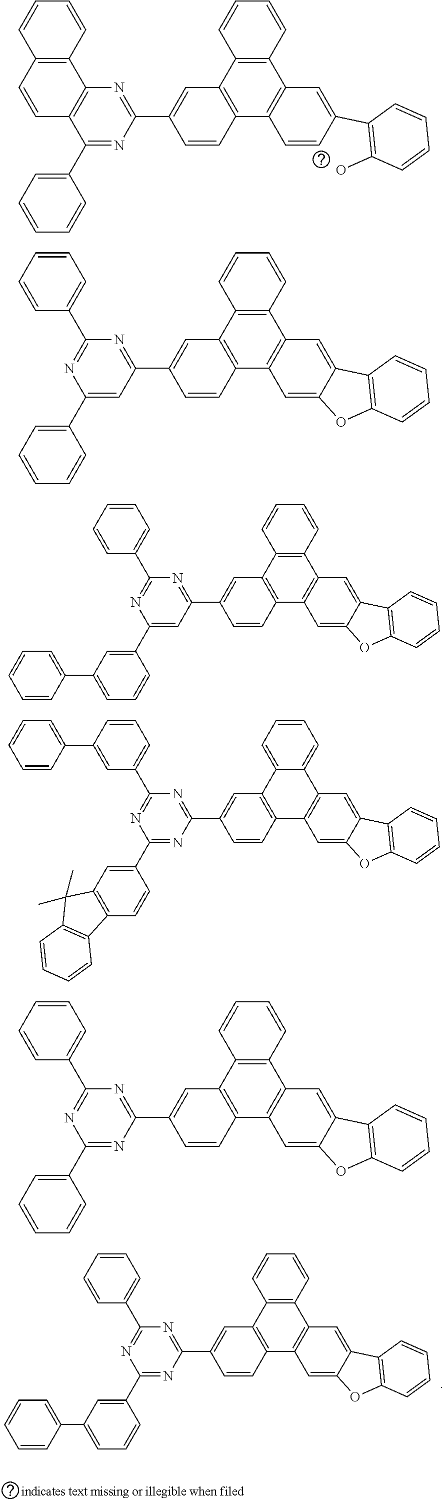

15. The blue organic light emitting diode of claim 11, further comprising a hole blocking layer between the light emitting layer and the electron transport layer.

16. The blue organic light emitting diode of claim 15, wherein the hole blocking layer or the electron transport layer comprises one of the following compounds:

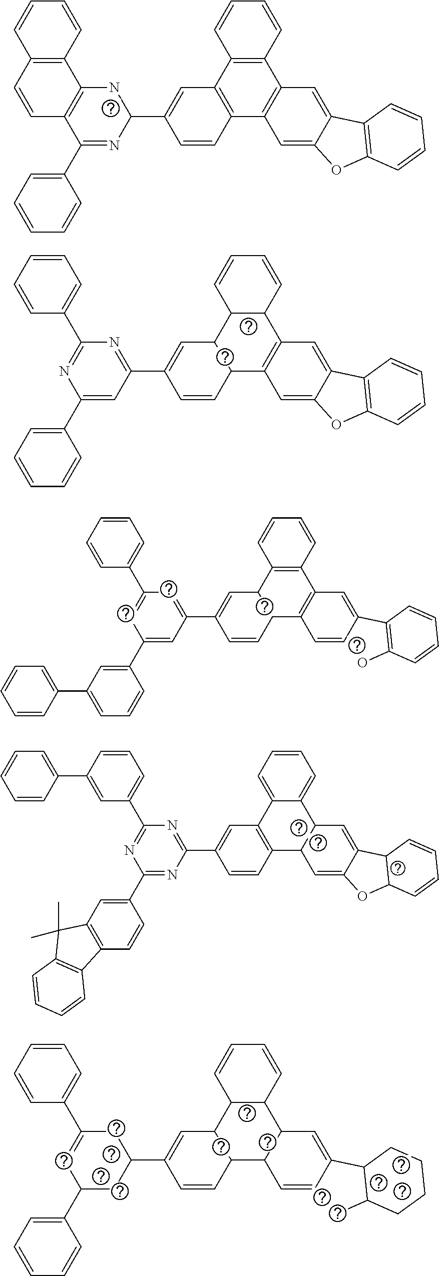

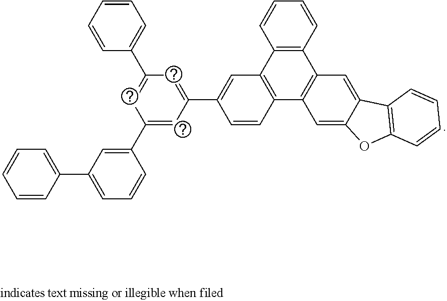 [SMILES: c1ccc(-c2cccc(-c3cc(-c4ccc5c(c4)c4ccccc4c4cc6c(cc54)oc4ccccc46)nc(-c4ccccc4)n3)c2)cc1]

17. The blue organic light emitting diode of claim 16, wherein the hole blocking layer or the electron transport layer comprises the same compound.
